(19) United States
(12) Patent Application Publication
(10) Pub. No.: US 2019/0270778 A1
(43) Pub. Date: Sep. 5, 2019

(54) Cyclic Peptides As C5a Receptor Antagonists

(71) Applicant: Pfizer Inc., New York, NY (US)

(72) Inventors: Ye Che, Niantic, CT (US); Yiqing Feng, Groton, CT (US); Matthew Merrill Hayward, Old Lyme, CT (US); David Hepworth, Concord, MA (US); Peter Jones, Sharon, MA (US); Kaila Neelu, Lexington, MA (US); Nikolaos Papaioannou, Newton, MA (US)

(73) Assignee: Pfizer Inc., New York, NY (US)

(21) Appl. No.: 16/319,985

(22) PCT Filed: Jul. 17, 2017
(86) PCT No.: PCT/IB2017/054314

§ 371 Date: Jan. 23, 2019

Related U.S. Application Data
(60) Provisional application No. 62/368,262, filed on Jul. 29, 2016.
(60) Provisional application No. 62/517,215, filed on Jun. 9, 2017.

(51) Int. Cl. C07K 7/56; A61K 38/08; A61P 13/12

(52) U.S. Cl. CPC C07K 7/56; A61P 13/12; A61K 38/08

(57) ABSTRACT

The invention relates to cyclic peptide derivatives, to their use in medicine, to compositions containing them, to processes for their preparation and to intermediates used in such processes. More particularly the invention relates to cyclic peptide C5a receptor antagonists of formula (Ia) or formula (Ib), or pharmaceutically acceptable salts thereof, wherein R^{1a}, R^{1b}, R², R³ and R⁴ areas defined in the description. C5a receptor antagonists are potentially useful in the treatment of a wide range of 1 disorders, including inflammatory disorders and immune disorders.

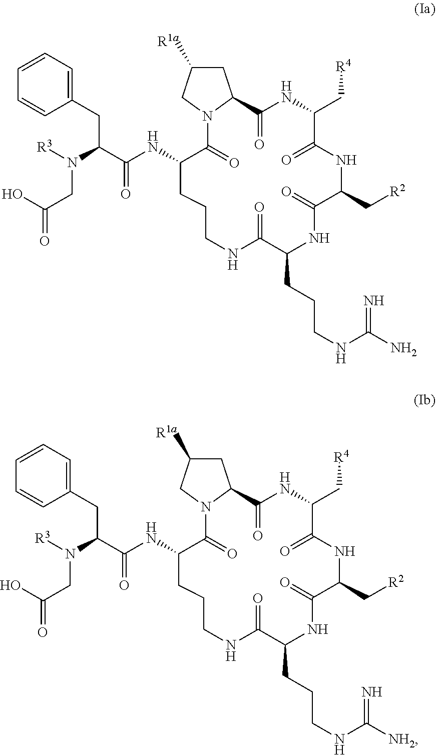

The invention relates to cyclic peptide derivatives, to their use in medicine, to compositions containing them, to processes for their preparation and to intermediates used in such processes.

The complement system is a part of the innate immune system that enhances (complements) the ability of antibodies and phagocytic cells to clear microbes and damaged cells from an organism. It consists of a group of proteins (complement components, C) that are normally present in blood in an inactive state. When stimulated by one of several triggers, the complement system initiates an enzyme cascade that helps defend against infection. However, uncontrolled activation or inadequate regulation of the complement system is related to several inflammatory and degenerative diseases; a review is provided by Morgan and Harris (Nature Reviews Drug Discovery 14, 857-877 (2015)).

There are three pathways of complement system activation: the classical, the lectin, and the alternative pathways. Microorganisms, antibodies or cellular components can activate these pathways resulting in the formation of protease complexes known as the C3-convertase and the C5-convertase. Each pathway converges into a final common pathway when C3-convertase cleaves C3 into fragments C3a and C3b. An overview of the complement system is provided by Sarma and Ward (Cell Tissue Res. 2011 January; 343(1): 227-235).

C5a is generated in the complement cascade by cleavage of C5 by C5-convertase enzyme. C5a is both an anaphylatoxin, causing increased expression of adhesion molecules on endothelium and contraction of smooth muscle, and a chemotactant, initiating accumulation of complement and phagocytic cells at sites of infection or recruitment of antigen-presenting cells to lymph nodes.

C5a interacts with the C5a receptor, also known as C5a receptor 1 (C5AR1) or CD88, a membrane bound G-protein coupled receptor (GPCR), and triggers a number of pro-inflammatory effects. C5a is a potent chemotactant for polymorphonuclear leukocytes, bringing neutrophils, basophils, eosinophils and monocytes to sites of inflammation and/or cellular injury, and indeed is one of the most potent chemotactic agents known for a wide variety of inflammatory cell types.

Amongst other actions C5a: “primes” (prepares) neutrophils for various antibacterial functions (e.g. phagocytosis); stimulates the release of inflammatory mediators (e.g. histamines, TNF-u, IL-I, IL-6, IL-8, prostaglandins, and leukotrienes) and the release of lysosomal enzymes and other cytotoxic components from granulocytes; and promotes the production of activated oxygen radicals and the contraction of smooth muscle.

It is believed that C5a release is directly or indirectly responsible for many diseases and syndromes. Examples are sepsis, reperfusion injury, rheumatoid arthritis and immune complex associated diseases in general. An overview over C5a related diseases is provided by Guo and Ward (Annu. Rev. Immunol. 2005. 23:821-52).

Acute kidney injury (AKI), defined as a loss of renal function over just a few days, is a common and severe clinical problem (Seminars in Nephrology, Vol 33, No 6, November 2013, pp 543-556). Estimates of its prevalence vary, but can range from 20-50% of Intensive Care Unit (ICU) patients, and can be associated with mortality of more than 50% (Critical Care Research and Practice, Vol 2013 (2013), Article ID 479730, 9 pages). AKI can be caused by underlying renal disease or it can be due to renal injury. Ischemia/reperfusion is a common cause of AKI in hospitalized patients and is a major factor in the development of AKI after transplantation, cardiac surgery, and sepsis.

Despite medical advances in supportive care, AKI is associated with high morbidity and mortality. Tissue inflammation is central to the pathogenesis of renal injury, even after non-immune insults such as ischemia/reperfusion and toxins, and activation of the complement system is a critical cause of AKI. Furthermore, complement system activation within the injured kidney triggers many downstream inflammatory events that exacerbate injury to the kidney. Complement system activation may also account for the systemic inflammatory events that contribute to remote organ injury and patient mortality.

Certain molecules that modulate the effects of the complement system, such as peptidic C5a modulators, are known. WO99/00406 discloses cyclic agonists and antagonists of C5a receptors. WO03/033528 discloses cyclic peptides as g-protein-coupled receptor antagonists. WO2005/010030 and WO2006/074964 disclose C5a receptors antagonists.

However, there is an ongoing need to provide new C5a receptor antagonists that are good drug candidates, in particular molecules that are suitable for intravenous administration in a hospital setting.

Preferred compounds have one or more of the following properties:
a physical form that is stable;
easy formulation for parenteral administration;
good thermodynamic aqueous solubility at a moderate pH suitable for intravenous infusion (e.g. a pH of from 3 to 9), such as >5 mg/ml, e.g. >25 mg/ml;
potent inhibition of a functional response to C5a, such as oxidative burst and CD11b up regulation, preferably with a Kb of <100 nM, e.g. <20 nM according to the assays described herein; and/or
a short pharmacokinetic half-life, such as <2 hours, e.g. <1 hour, such that C5a receptor blockade rapidly ceases on cessation of compound administration.

We have now found new cyclic peptide C5a receptors antagonists.

According to a first aspect of the invention there is provided a compound of formula (Ia) or formula (Ib)

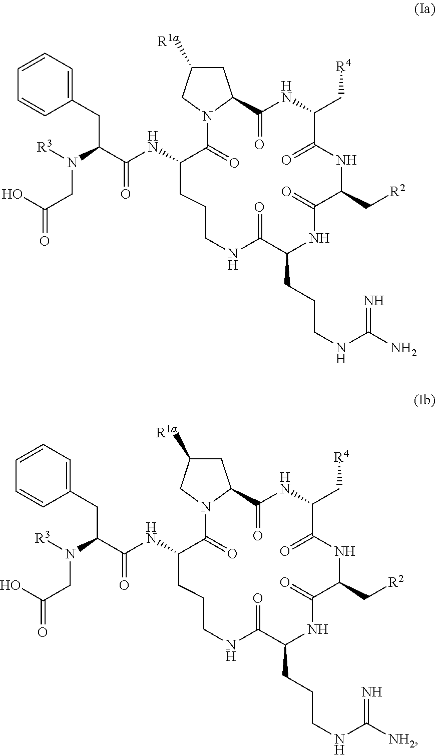

or a pharmaceutically acceptable salt thereof, wherein:
R^{1a} is H, OH, O(CH₂)—C(O)OR⁶, NH₂, NH—C(O)R⁵ or NH(CH₂)—C(O)OR⁶;
R^{1b} is NH₂, NH—C(O)R⁵ or NH(CH₂)—C(O)OR⁶;
R² is a 5-, 6-, 9- or 10-membered heteroaryl containing one, two or three nitrogen atoms and wherein the heteroaryl is optionally substituted on a ring carbon atom with one or two R⁷;
R³ is hydrogen or C₁-C₄ alkyl;
R⁴ is C₄-C₇ cycloalkyl substituted by [symbol] OH;
R⁵ is C₁-C₄ alkyl;
R⁶ is H or C₁-C₄ alkyl; and
R⁷ is C₁-C₄ alkyl or C₁-C₄ alkoxy.

Described below are a number of embodiments (E1) of this first aspect of the invention, where for convenience E1 is identical thereto.
E1 A compound of formula (Ia) or formula (Ib), or a pharmaceutically acceptable salt thereof, as defined above.
E2 A compound according to embodiment E1, or a pharmaceutically acceptable salt thereof, wherein R⁴ is cyclohexyl substituted by [symbol] OH.
E3 A compound according to either embodiment E1 or embodiment E2, or a pharmaceutically acceptable salt thereof, wherein R⁴ is

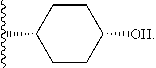 [SMILES: *[C@H]1CC[C@@H](O)CC1]

E4 A compound according to any one of embodiments E1 to E3, or a pharmaceutically acceptable salt thereof, wherein R^{1a} is OH, O(CH₂)—C(O)OR⁶, NH₂, NH—C(O)R⁵ or NH(CH₂)—C(O)OR⁶; or R^{1b} is NH₂, NH—C(O)R⁵ or NH(CH₂)—C(O)OR⁶.
E5 A compound according to any one of embodiments E1 to E4 of formula (Ia), or a pharmaceutically acceptable salt thereof, wherein R^{1a} is OH, O(CH₂)—C(O)OR⁶, NH₂, NH—C(O)R⁵ or NH(CH₂)—C(O)OR⁶.
E6 A compound according to embodiment E5 of formula (Ia), or a pharmaceutically acceptable salt thereof, wherein R^{1a} is OH or O(CH₂)—C(O)OH.
E7 A compound according to embodiment E6 of formula (Ia), or a pharmaceutically acceptable salt thereof, wherein R^{1a} is OH.
E8 A compound according to any one of embodiments E1 to E4 of formula (Ib), or a pharmaceutically acceptable salt thereof, wherein R^{1b} is NH₂, NH—C(O)R⁵ or NH(CH₂)—C(O)OR⁶.
E9 A compound according to embodiment E8, or a pharmaceutically acceptable salt thereof, wherein R^{1b} is NH₂, NH—C(O)CH₃ or NH(CH₂)—C(O)OH.
E10 A compound according to any one of embodiments E1 to E9, or a pharmaceutically acceptable salt thereof, wherein R² is a 9-membered heteroaryl containing one or two nitrogen atoms and wherein the heteroaryl is optionally substituted on a ring carbon atom with one or two R⁷.
E11 A compound according to any one of embodiments E1 to E10, or a pharmaceutically acceptable salt thereof, wherein R² is a C-linked heteroaryl selected from

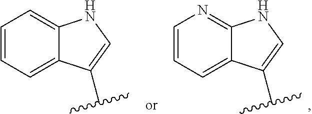 [SMILES: *c1c[nH]c2ccccc12 ; *c1c[nH]c2ncccc12]

wherein said heteroaryl is optionally substituted on a ring carbon atom with R⁷.
E12 A compound according to any one of embodiments E1 to E11, or a pharmaceutically acceptable salt thereof, wherein R² is the C-linked heteroaryl

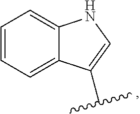 [SMILES: *c1c[nH]c2ccccc12]

wherein said heteroaryl is optionally substituted on a ring carbon atom with R⁷.
E13 A compound according to any one of embodiments E1 to E12, or a pharmaceutically acceptable salt thereof, wherein R² is the C-linked heteroaryl

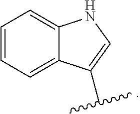 [SMILES: *c1c[nH]c2ccccc12]

E14 A compound according to any one of embodiments E1 to E12, or a pharmaceutically acceptable salt thereof, wherein R⁷ is methyl or methoxy.
E15 A compound according to any one of embodiments E1 to E14, or a pharmaceutically acceptable salt thereof, wherein R³ is H or methyl.
E16 A compound according to any one of embodiments E1 to E15, or a pharmaceutically acceptable salt thereof, wherein R³ is H.
E17 A compound according to embodiment E1, or a pharmaceutically acceptable salt thereof, selected from:
N-[(2S)-1-{[(3R,6S,9S,15S,19R,20aS)-9-(3-carbamimidamidopropyl)-19-hydroxy-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine (alternative name for Example 1);
N-[(2S)-1-{[(3R,6S,9S,15S,19R,20aS)-9-(3-carbamimidamidopropyl)-19-(carboxymethoxy)-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine (alternative name for Example 2);
N-[(2S)-1-({(3R,6S,9S,15S,19R,20aS)-9-(3-carbamimidamidopropyl)-19-hydroxy-3-[(cis-4-hydroxycyclohexyl)methyl]-6-[(4-methyl-1H-pyrazol-1-yl)methyl]-1,4,7,10,16-pentaoxoicosahydropyrrolo[1,2-a][1,4,7,10,13]pentaazacyclooctadecin-15-yl}amino)-1-oxo-3-phenylpropan-2-yl]glycine (alternative name for Example 3);
N-[(2S)-1-({(3R,6S,9S,15S,19R,20aS)-9-(3-carbamimidamidopropyl)-19-hydroxy-3-[(cis-4-hydroxycyclohexyl)methyl]-6-[(5-methoxy-1H-indol-3-yl)methyl]-1,4,7,10,16-pentaoxoicosahydropyrrolo[1,2-a][1,4,7,10,13]pentaazacyclooctadecin-15-yl}amino)-1-oxo-3-phenylpropan-2-yl]glycine (alternative name for Example 4);
N-[(2S)-1-{[(3R,6S,9S,15S,20aS)-9-(3-carbamimidamidopropyl)-3-[(cis-4-hydroxycyclohexyl)methyl]-1,4,7,10,16-pentaoxo-6-(1H-pyrrolo[2,3-b]pyridin-3-ylmethyl)icosahydropyrrolo[1,2-a][1,4,7,10,13]pentaazacyclooctadecin-15-yl]amino}-1-oxo-3-phenylpropan-2-yl]glycine (alternative name for Example 5);
N-[(2S)-1-{[(3R,6S,9S,15S,19R,20aS)-9-(3-carbamimidamidopropyl)-19-hydroxy-3-[(cis-4-hydroxycyclohexyl)methyl]-1,4,7,10,16-pentaoxo-6-(1H-pyrrolo[2,3-b]pyridin-3-ylmethyl)icosahydropyrrolo[1,2-a][1,4,7,10,13]pentaazacyclooctadecin-15-yl]amino}-1-oxo-3-phenylpropan-2-yl]glycine (alternative name for Example 6);
N-[(2S)-1-{[(3R,6S,9S,15S,19S,20aS)-19-amino-9-(3-carbamimidamidopropyl)-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine (alternative name for Example 7);
N-[(2S)-1-{[(3R,6S,9S,15S,19R,20aS)-19-amino-9-(3-carbamimidamidopropyl)-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine (alternative name for Example 8);
N-[(2S)-1-{[(3R,6S,9S,15S,19R,20aS)-19-(acetylamino)-9-(3-carbamimidamidopropyl)-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine (alternative name for Example 9);
N-[(2S)-1-{[(3R,6S,9S,15S,19S,20aS)-19-(acetylamino)-9-(3-carbamimidamidopropyl)-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine (alternative name for Example 10);
N-[(2S)-1-{[(3R,6S,9S,15S,19R,20aS)-9-(3-carbamimidamidopropyl)-19-hydroxy-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]-N-methylglycine (alternative name for Ex 11);
{[(2S)-1-{[(3R,6S,9S,15S,19S,20aS)-9-(3-carbamimidamidopropyl)-19-[(carboxymethyl)amino]-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]amino}acetic acid (alternative name for Example 12);
N-{(2S)-1-{[(3R,6S,9S,15S,19R,20aS)-9-(3-carbamimidamidopropyl)-19-hydroxy-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-(3,5-dideuterophenyl)propan-2-yl}glycine (alternative name for Example 13);
{[(2S)-1-{[(3R,6S,9S,15S,19R,20aS)-9-(3-carbamimidamidopropyl)-19-[(carboxymethyl)amino]-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]amino}acetic acid (alternative name for Example 14);
N-[(2S)-1-({(3R,6S,9S,15S,19R,20aS)-9-(3-carbamimidamidopropyl)-19-hydroxy-3-[(cis-4-hydroxycyclohexyl)methyl]-6-[(6-methoxy-1H-indol-3-yl)methyl]-1,4,7,10,16-pentaoxoicosahydropyrrolo[1,2-a][1,4,7,10,13]pentaazacyclooctadecin-15-yl}amino)-1-oxo-3-phenylpropan-2-yl]glycine (alternative name for Example 15);
((S)-1-(((3R,6S,9S,15S,19R,20aS)-6-((1H-indazol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine (Example 16); and
((S)-1-(((3R,6S,9S,15S,19R,20aS)-6-((6-ethyl-1H-indol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine (Example 17).

In compounds of formula (Ia) and formula (Ib):
Alkyl and alkoxy groups, containing the requisite number of carbon atoms, can be unbranched or branched. Examples of alkyl include methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy.
Examples of cycloalkyl include cyclobutyl, cyclopentyl, cyclohexyl and cycloheptyl.
Unless otherwise specified to the contrary, heteroaryl may be ‘C-linked’ or ‘N-linked’. The term ‘C-linked’ means that the heteroaryl is joined via a ring carbon. The term ‘N-linked’ means that the heteroaryl is joined via a ring nitrogen.
Specific examples of 5-, 6-, 9- and 10-membered heteroaryl include pyrrolyl, pyrazolyl, imidazoyl, triazolyl, pyridyl, pyridazinyl, pyrimidinyl, pyrazinyl, triazinyl, indolyl, isoindolyl, benzimidazolyl, indazolyl, benzotriazolyl, pyrrolo[2,3-b]pyridyl, pyrrolo[2,3-c]pyridyl, pyrrolo[3,2-c]pyridyl, pyrrolo[3,2-b]pyridyl, imidazo[4,5-b]pyridyl, imidazo[4,5-c]pyridyl, pyrazolo[4,3-d]pyridyl, pyrazolo[4,3-c]pyridyl, pyrazolo[3,4-c]pyridyl, pyrazolo[3,4-b]pyridyl, indolizinyl, imidazo[1,2-a]pyridyl, imidazo[1,5-a]pyridyl, pyrazolo[1,5-a]pyridyl, pyrrolo[1,2-b]pyridazinyl, quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-d]pyrazinyl and pyrido[3,4-b]pyrazinyl.

Unless the context requires a specific reference to formula (Ia), formula (Ib) or both formula (Ia) and formula (Ib), hereinafter formula (I) is used to refer collectively to both formulae (Ia) and (Ib),

Hereinafter, all references to compounds of the invention include compounds of formula (I) or pharmaceutically acceptable salts, solvates, or multi-component complexes thereof, or pharmaceutically acceptable solvates or multi-component complexes of pharmaceutically acceptable salts of compounds of formula (I), as discussed in more detail below.

Preferred compounds of the invention are compounds of formula (I) or pharmaceutically acceptable salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

The skilled person will appreciate that the aforementioned salts include ones wherein the counterion is optically active, for example d-lactate or 1-lysine, or racemic, for example dl-tartrate or dl-arginine.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of formula (I) or pharmaceutically acceptable salts thereof may exist in both unsolvated and solvated forms. The term ‘solvate’ is used herein to describe a molecular complex comprising a compound of formula (I) or a pharmaceutically acceptable salt thereof and one or more pharmaceutically acceptable solvent molecules, for example, ethanol. The term ‘hydrate’ is employed when said solvent is water. Pharmaceutically acceptable solvates in accordance with the invention include those wherein the solvent of crystallization may be isotopically substituted, e.g. D₂O, d₆-acetone and d₆-DMSO.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) of compounds of formula (I) or pharmaceutically acceptable salts thereof wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

The compounds of the invention may be administered as prodrugs. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in a compound of formula (I) with certain moieties known to those skilled in the art as ‘pro-moieties’ as described, for example, in “Design of Prodrugs” by H Bundgaard (Elsevier, 1985).

Examples of prodrugs include phosphate prodrugs, such as dihydrogen or dialkyl (e.g. di-tert-butyl) phosphate prodrugs. Further examples of replacement groups in accordance with the foregoing examples and examples of other prodrug types may be found in the aforementioned references.

Also included within the scope of the invention are metabolites of compounds of formula (I), that is, compounds formed in vivo upon administration of the drug. Some examples of metabolites in accordance with the invention include, where the compound of formula (I) contains a phenyl (Ph) moiety, a phenol derivative thereof (-Ph>-PhOH).

Formulae (Ia) and (Ib) contain asymmetric carbon atoms and are stereospecifically defined. The skilled person will appreciate that where R^{1a} and R^{1b} are, respectively, NH₂, NH—C(O)R⁵ or NH(CH₂)—C(O)OR⁶), formulae (Ia) and (Ib) define pairs of epimers. The invention includes all such epimers and mixtures thereof.

The skilled person will also appreciate that one or more substituents in formula (I) may introduce one or more additional asymmetric carbon atoms. Compounds of the invention containing said one or more additional asymmetric carbon atoms can exist as two or more stereoisomers; included within the scope of the invention are all such stereoisomers of the compounds of the invention and mixtures of two or more thereof.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Chiral chromatography using sub- and supercritical fluids may be employed. Methods for chiral chromatography useful in some embodiments of the present invention are known; see, for example, Smith, Roger M., Loughborough University, Loughborough, UK; Chromatographic Science Series (1998), 75 (Supercritical Fluid Chromatography with Packed Columns), pp. 223-249 and references cited therein.

Mixtures of stereoisomers may be separated by conventional techniques known to those skilled in the art; see, for example, “Stereochemistry of Organic Compounds” by E. L. Eliel and S. H. Wilen (Wiley, New York, 1994.

Where structural isomers are interconvertible via a low energy barrier, tautomeric isomerism (‘tautomerism’) and conformational isomerism can occur.

Tautomerism can take the form of proton tautomerism in compounds of formula (I) containing, for example, an amide group (i.e. amide-imidic acid tautomerism), or so-called valence tautomerism in compounds which contain an aromatic moiety. While, for conciseness, the compounds of formula (I) have been drawn herein in a single tautomeric form, all possible tautomeric forms are included within the scope of the invention.

Conformational isomerism is a form of stereoisomerism in which the isomers can be interconverted exclusively by rotations about single bonds. Such isomers are generally referred to as conformational isomers or conformers and, specifically, as rotamers. The amides of formula (I) can exist as rotamers. While, for conciseness, the compounds of formulae (I) have been drawn in a single conformational form, all possible conformers are included within the scope of the invention.

The scope of the invention includes all crystal forms of the compounds of the invention, including racemates and racemic mixtures (conglomerates) thereof. Stereoisomeric conglomerates may also be separated by the conventional techniques described herein just above.

The scope of the invention includes all pharmaceutically acceptable isotopically-labelled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention include isotopes of: hydrogen, such as ²H and ³H; carbon, such as ¹¹C, ¹³C and ¹⁴C; nitrogen, such as ¹³N and ¹⁵N; and oxygen, such as ¹⁵O, ¹⁷O and ¹⁸O.

Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium (D), i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁵O and ¹³N, can be useful in Positron Emission Topography (PET) studies for examining substrate receptor occupancy.

Of particular interest are compounds of formula (I) wherein one or more H are replaced by D. In one embodiment of the invention the phenyl ring in formula (I) is substituted with one or more D. In another embodiment, the invention provides 3,5-dideuterophenyl compounds of formula (Ia^{D}) or formula (Ib^{D}):

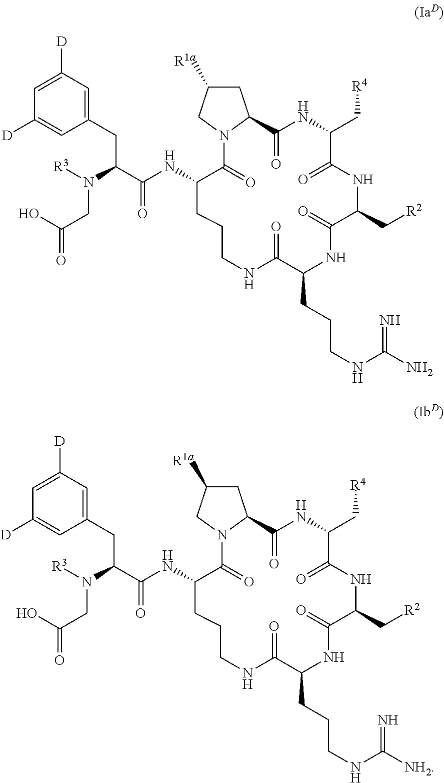

The skilled person will appreciate that embodiments E2 to E16 apply to the compounds of formula (Ia^{D}) and formula (Ib^{D}) just as they do to the compounds of formula (Ia) and formula (Ib). In relation to the compounds of formula (Ia^{D}) and formula (Ib^{D}), such embodiments are referred to as E2^{D} to E16^{D}.

The compound N-{(2S)-1-{[(3R,6S,9S,15S,19R,20aS)-9-(3-carbamimidamidopropyl)-19-hydroxy-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-(3,5-dideuterophenyl)propan-2-yl}glycine is therefore an embodiment of both formula (Ia) and formula (Ia^{D}).

Isotopically-labeled compounds of formula (I) can generally be prepared by conventional techniques known to those skilled in the art or by processes analogous to those described in the accompanying examples and preparations using an appropriate isotopically-labeled reagent in place of the non-labeled reagent previously employed.

Also within the scope of the invention are intermediate compounds as hereinafter defined, all salts, solvates and complexes thereof and all solvates and complexes of salts thereof as defined hereinbefore for compounds of formula (I). The invention includes all polymorphs of the aforementioned species and crystal habits thereof.

When preparing a compound of formula (I) in accordance with the invention, a person skilled in the art may routinely select the form of intermediate which provides the best combination of features for this purpose. Such features include the melting point, solubility, processability and yield of the intermediate form and the resulting ease with which the product may be purified on isolation.

The compounds of the invention may be prepared by any method known in the art for the preparation of compounds of analogous structure. In particular, the compounds of the invention can be prepared by the procedures described by reference to the schemes that follow, or by the specific methods described in the examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of compounds of formula (I). It will be further appreciated that it may be necessary or desirable to carry out the transformations in a different order from that described in the schemes, or to modify one or more of the transformations, to provide the desired compound of the invention.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect hydroxyl, carboxyl and/or amino groups. The protecting groups used in the preparation of the compounds of the invention may be used in conventional manner; see, for example, those described in ‘Greene's Protective Groups in Organic Synthesis’ by Theodora W Greene and Peter G M Wuts, fifth edition, (John Wiley and Sons, 2014), incorporated herein by reference, and in particular chapters 2, 5 and 7 respectively, which also describes methods for the removal of such groups.

In the following general processes R¹ to R⁴ are as previously defined for a compound of formula (I) unless otherwise stated. While the schemes describe the preparation of compounds of formula (Ia), the skilled person will appreciate that they are equally suitable for the provision of compounds of formula (Ib).

The compounds of formula (I) may be prepared according to either Scheme 1 or Scheme 2, depending on whether the side chain amino acid group containing R³ is installed at the beginning of the sequence prior to macrocyclisation, or at the end of the sequence following macrocyclisation. Both schemes make use of 2-chlorotrityl chloride (CTC) resin based solid phase synthesis (SPS) techniques with an initial loading step using a protected version of the amino acid ornithine.

According to a 1st process, compounds of formula (Ia) may be prepared by Scheme 1.

According to Scheme 1, N^{α}-fluorenylmethyloxycarbonyl-N^{δ}-allyloxycarbonyl-L-ornithine was loaded onto the resin followed by removal of the fluorenylmethyloxycarbonyl (FMOC) group providing a compound of formula (V), which allowed for subsequent installation of the desired side chain amino acid containing R³ selectively onto the free ^{α}amino group. Removal of the allyloxycarbonyl group then allowed for amino acid coupling on the free ^{δ}amino group. Subsequent peptide chain extension using well established SPS techniques using FMOC protected amino acids provided a resin-bound hexapeptide of formula (IVa) with one free amino group. Subsequent cleavage from the resin provided a hexapeptide of formula (IIIa) with one free amino group and one free carboxylic acid group that underwent macrolactamisation to provide the cyclic peptide framework of formula (IIa). Removal of the various acid labile protecting groups PG₁, PG₂ PG₃ and Pbf followed by purification provided the final cyclic peptides of formula (Ia).

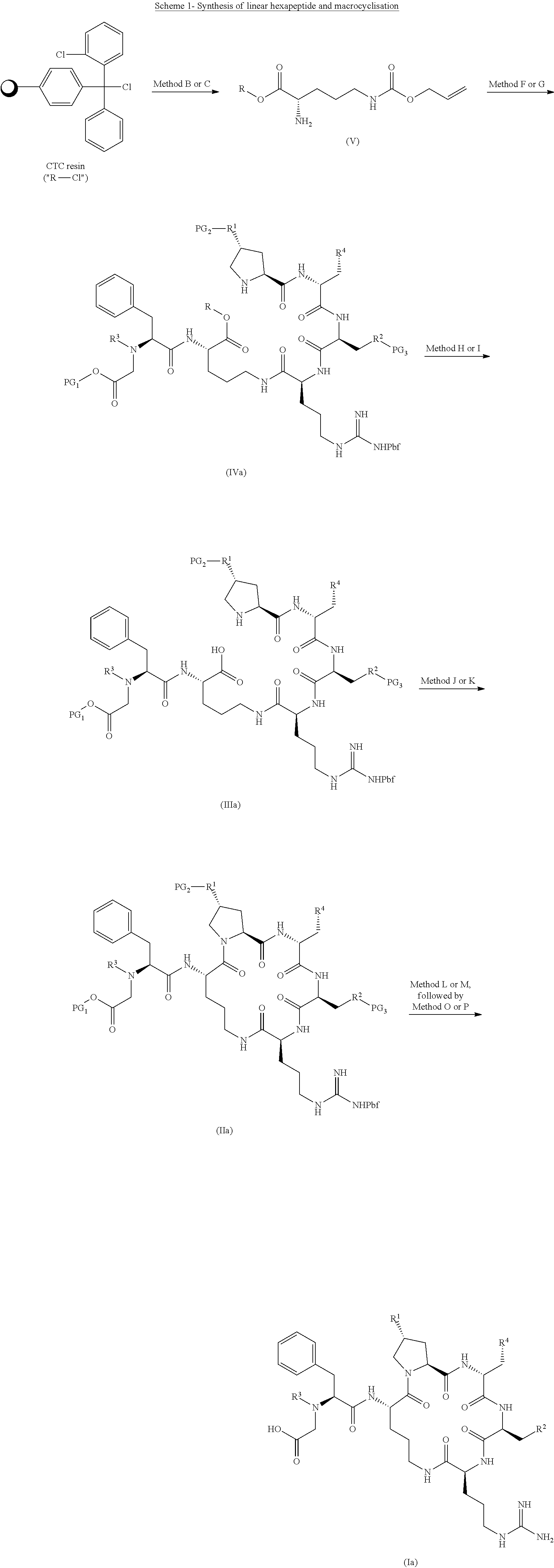

According to a 2^{nd} process, compounds of formula (Ia) may be prepared by Scheme 2.

According to scheme 2, N^{α}-tert-butyloxycarbonyl-N^{δ}-fluorenylmethyloxycarbonyl-L-ornithine was loaded onto the resin followed by removal of the FMOC group providing a compound of (X), which allowed for amino acid coupling on the free ^{δ}amino group. Subsequent peptide chain extension using well established SPS techniques using FMOC protected amino acids provided a resin-bound pentapeptide of formula (IXa) with one free amino group. Subsequent cleavage from the resin provided a pentapeptide of formula (VIIIa) with one free amino group and one free carboxylic acid group that underwent macrolactamisation to provide the cyclic peptide framework of formula (VIIa). Removal of various acid labile protecting groups, including the N^{α}-tert-butyloxycarbonyl group from the ornithine residue and PG₂ PG₃ and Pbf, provided cyclic pentapeptide of formula (VIa) with one free amino group. Installation of the side chain amino acid onto the free amino group, followed by purification, provided the final cyclic peptides of formula (Ia).

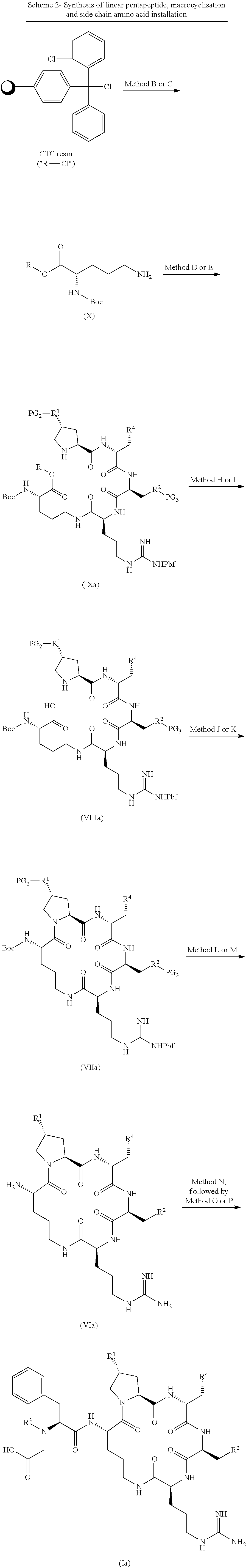

CTC resin and the necessary amino acids are commercially available, known from the literature, easily prepared by methods well known to those skilled in the art, or otherwise can be made according to preparations described herein.

All new processes for preparing compounds of formula (I), and corresponding new intermediates employed in such processes, form further aspects of the present invention.

Compounds of the invention intended for pharmaceutical use may be administered as crystalline or amorphous products or may exist in a continuum of solid states ranging from fully amorphous to fully crystalline. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

In another aspect the invention provides a pharmaceutical composition comprising a compound of the invention and a pharmaceutically acceptable excipient.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

The compounds of the invention may be administered parenterally, i.e. directly into the blood stream, into muscle, or into an internal organ.

Intravenous administration, in particular, represents a convenient means for administering the compounds of the invention. Other suitable means for parenteral administration include intraarterial, intraperitoneal, intrathecal, intraventricular, intraurethral, intrasternal, intracranial, intramuscular and subcutaneous.

Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Conveniently compounds of the invention are formulated for immediate release

Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

Other modes of administration include oral, topical, inhaled/intranasal, rectal/intravaginal and ocular/aural administration. Formulations suitable for these modes of administration include immediate and/or modified release. Modified release formulations include delayed-, sustained-, pulsed-, controlled-, targeted and programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, including hydroxypropyl beta cyclodextrin and sodium sulphobutylether beta cyclodextrin, examples of which may be found in International Patent Applications Nos. WO 91/11172, WO 94/02518 and WO 98/55148.

For administration to human patients, the total daily dose of the compounds of the invention is typically in the range 1 mg to 10 g, such as 60 mg to 6 g, for example 100 mg to 1 g depending, of course, on the mode of administration and efficacy. For example, intravenous administration may require a total daily dose of from 400 mg to 800 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

As noted above, the compounds of the invention are useful because they exhibit pharmacological activity in animals, i.e. C5a receptor antagonism. More particularly, the compounds of the invention are of use in the treatment of disorders for which a C5a receptor antagonist is indicated. Preferably the animal is a mammal, more preferably a human.

In a further aspect of the invention there is provided a compound of the invention for use as a medicament.

In a further aspect of the invention there is provided a compound of the invention for use in the treatment of a disorder for which a C5a receptor antagonist is indicated.

In a further aspect of the invention there is provided use of a compound of the invention for the preparation of a medicament for the treatment of a disorder for which a C5a receptor antagonist is indicated.

In a further aspect of the invention there is provided a method of treating a disorder in an animal (preferably a mammal, more preferably a human) for which a C5a receptor antagonist is indicated, comprising administering to said animal a therapeutically effective amount of a compound of the invention.

Disorders for which a C5a receptor antagonist is indicated include inflammatory disorders and immune disorders.

Inflammatory disorders include, but are not limited to: sepsis, such as sepsis associated with acute kidney, lung, liver, heart and brain injury; anaphylaxis; transplant rejection, such as that associated with the kidney, lung, heart, liver and pancreas; systemic vasculitis, such as anti-neutrophil cytoplasmic antibody associated vasculitis; ocular diseases, such as macular degeneration and uveitis; pulmonary diseases, such as asthma and chronic obtrusive pulmonary disease (COPD); acute exacerbation of an inflammatory disorder, such as COPD or systemic lupus erythematosus (SLE); and ischemia reperfusion injury of the kidney, lung, liver, heart and brain.

Immune disorders include, but are not limited to: hemolytic uremic syndrome (HUS), including atypical HUS (aHUS); rheumatoid arthritis; Gullain-Barré syndrome; Crohn's disease; ulcerative colitis; myasthenia gravis; anti-phospholipid syndrome; pemphigus; pemphigoid; SLE; IgA nephropathy; and lupus nephritis.

A disorder of particular interest is acute kidney injury (AKI), including AKI caused by:
an underlying renal disease, such as glomerulonephritis or hemolytic uremic syndrome; or by
drugs, ischemia, infections, or nephrotoxic metabolites.

A C5a receptor antagonist may be usefully combined with another pharmacologically active compound, or with two or more other pharmacologically active compounds. Such combinations offer the possibility of significant advantages, including patient compliance, ease of dosing and synergistic activity.

In such combinations the compound of the invention may be administered simultaneously, sequentially or separately in combination with the other therapeutic agent or agents.

The one or more additional therapeutic agents may be selected from any of the agents or types of agent that follow:
1) a TNF-alpha inhibitor, such as adalimumab, certolizumab pegol, etanercept, infliximab or golimumab;
2) an alkaline phosphatase, such as a recombinant alkaline phosphatase (e.g. PF-06853082);
3) a toll-like receptor (TLR) antagonist, such as a neutralizing antibody;
4) another agent for treating inflammatory disease and/or autoimmune disease, such as methotrexate, leflunomide, sulfasalazine or azathioprine;
5) an antihistamine receptor antagonist, such as an H₁ receptor antagonist (e.g. diphenhydramine);
6) a formyl peptide receptor (FPR) antagonist, such as an FPR-1 receptor antagonist;
7) another complement system inhibitor, such as a Factor B inhibitor, a C5a neutralizing antibody or a C5L2 inhibitor;
8) a chemokine or chemokine receptor (CCR) neutralizing antibody or antagonist, such as a CCR2 receptor antagonist (e.g. PF-04136309) or a CCR2/5 dual receptor antagonist, such as PF-04634817;
9) a kinase inhibitor, including:
   a. an inhibitor of a Janus Kinase, such JAK 1 (e.g. PF-04965842), JAK2 or JAK3 (e.g. tofacitinib or PF-06651600);
   b. an inhibitor of a tyrosine kinase, such as TYK2;
   c. an inhibitor of p38;
   d. an inhibitor of MAPK;
   e. an inhibitor of Syk;
   f. an inhibitor of IKK2; or
   g. an inhibitor of one or more of the above kinases, such as a TYK2/JAK1 inhibitor (e.g. PF-06700841);
   h. an inhibitor of interleukin-1 receptor-associated kinase (IRAK), such as an inhibitor of IRAK4 (e.g. PF-06650833);
   i. an inhibitor of Bruton's tyrosine kinase (BTK);
10) an interleukin (IL) inhibitor or IL receptor inhibitor, such as an IL-1 inhibitor (e.g. anakinra), an IL-6 inhibitor (e.g. tocilizumab), an IL-12/IL-23 inhibitor (e.g. ustekimumab), or an IL-33 inhibitor (e.g. PF-0817024);
11) an integrin inhibitor, such as natalizumab;
12) a non-steroidal antiinflammatory drug (NSAID), such as a propionic acid derivative (e.g. alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen).

It is within the scope of the invention that two or more pharmaceutical compositions, at least one of which contains a compound of the invention, may conveniently be combined in the form of a kit suitable for coadministration of the compositions. Thus the kit of the invention comprises two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

In another aspect the invention provides a pharmaceutical product (such as in the form of a kit) comprising a compound of the invention together with one or more additional therapeutically active agents as a combined preparation for simultaneous, separate or sequential use in the treatment of a disorder for which a C5a receptor antagonist is indicated.

It is to be appreciated that all references herein to treatment include curative, palliative and prophylactic treatment.

In the non-limiting Examples and Preparations that are set out later in the description, and in the aforementioned Schemes, the following the abbreviations, definitions and analytical procedures may be referred to:

AcOH is acetic acid;
APCI is atmospheric pressure chemical ionization:
aq is aqueous;
boc is tert-butyloxycarbonyl;
(boc)₂O is di-tert-butyl dicarbonate;
° C. is degrees celcius;
Cbz-Cl is carboxybenzyl chloride (also known as benzyl chloroformate);
CDCl₃ is deuterochloroform;
CD₃OD is deuteromethanol;
CTC is 2-chlorotrityl;
DBU is 1,8-diazabicyclo[5.4.0]undec-7-ene;
DCE is dichloroethane;
DCM is dichloromethane (also known as methylene chloride);
DEA is diethylamine;
DIPEA is diisopropylethyl amine;
DMAP is dimethylaminopyridine;
DME is dimethoxyethane;

### DMF is N,N-Dimethylformamide;

DMSO is dimethylsulphoxide;
d₆-DMSO is deuterodimethylsulphoxide;
ESCI is electrospray chemical ionization;
ESI is electrospray ionization;
EtOAc is ethyl acetate;
Fmoc (FMOC) is fluorenylmethyloxycarbonyl;
Fmoc-OSu is N-(9-fluorenylmethoxycarbonyloxy)succinimide;
g is gram;
HCl is hydrochloric acid;
HATU is 1-[bis(dimethylamino)methylene]-1H-1,2,3-triazolo[4,5-b]pyridinium 3-oxide hexafluorophosphate;
HBTU is O-benzotriazole-N,N,N′,N′-tetramethyl-uronium-hexafluoro-phosphate);
HFIPA is hexafluoroisopropanol;
HPLC is high pressure liquid chromatography;
i-PrOH is isopropanol;
L is litre;
LC-MS is liquid chromatography mass spectrometry;
M is molar;
MeCN is acetonitrile;
MeOH is methanol;
meq is molar equivalent;
mg is milligram;
MHz is mega Hertz;
min is minutes;
mL is millilitre;
mmol is millimole;
mol is mole;
MTBE is methyl tertiary-butyl ether;
MS m/z is mass spectrum peak;
NaHCO₃ is sodium hydrogencarbonate;
NaOH is sodium hydroxide;
NH₃ or NH₄OH is ammonia or ammonium hydroxide;

### NMM N-methylmorpholine;

Pbf is (2,2,4,6,7-pentamethyl-2,3-dihydrobenzofuran-5-yl)sulfonyl;
PE is petroleum ether;
pH is power of hydrogen;
r.t. is room temperature;
SFC is supercritical fluid chromatography;
SPPS is solid phase peptide synthesis;
TBAI is tetrabutylammonium iodide;
TBME is tert-butyl dimethyl ether;
TEA is triethylamine;
TFA is trifluoroacetic acid;
THF is tetrahydrofuran;
t_{R} is retention time;
μL is microlitre; and
μmol is micromole.

The following procedures were used in relation to the Examples that follow.

### Method A: LC-MS Method for Monitoring Progress of Solid Phase Reactions

A small amount of CTC resin-bound peptide (ca. 2 to 5 mg) was treated with 20% HFIPA in DCM at r.t. for 5 min. The volatiles were evaporated under a stream of nitrogen, and the residue was dissolved in methanol, filtered and analyzed by Waters LC-MS.

### LC-MS Condition:

Column: Waters Acquity HSS T3, 2.1 mm×50 mm, C18, 1.7 μm; Temperature: 60° C.; mobile phase A: 0.1% formic acid in water (v/v); mobile phase B: 0.1% formic acid in acetonitrile (v/v); flow 1.25 ml/min.
1.5 min Run: Initial conditions: A-95%:B-5%; hold at initial from 0.0-0.1 min; Linear Ramp to A-5%:B-95% over 0.1-1.0 m in; hold at A-5%:B-95% from 1.0-1.1 min; return to initial conditions 1.1-1.5 min.
3 min Run: Initial conditions: A 95%:B 5%; hold at initial conditions from 0.0 to 0.1 min, then linear ramp to A 5%:B 95% from 0.1 to 2.6 min; hold at A 5%:B 95% from 2.6 to 2.95 min, then return to initial conditions from 2.95 to 3.0 min.
Detectors: Waters Acquity PDA; 200-450 nm scan; 1.2 nm interval
   Waters Acquity ELS detector; drift tube 65° C.
   Waters SQ MS (single quad) Tune: ESI-3.5 kV Capillary/APCI (in ESCI mode)-0.3 μA Corona Pin, 30 V Cone, Source 150° C., Desolvation 475° C., Desolvation Gas N2 400 L/hr
MS Methods: ESCI (ESI+/−, APCI+/−), 100-2000 m/z scan, 0.4 sec scan time, Centroid

Injection Volume: 5 μl

Method B: Loading of Fmoc-Protected Amino Acid onto CTC Resin (Less than 1 mmol Scale)

In a SPPS tube, CTC resin (CAS 42074-68-0, commercially available from ChemImpex, catalogue number 04250, 1.0-1.7 meq/g, 1.0 equiv.) was mixed with a solution of a selected Fmoc-protected amino acid (1.1 equiv.) and DIPEA (6 equiv.) in a mixed solvent of DMF/DCM (1:10 v/v, 12 ml/mmol of CTC resin). The mixture was shaken at r.t. for 5 h. Anhydrous methanol (16 equiv.) was then added to cap any unreacted CTC resin. After being shaken at r.t. for another 30 min, the resin was filtered out, washed with DMF (3×10 ml), DCM (3×10 ml), MeOH (3×10 ml), and DMF (3×10 ml). For Fmoc removal the resin was then treated with 20% v/v piperidine in DMF (10 ml) at r.t. on a shaking bed for 30 min. The resin was then filtered, washed with DMF (3×10 ml), DCM (3×10 ml), MeOH (3×10 ml) and dried completely under vacuum to afford the CTC resin-bound amino acid, which was used in solid phase synthesis directly without any further purification. The resin loading rate was estimated based on the weight increase compared to the non-loaded CTC resin.

Method C: Loading of Fmoc-Protected Amino Acid onto 2-Chlorotrityl (CTC) Resin (Between 1.0 and 100 mmol Scale)

In an SPPS vessel equipped with an overhead mechanical stirrer, CTC resin (1.0-1.7 meq/g, 1.0 equiv.) was mixed with a solution of a selected Fmoc-protected amino acid (1.1 equiv.) and DIPEA (6 equiv.) in a mixed solvent of DMF/DCM (1:10 v/v, 6.6 ml/mmol of CTC resin). The mixture was gently stirred at r.t. for 5 h. Anhydrous methanol (16 equiv.) was added to cap any unreacted CTC resin. After being stirred at r.t. for another 30 min, the solution was removed from the resin by vacuum filtration. The resin-bound product was washed with DMF (3×200 ml), DCM (3×200 ml), MeOH (3×200 ml), and DMF (3×200 ml). For Fmoc removal the resin was then treated with 20% v/v piperidine in DMF (300 ml) at r.t. with gentle stirring for 30 min. The resin was then filtered under vacuum, washed with DMF (3×200 ml), DCM (3×200 ml), MeOH (3×100 ml) and dried completely under vacuum to afford the CTC resin-bound amino acid which was used in solid phase synthesis directly without any further purification. The resin loading rate was estimated based on the weight increase compared to the non-loaded resin.

Method D: Linear Pentapeptide Precursors, SPS (Less than 1 mmol Scale)

To a SPPS tube containing a CTC resin-bound amino acid (1.0 equiv.) was added a Fmoc-protected amino acid (1.5 equiv.), HBTU (1.5 equiv.), DMF (12 ml) and DIPEA (3 equiv.). The SPPS tube was capped and shaken at r.t. on a shaking bed for 2 h or until LC-MS indicated completion of the reaction using Method A. The reaction solution was then removed from the SPPS tube by vacuum filtration to afford the resin-bound product, which was rinsed with DMF (3×10 ml), DCM (3×10 ml), MeOH (3×10 ml) and DMF (3×10 ml). For removal of the Fmoc group, the resin was subsequently treated with 10 ml of 20% v/v piperidine in DMF at r.t. and placed on a shaking bed for 30 min, or until LC-MS indicated completion of the reaction using Method A. The resin was vacuum filtered and rinsed with DMF (3×12 ml), DCM (3×12 ml) and MeOH (3×10 ml), dried under vacuum to afford the resin-bound peptide with a free terminal amino group, which was used directly in the next amino acid coupling reaction without any further purification.

The above coupling/de-Fmoc procedure was repeated three more times, each time using a different amino acid respectively to afford the CTC resin-bound linear pentapeptide sequence with a free terminal amino group.

Method E: Linear Pentapeptide Precursors, SPS (Between 1.0 and 100 mmol Scale)

To a 500 ml SPPS vessel equipped with sintered filtering disc and overhead stirrer was added a CTC resin-bound amino acid (1.0 equiv, using Loading Method C), a Fmoc-protected amino acid (1.5 equiv.), HBTU (1.0 equiv.), DMF (100 ml) and DIPEA (3.0 equiv.). The mixture was gently stirred at r.t. for 2 h or until LC-MS indicated completion of the reaction using Method A. The reaction solution was then removed from the SPPS vessel by vacuum filtration, and the resin product was rinsed with DMF (3×100 ml), DCM (3×100 ml), MeOH (3×100 ml) and DMF (3×100 ml). For removal of the Fmoc group, the resin was subsequently treated with 100 ml of 20% v/v piperidine in DMF at r.t. and gently stirred for 30 min or until LC-MS indicated completion of the reaction using Method A. The resin was vacuum filtered and rinsed with DMF (3×120 ml), DCM (3×120 ml) and MeOH (3×100 ml), dried under vacuum to afford the resin-bound peptide with a free terminal amino group, which was used directly in the next amino acid coupling reaction without any further purification.

The above coupling/de-Fmoc procedure was repeated three more times, each time using a different amino acid respectively to afford the CTC resin-bound linear pentapeptide sequence with a free terminal amino group.

Method F: Linear Hexapeptide Precursors, SPS (Less than 1 mmol Scale)

### Side Chain Amino Acid Installation

N^{α}-Fmoc-N^{δ}-allyloxycarbonyl-L-ornithine (1.0 equiv) was loaded onto CTC resin using Method B. In a SPPS tube the resin-bound N^{α}-allyloxycarbonyl-L-ornithine was mixed with an N-protected amino acid (1.2 equiv.), HBTU (1.2 equiv.) and DMF (12 ml/mmol), and shaken at r.t. on a shaking bed for about 1 h or until LC-MS indicated the completion of the reaction using Method A. The resin was filtered, washed with DMF (3×10 ml), DCM (3×10 ml) and MeOH (3×10 ml), then dried under vacuum.

### Alloxycarbonyl Removal

The resin-bound dipeptide (1.0 equiv) was transferred to a round bottom flask equipped with magnetic stirrer. Under an atmosphere of nitrogen, DCM (8-9 ml/mmol), phenyl silane (16 equiv.) and tetrakis(triphenylphosphine)palladium (0.12 equiv.) were added sequentially. The resulting mixture was gently stirred (about 50 rpm) at r.t. for 1 h under N₂, then the resin-bound product was filtered, washed with DCM (3×10 ml), DMF (3×10 ml) and MeOH (3×10 ml), then dried under vacuum to afford the resin-bound dipeptide with a free ornithine δ-amino group.

### Further Coupling of Amino Acids

The resin-bound dipeptide with a free ornithine δ-amino group was transferred into a SPPS tube, then a Fmoc-protected amino acid (1.5 equiv.), HBTU (1.5 equiv.), DMF (12 ml) and DIPEA (3 equiv.) were added. The SPPS tube was capped and shaken at r.t. on a shaking bed for 2 h or until LC-MS indicated completion of the reaction using Method A. The reaction solution was then removed from the SPPS tube by vacuum filtration to afford the resin-bound product, which was rinsed with DMF (3×10 ml), DCM (3×10 ml), MeOH (3×10 ml) and DMF (3×10 ml). For FMOC removal, the resin was subsequently treated with 10 ml of 20% v/v piperidine in DMF at r.t. and placed on a shaking bed for 30 min or until LC-MS indicated completion of the reaction using Method A. The resin was vacuum filtered and rinsed with DMF (3×12 ml), DCM (3×12 ml) and MeOH (3×10 ml), then dried under vacuum to afford the resin-bound peptide with a free terminal amino group, which was used directly in the next amino acid coupling reaction without any further purification.

The above coupling/de-Fmoc procedure was repeated three more times, each time using a different amino acid respectively to afford the CTC resin-bound linear hexapeptide sequence with a free terminal amino group.

Method G: Linear Hexapeptide Precursor, SPS (Between 1.0 and 100 mmol Scale)

### Side Chain Amino Acid Installation

N^{α}-Fmoc-N^{δ}-allyloxycarbonyl-L-ornithine (1.0 equiv.) was loaded onto CTC resin using Method C. In a SPPS vessel equipped with overhead mechanical stirrer, the resin-bound N^{δ}-allyloxycarbonyl-L-ornithine was mixed with a N-protected amino acid (1.3 equiv), HBTU (1.3 equiv.) and DMF (6-6.6 ml/mmol of loaded resin). The mixture was gently stirred at r.t. for about 2 h or until LC-MS indicated the completion of the reaction using Method A. The resin was filtered, washed with DMF (3×200 ml), DCM (3×200 ml) and MeOH (3×100 ml), then dried under vacuum.

### Alloxycarbonyl Removal

To the SPPS vessel containing the resin-bound dipeptide (1.0 equiv) was added DCM (8-9 ml/mmol), phenyl silane (16 equiv.) and tetrakis(triphenylphosphine)palladium (0.12 equiv.) sequentially. The resulting mixture was gently stirred (about 50 rpm) at r.t. for 1 h under N₂. The resin-bound product was filtered, washed with DCM (3×200 ml), DMF (3×200 ml) and MeOH (3×100 ml) and then dried under vacuum to afford the resin-bound dipeptide with a free ornithine δ-amino group.

### Further Coupling of Amino Acids

To the SPPS vessel containing the resin-bound dipeptide with a free ornithine δ-amino group was added a Fmoc-protected amino acid (1.5 equiv.), HBTU (1.5 equiv.), DMF (6-7 ml/mmol of loaded resin) and DIPEA (3 equiv.). The mixture was stirred gently at r.t. for 2 h or until LC-MS indicated completion of the reaction using Method A. The reaction solution was then removed from the SPPS vessel by vacuum filtration to afford the resin-bound product, which was rinsed with DMF (3×200 ml), DCM (3×200 ml), MeOH (3×100 ml) and DMF (3×100 ml). For FMOC removal, the resin was subsequently treated with 20% v/v piperidine in DMF (8-9 ml/mmol of loaded resin) at r.t. with gentle stirring for 30 min or until LC-MS indicated completion of the reaction using Method A. The resin was vacuum filtered and rinsed with DMF (3×200 ml), DCM (3×200 ml) and MeOH (3×100 ml), then dried under vacuum to afford the resin-bound peptide with a free terminal amino group, which was used directly in the next amino acid coupling reaction without any further purification.

The above coupling/de-Fmoc procedure was repeated three more times, each time using a different amino acid respectively to afford the CTC resin-bound linear hexapeptide sequence with a free terminal amino group.

Method H: Cleavage of Linear Peptide from CTC Resin (Less than 1 mmol Scale)

The resin-bound pentapeptide or hexapeptide was treated with a solution of HFIPA in

DCM (20% v/v, 12 ml/mmol of loaded resin) in an SPPS tube with shaking on a shaking bed at r.t. for 30 min. The resin was filtered off and the filtrate collected. Volatiles were evaporated under vacuum to afford the linear pentapeptide or hexapeptide.

Method I: Cleavage of Linear Peptide from CTC Resin (Between 1.0 and 100 mmol Scale)

The resin-bound pentapeptide or hexapeptide was treated with a solution of HFIPA in DCM (20% v/v, 12 ml/mmol of substrate) in an SPPS vessel with gentle stirring at r.t. for 30 min. The mixture was filtered and filtrate collected. The resin was treated with another identical volume of 20% v/v HFIPA in DCM at r.t. upon gentle stirring for another 30 min, and filtered again. The filtrates were combined and evaporated under vacuum to dryness to afford the linear pentapeptide or hexapeptide.

Method J: Macrolactamization of Linear Pentapeptide or Hexapeptide Precursors (Less than 1 mmol Scale)

In a 250 ml round bottom flask, linear pentapeptide or hexapeptide solid (1.0 equiv.) was dissolved in a mixed solvent of DMF and THF (1:10 v/v) to afford a solution of 0.013 M concentration. Upon stirring, HATU (1.05 equiv.) and N-methyl morpholine (3 equiv.) were added. The resulting mixture was stirred at r.t. for 15-60 min or until LC-MS indicated completion of the reaction (Method A). Solvents were evaporated and the residue diluted with diethyl ether and ethyl acetate (1:1 v/v), washed with 0.5 M HCl, saturated NaHCO₃, water and brine. The organic layer was separated, dried over Na₂SO₄, filtered and evaporated to dryness to afford the cyclic fully protected pentapeptide or hexapeptide.

Method K: Macrolactamization of Linear Pentapeptide or Hexapeptide Precursors (Between 1.0 and 100 mmol Scale)

In a 3 L round bottom flask, linear pentapeptide or hexapeptide solid (1.0 equiv.) was dissolved in a mixed solvent of DMF and THF (1:10 v/v) to afford a solution of 0.013 M concentration. This solution was then added dropwise into a solution of HATU (1.05 equiv.) and N-methyl morpholine (3 equiv.) in DMF (6 ml/mmol of HATU). The resulting mixture was stirred at r.t. for 15-60 min or until LC-MS indicated completion of the reaction (Method A). Solvents were removed by evaporation and the residue was diluted with diethyl ether and ethyl acetate (1:1 v/v, 15 ml/mmol of substrate), washed with 0.5 M HCl, saturated NaHCO₃, water and brine. The organic layer was separated, dried over Na₂SO₄, filtered and evaporated to dryness to afford the cyclic fully protected pentapeptide or hexapeptide.

Method L: Global Deprotection (Less than 1 mmol Scale)

Fully protected cyclic peptide (1.0 equiv.) was dissolved in a solution of HCl in HFIPA (1 M, made by addition of 12 M HCl into HFIPA, 30 equiv.) at 0° C. with stirring. The resulting solution was stirred at r.t. for 1 h or until LC-MS indicated completion of reaction (Method A). Volatiles were removed by evaporation and the residue was co-distilled 5 times with MeCN to remove excess HCl. The residue was then dissolved in water, extracted with MTBE and neutralized to pH 6-7 by addition of 6 M NaOH. The mixture was allowed to stir at r.t. for 12 h or until LC-MS (Method A) indicated that no indole-N-carbamic acid remained. The residue was then co-evaporated with MeCN under reduced pressure to remove water. MeOH was added and the solution left to stand at r.t. for about 2 h. Precipitated NaCl solid was filtered off. The filtrate was then evaporated to dryness to afford the crude fully deprotected cyclic peptide.

Method M: Global Deprotection (Between 1.0 and 100 mmol Scale)

To a solution of fully protected cyclic peptide (1.0 equiv.) in HFIPA (28 ml/mmol of cyclic peptide) at 0° C. was added a solution of concentrated HCl (12 M, 30 equiv.) with stirring. The resulting solution was stirred at r.t. for 1 h or until LC-MS indicated completion of reaction (Method A). Volatiles were removed by evaporation, and the residue was co-distilled 5 times with MeCN to remove excess HCl. The residue was then dissolved in water, extracted by MTBE to remove any non-polar impurities, and neutralized to pH 6-7 by addition of 6 M NaOH. The mixture was allowed to stir at r.t. for 12 h or until LC-MS (Method A) indicated that no indole-N-carbamic acid remained. The residue was then co-evaporated with MeCN under reduced pressure to remove water. MeOH was added and the solution left to stand at r.t. for about 2 h. Precipitated NaCl solid was filtered off. The filtrate was then evaporated to dryness to afford the crude fully deprotected cyclic peptide.

### Method N: Side Chain Amino Acid Installation on the Fully Deprotected Cyclic Pentapeptide and Final Deprotection

The fully deprotected cyclic pentapeptide (1.0 equiv.) was treated with a N-protected amino acid (1.1 equiv), HATU (1.1 equiv.) and 4-methylmorpholine (8 equiv.) in DMF (37 ml/mmol of cyclic peptide) and the reaction stirred at r.t. for 80 min or until LC-MS (Method A) indicated completion of the reaction. The volatiles were removed under vacuum. The residue was diluted with ethyl acetate, and then washed with water and brine. The organic layer was separated, dried over Na₂SO₄, filtered and evaporated to dryness to give a solid residue. This solid was treated with 1 M HCl in HFIPA at r.t. for 1 h resulting in removal of any additional tert-butyl ester and/or Boc groups to afford the crude final deprotected cyclic peptide.

### Method O: HPLC Purification Method 1

Column: Luna 200×25 mm (C18, 10 μm, 100 A)+Gemini 150×30 mm (C18, 5 μm, 110 A)

Mobile phase: A acetonitrile; B water with 0.1% TFA. Isocratic 22% A followed by gradient from 46 min to 50 min to 24% A.

Run Time: 60 min followed by column wash (95% A)

Detector: 214/254 nm. Flow: 20 ml/min

Load: 1 g of crude material dissolved in 20 ml of 10% acetonitrile in water.

Whole fractions containing pure cyclic peptides were combined and lyophilized directly to provide cyclic peptides as solid TFA salts. TFA salts were passed again through the same column using a mobile phase containing aqueous ammonium bicarbonate to provide the free zwitterionic form.

Mobile phase: A acetonitrile; B water with 0.01 M NH₄HCO₃. Isocratic 28% A followed by gradient to 33% A from 30 min to 35 min.

Run Time: 60 min followed by column wash (95% A)

Detector: 220/254 nm. Flow: 20 ml/min

Load: ˜1.1 g of crude dissolved in 7 ml of 5% acetonitrile in water

Whole fractions containing pure cyclic peptides were combined and lyophilized directly to provide compounds of formula (I).

### Method P: HPLC Purification Method 2

Column: 50×250 mm, Sunfire, C18, 5 μm.

Mobile phase: A acetonitrile; B water with 0.1% TFA. Isocratic 15% A followed by gradient to 18% A from 45 min to 60 min.

Run Time: 65 min followed by column wash (95% A)

Detector: 220 nm. Flow: 100 ml/min

Load: 300 mg crude dissolved in 3 ml of 20% acetonitrile in water.

Whole fractions containing pure cyclic peptide were combined and the acetonitrile removed by rotary evaporation. The solid peptide TFA salt remaining was dissolved in water and loaded onto a capture column. Following washing, elution using a mobile phase containing aqueous ammonium bicarbonate provided the compounds of formula (I).

### Column: PoraPak Reverse Phase.

Material loaded onto the capture column was washed by pure water, several bed column volumes until pH was close to neutral. The column was then washed with ammonium bicarbonate solution (1 g/L of water), until the pH of the washing solution was approximately 8. Whole fractions containing pure cyclic peptide were combined and the acetonitrile was removed by rotary evaporation. The resulting aqueous solution of cyclic peptide was lyophilized to provide the compounds of formula (I).

In the following non-limiting examples and preparations, ¹H nuclear magnetic resonance (NMR) spectra were in all cases consistent with the proposed structures. Characteristic chemical shifts (δ) are given in parts-per-million (ppm) downfield from tetramethylsilane using conventional abbreviations for designation of major peaks (such as s=singlet, br s=broad singlet, d=doublet, dd=double doublet; td=triple doublet, t is triplet, q is quartet and m is multiplet).

With reference to the following examples, the skilled person will appreciate that different chemical naming conventions may provide alternative names for the same compound.

### EXAMPLE 1

### ((S)-1-(((3R,6S,9S,15S,19R,20aS)-6-((1H-indol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

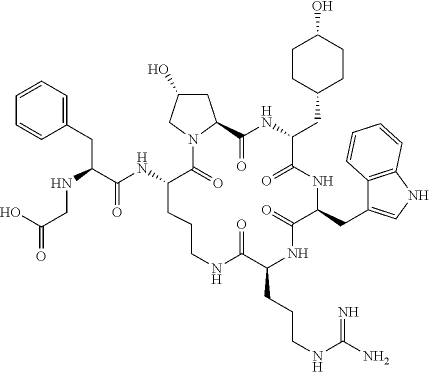 [SMILES: N=C(N)NCCC[C@@H]1NC(=O)[C@H](Cc2c[nH]c3ccccc23)NC(=O)[C@@H](C[C@H]2CC[C@@H](O)CC2)NC(=O)[C@@H]2C[C@@H](O)CN2C(=O)[C@@H](NC(=O)[C@H](Cc2ccccc2)NCC(=O)O)CCCNC1=O]

An alternative chemical name for Example 1 is: N-[(2S)-1-{[(3R,6S,9S,15S,19R,20aS)-9-(3-carbamimidamidopropyl)-19-hydroxy-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine.

### Loading.

Following Method C, N^{α}-Fmoc-N^{δ}-allyloxycarbonyl-L-ornithine (33.6 g, 76 mmol, 1.5 equiv.), CTC resin (1.7 meq, 30.0 g, 51.1 mmol, 1.0 equiv.), DIPEA (72 ml, 406 mmol, 8 equiv.) and a mixed solvent of DMF and DCM (1:10 v/v, 330 ml) were used. The mixture was gently stirred at r.t. for 5 h and capped by addition of MeOH (33 ml). Loading rate was estimated to be 81% or 41.3 mmol, based on weight increase of the resin. The resin was then treated with piperidine in DMF (20% v/v) to remove the Fmoc group to afford the CTC resin-bound N^{δ}-allyloxycarbonyl-L-ornithine.

### Side Chain Amino Acid Installation.

CTC resin-bound N^{δ}-allyloxycarbonyl-L-ornithine (46.6 g, 41.3 mmol), N-(2-(tert-butoxy)-2-oxoethyl)-N-(tert-butoxycarbonyl)-L-phenylalanine (20.4 g, 53.7 mmol, 1.30 equiv.), HBTU (21.0 g, 53.7 mmol, 1.30 equiv.), DIPEA (22.6 ml, 128 mmol, 3.1 equiv.) and DMF (250 ml) were gently stirred at r.t. for 120 min when LC-MS (Method A) indicated completion of the reaction. LC-MS (Method A, 3 min run): t_{R} 1.92 min, ESI− [M−H] calculated for C₂₉H₄₁N₃O₉ 576.6; found 576.5.

### Alloxycarbonyl Removal.

The resin-bound dipeptide (S)-5-(((allyloxy)carbonyl)amino)-2-((S)-2-((2-(tert-butoxy)-2-oxoethyl)(tert-butoxycarbonyl)amino)-3-phenylpropanamido)pentanoic acid (47.7 g, 34.2 mmol, 1.0 equiv.) was suspended in DCM (285 ml). The mixture was stirred with phenylsilane (70 ml, 547 mmol, 16 equiv.) and tetrakis(triphenylphosphine)palladium (4.74 g, 4.10 mmol, 0.12 equiv.) at r.t. for 1 h when LC-MS (Method A) indicated completion of the reaction. The resin was filtered out, washed, dried to afford resin-bound (S)-5-amino-2-((S)-2-((2-(tert-butoxy)-2-oxoethyl)(tert-butoxycarbonyl)amino)-3-phenylpropanamido)pentanoic acid. LC-MS (Method A, 3 min run): t_{R} 1.22 min, ESI-[M−H] calculated for C₂₅H₃₇N₃O₇ 491.6; found 492.4.

Further Coupling of Amino Acids and Cleavage of Linear Peptide from the Resin.

Preparation of (3R,6S,9S,15S)-15-((S)-2-((2-(tert-butoxy)-2-oxoethyl)(tert-butoxycarbonyl)amino)-3-phenylpropanamido)-1-((2S,4R)-4-(tert-butoxy)pyrrolidin-2-yl)-6-((1-(tert-butoxycarbonyl)-1H-indol-3-yl)methyl)-3-(((1s,4S)-4-hydroxycyclohexyl)methyl)-1,4,7,10-tetraoxo-9-(3-(3-((2,2,4,6,7-pentamethyl-2,3-dihydrobenzofuran-5-yl)sulfonyl)guanidino)propyl)-2,5,8,11-tetraazahexadecan-16-oic acid (the linear hexapeptide).

Resin-bound (S)-5-amino-2-((S)-2-((2-(tert-butoxy)-2-oxoethyl)(tert-butoxycarbonyl)amino)-3-phenylpropanamido)pentanoic acid (28.5 mmol, 1.0 equiv.) was subject to the solid phase coupling and Fmoc removal procedures described in Method G sequentially with N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (33.3 g, 42 mmol, 1.5 equiv.), N^{α}-(((9H-fluoren-9-yl)methoxy)carbonyl)-1-(tert-butoxycarbonyl)-L-tryptophan (30.0 g, 57 mmol, 2.0 equiv.), N-(9-Fluorenylmethyloxycarbonyl)-3-(cis-4-hydroxycyclohexyl)-D-alanine (15.2 g, 37 mmol, 1.3 equiv.), and trans-3-t-butoxy-N-(9-fluorenylmethyloxycarbonyl)-L-proline (15.2 g, 37 mmol, 1.3 equiv.) to afford the corresponding linear hexapeptide on resin. This resin-bound peptide was subjected to cleavage conditions described in Method I to afford the linear hexapeptide as a white solid, 33.8 g, 78%. ¹H NMR (400 MHz, d6-DMSO) δ 8.49 (br s, 1H), 8.38 (br s, 1H), 8.21-7.94 (m, 3H), 7.91 (br s, 1H), 7.70 (d, J=7.8 Hz, 1H), 7.51 (s, 1H), 7.42-7.16 (m, 5H), 5.17 (td, J=6.7, 13.6 Hz, 2H), 4.76-4.48 (m, 2H), 4.47-4.25 (m, 1H), 4.25-4.13 (m, 2H), 4.07 (br s, 1H), 3.93 (br s, 1H), 3.84-3.62 (m, 2H), 3.55 (dd, J=10.9, 18.7 Hz, 3H), 3.36 (br s, 4H), 3.16 (d, J=14.4 Hz, 3H), 3.12-2.87 (m, 6H), 2.77 (d, J=12.5 Hz, 1H), 2.45 (s, 2H), 2.02 (s, 2H), 1.97 (br s, 1H), 1.87-1.67 (m, 2H), 1.64 (s, 4H), 1.58 (br s, 1H), 1.48-1.35 (m, 13H), 1.34-1.24 (m, 9H), 1.23-1.09 (m, 7H), 1.06 (br s, 1H), 0.98 (d, J=16.0 Hz, 1H), 0.92-0.72 (m, 1H). LC-MS (Method A, 3 min run): t_{R} 1.96 min, ESI− [M−H] calculated for C₇₈H₁₁₆N₁₁O₁₈S 1525.9; found 1526.4.

### Macrolactamization.

Preparation of tert-butyl-3-(((3R,6S,9S,15S,19R,20aS)-19-(tert-butoxy)-15-((S)-2-((2-(tert-butoxy)-2-oxoethyl)(tert-butoxycarbonyl)amino)-3-phenylpropanamido)-3-(((1s,4S)-4-hydroxycyclohexyl)methyl)-1,4,7,10,16-pentaoxo-9-(3-(3-((2,2,4,6,7-pentamethyl-2,3-dihydrobenzofuran-5-yl)sulfonyl)guanidino)propyl)icosahydropyrrolo[1,2-a][1,4,7,10,13]pentaazacyclooctadecin-6-yl)methyl)-1H-indole-1-carboxylate (the cyclic peptide).

Following Method K for macrolactamization, linear (3R,6S,9S,15S)-15-((S)-2-((2-(tert-butoxy)-2-oxoethyl)(tert-butoxycarbonyl)amino)-3-phenylpropanamido)-1-((2S,4R)-4-(tert-butoxy)pyrrolidin-2-yl)-6-((1-(tert-butoxycarbonyl)-1H-indol-3-yl)methyl)-3-(((1s,4S)-4-hydroxycyclohexyl)methyl)-1,4,7,10-tetraoxo-9-(3-(3-((2,2,4,6,7-pentamethyl-2,3-dihydrobenzofuran-5-yl)sulfonyl)guanidino)propyl)-2,5,8,11-tetraazahexadecan-16-oic acid (33.8 g, 22.2 mmol, 1.0 equiv.) was reacted with HATU (9.36 g, 24.4 mmol, 1.1 equiv.) and 4-methylmorpholine (12.3 ml, 111 mmol, 5.0 equiv.) in THF (1400 ml) and DMF (175 ml) at r.t. for 30 min, at which point LC-MS (Method A) indicated completion of the reaction. The cyclic peptide was isolated as an off-white powder and was used without further purification, 29.1 g, 87%. LC-MS (Method A): t_{R} 2.44 min, ESI+[M+H] calculated for C₇₈H₁₁₄N₁₁O₁₇S 1509.9; found 1510.2.

### Global Deprotection.

### Preparation of ((S)-1-(((3R,6S,9S,15S,19R,20aS)-6-((1H-indol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine (the Title Compound of Example 1)

Following Method M, cyclic peptide tert-butyl 3-(((3R,6S,9S,15S,19R,20aS)-19-(tert-butoxy)-15-((S)-2-((2-(tert-butoxy)-2-oxoethyl)(tert-butoxycarbonyl)amino)-3-phenylpropanamido)-3-(((1s,4S)-4-hydroxycyclohexyl)methyl)-1,4,7,10,16-pentaoxo-9-(3-(3-((2,2,4,6,7-pentamethyl-2,3-dihydrobenzofuran-5-yl)sulfonyl)guanidino)propyl)icosahydropyrrolo[1,2a][1,4,7,10,13]pentaazacyclooctadecin-6-yl)methyl)-1H-indole-1-carboxylate (29.1 g, 19.3 mmol) was treated with 12 M HCl (48 ml) in HFIPA (529 ml) at r.t. for 90 min to afford a white powder, 21.1 g, which was subject to preparative HPLC purification using the method described in Method P to afford the title compound, Example 1, as a white solid, 7.4 g, 40.7%. ¹H NMR (400 MHz, d6-DMSO) δ 10.92 (br s, 1H), 9.08 (br s, 1H), 8.59 (br s, 1H), 8.34 (br s, 1H), 7.97 (br s, 1H), 7.95-7.81 (m, 2H), 7.69 (d, J=8.2 Hz, 2H), 7.52 (d, J=7.4 Hz, 1H), 7.36-7.15 (m, 8H), 7.14-6.95 (m, 3H), 6.90 (br s, 1H), 4.64 (d, J=6.6 Hz, 2H), 4.47 (br s, 1H), 4.24 (br s, 2H), 4.06 (br s, 1H), 3.93 (br s, 1H), 3.57 (br s, 1H), 3.45 (br s, 2H), 3.39-3.17 (m, 10H), 3.14-2.88 (m, 6H), 2.86-2.59 (m, 4H), 2.54-2.46 (m, 4H), 2.09 (s, 1H), 1.99 (br s, 1H), 1.86 (br s, 2H), 1.62 (br s, 1H), 1.56-1.40 (m, 3H), 1.36 (br s, 3H), 1.32-1.18 (m, 4H), 1.18-0.98 (m, 7H), 0.86 (br s, 3H). HRMS (m/z) [M+H]⁺ calculated for C₄₇H₆₆N₁₁O₁₀ 944.4989, found 944.4987.

Example 1 was also prepared by following the procedures described below:

### Loading.

Following Method C, N^{α}-t-Butyloxycarbonyl-N^{δ}-(9-fluorenylmethyloxycarbonyl)-L-ornithine (8.73 g, 19.2 mmol, 1.2 equiv.), DIPEA (18 ml, 13.4 g, 102 mmol, 6.5 equiv.), and CTC-resin (1.2 meq/g, 13.3 g, 16 mmol, 1.0 equiv.) were used in the loading step. Following FMOC removal the loading rate was estimated to be 3.6 mmol based on weight increase of the resin.

Further Coupling of Amino Acids and Cleavage of Linear Peptide from Resin.

Preparation of (3R,6S,9S,15S)-1-((2S,4R)-4-(tert-butoxy)pyrrolidin-2-yl)-6-((1-(tert-butoxycarbonyl)-1H-indol-3-yl)methyl)-15-((tert-butoxycarbonyl)amino)-3-(((1s,4S)-4-hydroxycyclohexyl)methyl)-1,4,7,10-tetraoxo-9-(3-(3-((2,2,4,6,7-pentamethyl-2,3-dihydrobenzofuran-5-yl)sulfonyl)guanidino)propyl)-2,5,8,11-tetraazahexadecan-16-oic acid (the linear pentapeptide).

Following Method E, N_{α}-(9-Fluorenylmethyloxycarbonyl)-N′,N″-bis-t-butyloxycarbonyl-L-arginine (3.50 g, 5.40 mmol, 1.5 equiv.), N^{α}-(((9H-fluoren-9-yl)methoxy)carbonyl)-1-(tert-butoxycarbonyl)-L-tryptophan (2.46 g, 4.68 mmol, 1.3 equiv.), N-(9-fluorenylmethyloxycarbonyl)-3-(cis-4-hydroxycyclohexyl)-D-alanine (2.15 g, 5.24 mmol, 1.3 equiv.) and trans-3-t-butoxy-N-(9-fluorenylmethyloxycarbonyl)-L-proline (2.19 g, 5.35 mmol, 1.3 equiv.) were used sequentially in each of the solid phase coupling and Fmoc removal procedures to afford the CTC resin-bound linear pentapeptide. Following Method I, the linear pentapeptide was cleaved from the resin to afford the title compound as a white solid, 5.59 g, quantitative yield (calculated based on 3.6 mmol loading). LC-MS (Method A, 3 min run): t_{R} 1.71 min, ESI− [M−H] calculated for C₆₃H₉₅N₁₀O₁₅S 1264.5, found 1264.1.

### Macrolactamization.

### Preparation of tert-butyl-3-(((3R,6S,9S,15S,19R,20aS)-19-(tert-butoxy)-15-((tert-butoxycarbonyl)amino)-3-(((1s,4S)-4-hydroxycyclohexyl)methyl)-1,4,7,10,16-pentaoxo-9-(3-(3-((2,2,4,6,7-pentamethyl-2,3-dihydrobenzofuran-5-yl)sulfonyl)guanidino)propyl)icosahydropyrrolo[1,2-a][1,4,7,10,13]pentaazacyclooctadecin-6-yl)methyl)-1H-indole-1-carboxylate (the Cyclic Peptide)

Following Method K, the linear pentapeptide (3R,6S,9S,15S)-1-((2S,4R)-4-(tert-butoxy)pyrrolidin-2-yl)-6-((1-(tert-butoxycarbonyl)-1H-indol-3-yl)methyl)-15-((tert-butoxycarbonyl)amino)-3-(((1s,4S)-4-hydroxycyclohexyl)methyl)-1,4,7,10-tetraoxo-9-(3-(3-((2,2,4,6,7-pentamethyl-2,3-dihydrobenzofuran-5-yl)sulfonyl)guanidino)propyl)-2,5,8,11-tetraazahexadecan-16-oic acid (5.48 g, 4.33 mmol) was dissolved in THF and DMF (10:1 v/v, 330 ml) and treated with HATU (1.91 g, 4.98 mmol, 1.15 equiv.) and 4-methylmorpholine (2.41 ml, 21.7 mmol, 5 equiv.) at r.t. for 15 min. The cyclic peptide was isolated as an off-white solid, 5.15 g, 95.3%. ¹H NMR (400 MHz, d6-DMSO) δ 8.55 (br s, 1H), 8.25 (br s, 1H), 8.02 (d, J=8.2 Hz, 1H), 7.78 (d, J=8.6 Hz, 1H), 7.58 (d, J=7.8 Hz, 1H), 7.54-7.49 (m, 1H), 7.36-7.28 (m, 1H), 7.27-7.20 (m, 1H), 6.90 (br s, 1H), 6.72 (d, J=7.0 Hz, 1H), 4.64 (d, J=5.5 Hz, 1H), 4.49-4.36 (m, 2H), 4.25 (br s, 1H), 4.18-4.08 (m, 2H), 3.67-3.57 (m, 1H), 3.11-2.89 (m, 6H), 2.43 (s, 3H), 2.01 (s, 4H), 1.88 (d, J=6.6 Hz, 1H), 1.82-1.73 (m, 1H), 1.62 (s, 10H), 1.52 (br s, 3H), 1.40 (s, 9H), 1.36 (br s, 13H), 1.21-1.13 (m, 3H), 1.00 (d, J=11.3 Hz, 2H). LC-MS (Method A, 3 min run): t_{R} 2.12 min, ESI+[M+H] calculated for C₆₃H₉₅N₁₀O₁₄S 1248.5; found 1248.9.

### Global Deprotection.

### Preparation of 1-(3-((3R,6S,9S,15S,19R,20aS)-6-((1H-indol-3-yl)methyl)-15-amino-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-9-yl)propyl)guanidine (the Deprotected Cyclic Peptide)

Following Method M, the cyclic peptide tert-butyl 3-(((3R,6S,9S,15S,19R,20aS)-19-(tert-butoxy)-15-((tert-butoxycarbonyl)amino)-3-(((1s,4S)-4-hydroxycyclohexyl)methyl)-1,4,7,10,16-pentaoxo-9-(3-(3-((2,2,4,6,7-pentamethyl-2,3-dihydrobenzofuran-5-yl)sulfonyl)guanidino)propyl)icosahydropyrrolo[1,2-a][1,4,7,10,13]pentaazacyclooctadecin-6-yl)methyl)-1H-indole-1-carboxylate (5.15 g, 4.11 mmol, 1.0 equiv.) was dissolved in HFIPA (110 ml) and treated with 12 M HCl (10 ml, 12 mmol, 30 equiv.) at r.t. for 1 h. Removal of excess HCl and NaCl provided the deprotected cyclic peptide which was used directly in the next step. LC-MS (Method A, 3 min run) t_{R} 0.59 min, ESI+[M+H] calculated for C₃₆H₅₅N₁₀O₇ 739.9; found 739.6.

### Side Chain Amino Acid Installation on the Fully Deprotected Cyclic Pentapeptide, and Final Deprotection

### Preparation of ((S)-1-(((3R,6S,9S,15S,19R,20aS)-6-((1H-indol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine (the Title Compound of Example 1)

Following Method N, cyclic pentapeptide 1-(3-((3R,6S,9S,15S,19R,20aS)-6-((1H-indol-3-yl)methyl)-15-amino-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-9-yl)propyl)guanidine (4.11 mmol, 1.0 equiv.), N-(2-(tert-butoxy)-2-oxoethyl)-N-(tert-butoxycarbonyl)-L-phenylalanine (1.72 g, 4.52 mmol, 1.10 equiv.), HATU (1.74 g, 4.52 mmol, 1.10 equiv.), 4-methylmorpholine (4 ml, 33 mmol, 8 equiv.) and DMF (150 ml) were stirred at r.t. for 80 min. The orange gummy crude residue isolated was treated with 1 M HCl in HFIPA (50 ml) at r.t. for 30 min when LC-MS (Method A) indicated completion of the reaction. The volatiles were removed under reduced pressure, and the residue dissolved in HFIPA (30 ml). This HFIPA solution was added slowly into MeCN (100 ml) to afford a pink suspension, which was evaporated to dryness, and further co-evaporated with EtOAc (50 ml×2) to afford a brown powder, 3.72 g. The powder was purified using Method P to afford the title compound, Example 1, as a white solid, 204 mg, recovery 21.5%. ¹H NMR (400 MHz, d6-DMSO) δ 10.92 (br s, 1H), 9.08 (br s, 1H), 8.59 (br s, 1H), 8.34 (br s, 1H), 7.97 (br s, 1H), 7.95-7.81 (m, 2H), 7.69 (d, J=8.2 Hz, 2H), 7.52 (d, J=7.4 Hz, 1H), 7.36-7.15 (m, 8H), 7.14-6.95 (m, 3H), 6.90 (br s, 1H), 4.64 (d, J=6.6 Hz, 2H), 4.47 (br s, 1H), 4.24 (br s, 2H), 4.06 (br s, 1H), 3.93 (br s, 1H), 3.57 (br s, 1H), 3.45 (br s, 2H), 3.39-3.17 (m, 10H), 3.14-2.88 (m, 6H), 2.86-2.59 (m, 4H), 2.54-2.46 (m, 4H), 2.09 (s, 1H), 1.99 (br s, 1H), 1.86 (br s, 2H), 1.62 (br s, 1H), 1.56-1.40 (m, 3H), 1.36 (br s, 3H), 1.32-1.18 (m, 4H), 1.18-0.98 (m, 7H), 0.86 (br s, 3H). HRMS (m/z) [M+H]⁺ calculated for C₄₇H₆₆N₁₁O₁₀ 944.4989, found 944.4987.

### EXAMPLE 2

### 2-(((3R,6S,9S,15S,19R,20aS)-6-((1H-indol-3-yl)methyl)-15-((S)-2-((carboxymethyl)amino)-3-phenylpropanamido)-9-(3-guanidinopropyl)-3-(((1s,4S)-4-hydroxycyclohexyl)methyl)-1,4,7,10,16-pentaoxoicosahydropyrrolo[1,2-a][1,4,7,10,13]pentaazacyclooctadecin-19-yl)oxy)acetic Acid

Following Method B, N^{α}-Fmoc-N^{δ}-allyloxycarbonyl-L-ornithine was loaded onto 2-chlorotrityl resin (CTC resin, 1.0 meq, 1.0 g, 1.0 mmol) and subsequently treated with piperidine in DMF (20% v/v) to afford the CTC resin-bound N^{δ}-allyloxycarbonyl-L-ornithine (1.0 mmol).

The hexapeptide linear sequence was subsequently assembled by following the coupling and Fmoc removal procedures described in Method F using sequentially N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (973 mg, 1.5 equiv.), N^{α}-(((9H-fluoren-9-yl)methoxy)carbonyl)-1-(tert-butoxycarbonyl)-L-tryptophan (790 mg, 1.5 equiv.), N-(9-fluorenylmethyloxycarbonyl)-3-(cis-4-hydroxycyclohexyl)-D-alanine (614 mg, 1.3 equiv.), and (2S,4R)-1-(((9H-fluoren-9-yl)methoxy)carbonyl)-4-(2-(tert-butoxy)-2-oxoethoxy)pyrrolidine-2-carboxylic acid (701 mg, 1.5 equiv.) to afford the linear hexapeptide on resin. This resin-bound product was subjected to cleavage conditions described in general procedure H to afford the linear hexapeptide as a white solid, 775 mg, 79%. LC-MS (Method A, 3 min run) t_{R} 1.97 min, ESI+[M+H] calculated for C₈₀H₁₁₈N₂O₂₀S 1585.9; found 1585.8.

The linear hexapeptide (775 mg, 0.489 mmol, 1.0 equiv.) was subject to macrolactamization conditions described in Method J to afford the fully protected cyclic peptide as a white solid, 638 mg, 83%. LC-MS (Method A, 3 min run) t_{R} 2.49 min, ESI+[M+H] calculated for C₈₀H₁₁₆N₁₁O₁₉S 1567.9; found 1567.9.

The fully protected cyclic peptide (638 mg, 0.407 mmol, 1.0 equiv.) was subject to the global deprotection conditions described in Method L, which afforded the fully deprotected cyclic peptide as a white powder, 435 mg. This material was purified by HPLC using Method O to afford the title compound, Example 2, as a white solid, 151 mg, 35.8%. ¹H NMR (400 MHz, d6-DMSO) δ 8.61 (d, J=6.5 Hz, 1H), 8.37 (br s, 1H), 7.89 (dd, J=7.8, 17.3 Hz, 1H), 7.49 (d, J=8.0 Hz, 1H), 7.38-7.13 (m, 2H), 7.11-6.94 (m, 1H), 6.46 (d, J=8.5 Hz, 1H), 5.01-4.90 (m, 1H), 4.74 (d, J=9.0 Hz, 1H), 4.54 (br s, 1H), 4.21 (br s, 1H), 4.01 (d, J=6.0 Hz, 1H), 3.80 (br s, 1H), 3.74-3.54 (m, 1H), 3.50 (br s, 1H), 3.26-3.08 (m, 1H), 3.06-2.86 (m, 1H), 2.79 (dd, J=7.5, 14.1 Hz, 1H), 2.70-2.57 (m, 1H), 2.07 (s, 1H), 1.87 (d, J=5.5 Hz, 1H), 1.68 (br s, 1H), 1.56 (d, J=17.1 Hz, 1H), 1.38 (br s, 1H), 1.27 (br s, 1H), 1.20 (br s, 1H), 1.16-0.92 (m, 1H), 0.87 (d, J=10.5 Hz, 1H), 0.71 (br s, 1H). LC-MS (Method A, 3 min run) t_{R} 0.76 min, ESI-[M−H] calculated for C₄₉H₆₇N₁₁O₁₂ 1001.1; found 1000.7.

### EXAMPLE 3

### ((S)-1-(((3R,6S,9S,15S,19R,20aS)-9-(3-guanidinopropyl)-19-hydroxy-3-(((1s,4S)-4-hydroxycyclohexyl)methyl)-6-((4-methyl-1H-pyrazol-1-yl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method B, N^{α}-Fmoc-N^{δ}-allyloxycarbonyl-L-ornithine was loaded onto 2-chlorotrityl resin (CTC resin, 1.0 meq, 1.0 g, 1.0 mmol) and subsequently treated with piperidine in DMF (20% v/v) to afford the CTC resin-bound N^{δ}-allyloxycarbonyl-L-ornithine (0.6 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (340 mg, 0.9 mmol, 1.5 equiv.), N^{α}-((9H-fluoren-9-yl)methoxy)carbonyl)-N^{ω}-((2,2,4,6,7-pentamethyl-2,3-dihydrobenzofuran-5-yl)sulfonyl)-L-arginine (585 mg, 0.9 mmol, 1.5 equiv.), (S)-2-((((9H-fluoren-9-yl)methoxy)carbonyl)amino)-3-(4-methyl-1H-pyrazol-1-yl)propanoic acid (350 mg, 0.9 mmol, 1.5 equiv.), N-(9-fluorenylmethyloxycarbonyl)-3-(cis-4-hydroxycyclohexyl)-D-alanine (360 mg, 0.9 mmol, 1.5 equiv.), and (2S,4R)-1-(((9H-fluoren-9-yl)methoxy)carbonyl)-4-(tert-butoxy)pyrrolidine-2-carboxylic acid (370 mg, 0.9 mmol, 1.5 equiv.) to afford the resin-bound linear hexapeptide. This resin-bound product was subject to cleavage conditions described in general procedure H to afford the linear hexapeptide as a white solid, 670 mg, 80%. LC-MS (Method A, 1.5 min run) t_{R} 0.97 min, ESI+[M/2+H] calculated for C₆₉H₁₀₈N₁₂O₁₆S 696.8; found 696.4.

The linear hexapeptide (670 mg, 0.48 mmol, 1.0 equiv.) was subject to the macrolactamization conditions described in Method J to afford the fully protected cyclic peptide as a white solid, 320 mg, 48%. LC-MS (Method A, 1.5 min run) t_{R} 0.98 min, ESI+[M/2+H] calculated for C₆₉H₁₀₆N₁₂O₁₅S 687.9; found 687.4.

The crude fully protected cyclic peptide (320 mg, 0.23 mmol) was subject to the global deprotection conditions described in Method L, followed by preparative HPLC purification using Method O to afford the title compound, Example 3, as white fluffy solid, 80 mg, 46%. ¹H NMR (400 MHz, d6-DMSO) δ 8.60 (br s, 1H), 8.45 (br s, 2H), 7.70 (d, J=8.5 Hz, 1H), 7.58 (t, J=5.6 Hz, 1H), 7.37-7.18 (m, 9H), 7.13 (br s, 3H), 4.64-4.53 (m, 2H), 4.50-4.38 (m, 3H), 4.38-4.23 (m, 2H), 4.23-4.02 (m, 3H), 3.88 (d, J=4.5 Hz, 2H), 3.68 (br s, 2H), 3.64-3.55 (m, 2H), 3.51 (d, J=14.1 Hz, 6H), 3.38 (br s, 59H), 3.07 (br s, 8H), 2.74 (br s, 2H), 2.50 (d, J=3.5 Hz, 27H), 2.01-1.90 (m, 6H), 1.79 (br s, 1H), 1.59 (d, J=3.0 Hz, 3H), 1.47 (br s, 4H), 1.43-1.28 (m, 9H), 1.25 (br s, 4H), 1.14 (br s, 2H). LC-MS (Method A, 1.5 min run) t_{R} 0.74 min, ESI+[M+H] calculated for C₄₃H₆₅N₁₂O₁₀ 909.5; found 909.4.

### EXAMPLE 4

### ((S)-1-(((3R,6S,9S,15S,19R,20aS)-9-(3-guanidinopropyl)-19-hydroxy-3-(((1s,4S)-4-hydroxycyclohexyl)methyl)-6-((5-methoxy-1H-indol-3-yl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method B, N^{α}-Fmoc-N^{δ}-allyloxycarbonyl-L-ornithine was loaded onto 2-chlorotrityl resin (CTC resin, 1.0 meq, 100 mg, 0.1 mmol) and subsequently treated with piperidine in DMF (20% v/v) to afford the CTC resin-bound N^{δ}-allyloxycarbonyl-L-ornithine (0.1 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (57 mg, 0.15 mmol, 1.5 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (98 mg, 0.15 mmol, 1.5 equiv.), (S)-2-((((9H-fluoren-9-yl)methoxy)carbonyl)amino)-3-(5-methoxy-1H-indol-3-yl)propanoic acid (69 mg, 0.15 mmol, 1.5 equiv.), N-(9-Fluorenylmethyloxycarbonyl)-3-(cis-4-hydroxycyclohexyl)-D-alanine (61 mg, 0.15 mmol, 1.5 equiv.), (2S,4R)-1-(((9H-fluoren-9-yl)methoxy)carbonyl)-4-(tert-butoxy)pyrrolidine-2-carboxylic acid (62 mg, 0.15 mmol, 1.5 equiv.) to afford the resin-bound linear hexapeptide. This resin-bound product was subject to cleavage conditions described in Method H to afford the linear hexapeptide as a yellow oil, 146 mg, crude yield 100%. LC-MS (Method A, 1.5 min run) t_{R} 1.00 min, ESI+[M/2+H] calculated for C₇₄H₁₁₁N₁₁O₁₇S 729.4; found 729.0.

The crude linear hexapeptide (146 mg, 0.10 mmol, 1.0 equiv.) was subject to the macrolactamization conditions described in Method J to afford the fully protected cyclic peptide as an off-white solid, which was used in the global deprotection step without further purification.

The fully protected cyclic peptide was subject to global deprotection condition described in Method L, followed by preparative HPLC purification using Method O to afford the title compound, Example 4, as white solid, 5.9 mg, 6.0%. ¹H NMR (400 MHz, d6-DMSO) δ 10.80-10.72 (m, 1H), 8.52 (d, J=5.5 Hz, 1H), 8.35 (br s, 1H), 8.27 (s, 1H), 8.02 (s, 1H), 7.86 (s, 1H), 7.37-7.12 (m, 13H), 7.04-6.91 (m, 2H), 6.76-6.67 (m, 1H), 4.41 (br s, 5H), 4.10 (br s, 19H), 3.93 (br s, 5H), 3.75 (s, 7H), 3.70 (s, 15H), 3.17 (s, 31H), 1.36 (br s, 3H), 1.31-1.17 (m, 5H), 1.15 (br s, 3H), 1.10 (br s, 4H). LC-MS (Method A, 1.5 min run) t_{R} 0.61 min, ESI+[M+H] calculated for C₄₈H₆₈N₁₁O₁₁S 975.1; found 974.6.

### EXAMPLE 5

### ((S)-1-(((3R,6S,9S,15S,20aS)-6-((1H-pyrrolo[2,3-b]pyridin-3-yl)methyl)-9-(3-guanidinopropyl)-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method B, N^{α}-Fmoc-N^{δ}-allyloxycarbonyl-L-ornithine was loaded onto 2-chlorotrityl resin (CTC resin, 1.0 meq, 200 mg, 0.2 mmol) and subsequently treated with piperidine in DMF (20% v/v) to afford the CTC resin-bound N^{δ}-allyloxycarbonyl-L-ornithine (0.2 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (114 mg, 0.3 mmol, 1.5 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (195 mg, 0.30 mmol, 1.5 equiv.), (S)-2-((((9H-fluoren-9-yl)methoxy)carbonyl)amino)-3-(1-(tert-butoxycarbonyl)-1H-pyrrolo[2,3-b]pyridin-3-yl)propanoic acid (159 mg, 0.30 mmol, 1.5 equiv.), N-(9-fluorenylmethyloxycarbonyl)-3-(cis-4-hydroxycyclohexyl)-D-alanine (1231 mg, 0.30 mmol, 1.5 equiv.) and (((9H-fluoren-9-yl)methoxy)carbonyl)-L-proline (101 mg, 0.30 mmol, 1.5 equiv.) to afford the resin-bound linear hexapeptide. This resin-bound product was subject to the cleavage conditions described in Method H to afford the linear hexapeptide as a yellow oil, 271 mg, crude yield 100%. LC-MS (Method A, 1.5 min run) t_{R} 0.95 min, ESI+[M/2+H-Boc] calculated for C₇₃H₁₀₆N₁₂O₁₆S 678.8; found 678.5.

The crude linear hexapeptide (271 mg, 0.20 mmol, 1.0 equiv.) was subject to the macrolactamization conditions described in Method J to afford the fully protected cyclic peptide as an off-white solid, 302 mg. This material was used in the global deprotection without further purification. LC-MS (Method A, 1.5 min run) t_{R} 0.95 min, ESI+[M/2+H] calculated for C₇₃H₁₀₆N₁₂O₁₆S 719.3; found 718.5.

The fully protected cyclic peptide (302 mg, 0.2 mmol) was subject to the global deprotection conditions described in Method L, followed by preparative HPLC purification using Method O to afford the title compound, Example 5, as a white solid, 12.3 mg, 6.6%. ¹H NMR (400 MHz, d6-DMSO) δ 11.44 (s, 1H), 8.53 (d, J=7.5 Hz, 1H), 8.29-8.20 (m, 3H), 8.05-7.84 (m, 4H), 7.30-7.15 (m, 9H), 7.14-6.95 (m, 4H), 4.55 (br s, 2H), 4.37-3.86 (m, 6H), 3.52 (br s, 7H), 3.35 (br s, 210H), 3.17 (s, 17H), 3.09 (d, J=17.1 Hz, 7H), 3.04-2.86 (m, 8H), 2.50 (d, J=3.5 Hz, 254H), 2.33 (br s, 10H), 2.07 (s, 3H), 1.85 (br s, 4H), 1.65 (br s, 3H), 1.52 (br s, 6H), 1.33 (br s, 6H), 1.28-1.17 (m, 4H), 1.07 (d, J=10.0 Hz, 7H). LC-MS (Method A, 1.5 min run) t_{R} 0.51 min, ESI+[M+H] calculated for C₄₆H₆₅N₁₂O₉ 927.1; found 929.6.

### EXAMPLE 6

### ((S)-1-(((3R,6S,9S,15S,19R,20aS)-6-((1H-pyrrolo[2,3-b]pyridin-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method B, N^{α}-Fmoc-N^{δ}-allyloxycarbonyl-L-ornithine was loaded onto 2-chlorotrityl resin (CTC resin, 1.0 meq, 200 mg, 0.2 mmol) and subsequently treated with piperidine in DMF (20% v/v) to afford the CTC resin-bound N^{δ}-allyloxycarbonyl-L-ornithine (0.2 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (114 mg, 0.3 mmol, 1.5 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (195 mg, 0.30 mmol, 1.5 equiv.), (S)-2-((((9H-fluoren-9-yl)methoxy)carbonyl)amino)-3-(1-(tert-butoxycarbonyl)-1H-pyrrolo[2,3-b]pyridin-3-yl)propanoic acid (159 mg, 0.30 mmol, 1.5 equiv.), N-(9-fluorenylmethyloxycarbonyl)-3-(cis-4-hydroxycyclohexyl)-D-alanine (123 mg, 0.30 mmol, 1.5 equiv.) and (2S,4R)-1-(((9H-fluoren-9-yl)methoxy)carbonyl)-4-(tert-butoxy)pyrrolidine-2-carboxylic acid (123 mg, 0.30 mmol, 1.5 equiv.) to afford the resin-bound linear hexapeptide. This resin-bound product was subject to the cleavage conditions described in Method H to afford the linear hexapeptide as a yellow oil, 286 mg, crude yield 100%. LC-MS (Method A, 1.5 min run) t_{R} 0.97 min, ESI+[M/2+H-Boc] calculated for C₇₂H₁₀₈N₁₂O₁₆S 714.8; found 714.4.

The crude linear hexapeptide (286 mg, 0.20 mmol, 1.0 equiv.) was subject to the macrolactamization conditions described in Method J to afford the fully protected cyclic peptide as an off-white solid, 282 mg. This material was used in the global deprotection without further purification. LC-MS (Method A, 1.5 min run) t_{R} 1.00 min, ESI+[M/2+H] calculated for C₇₇H₁₁₂N₁₂O₁₇S 755.9; found 755.5.

The fully protected cyclic peptide (282 mg, 0.2 mmol) was subject to the global deprotection conditions described in Method L, followed by preparative HPLC purification using Method O to afford the title compound, Example 6, as a white solid, 7.8 mg, 4.8%. ¹H NMR (400 MHz, d6-DMSO) δ 11.46 (br s, 1H), 8.54 (br s, 1H), 8.33 (br s, 1H), 8.28 (s, 1H), 8.21-8.13 (m, 1H), 8.10-8.00 (m, 1H), 7.93 (d, J=8.5 Hz, 1H), 7.34-7.17 (m, 6H), 7.02 (dd, J=4.3, 7.8 Hz, 2H), 4.59 (br s, 1H), 4.40 (br s, 1H), 4.24 (br s, 2H), 4.09 (br s, 7H), 3.89 (br s, 3H), 3.33 (br s, 189H), 3.17 (s, 23H), 3.07 (d, J=16.1 Hz, 4H), 3.02-2.85 (m, 4H), 2.50 (d, J=3.5 Hz, 24H), 2.33 (s, 5H), 2.01-1.80 (m, 3H), 1.49 (br s, 4H), 1.32 (br s, 3H), 1.08 (d, J=10.0 Hz, 4H), 0.82 (br s, 1H). LC-MS (Method A) t_{R} 0.57 min, ESI+[M+H] calculated for C₄₆H₆₅N₁₂O₁₀ 946.1; found 946.6.

### EXAMPLE 7

### ((S)-1-(((3R,6S,9S,15S,19S,20aS)-6-((1H-indol-3-yl)methyl)-19-amino-9-(3-guanidinopropyl)-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method C, N^{α}-Fmoc-N^{δ}-allyloxycarbonyl-L-ornithine was loaded onto 2-chlorotrityl resin (CTC resin, 1.0 meq, 3.0 g, 3.0 mmol) and subsequently treated with piperidine in DMF (20% v/v) to afford the CTC resin-bound N^{δ}-allyloxycarbonyl-L-ornithine (3.0 mmol).

The hexapeptide linear sequence was subsequently assembled by following the procedures described in Method G using sequentially N-(2-(tert-butoxy)-2-oxoethyl)-N-(tert-butoxycarbonyl)-L-phenylalanine (1.71 g, 4.5 mmol, 1.5 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (2.92 g, 4.5 mmol, 1.5 equiv.), N^{α}-(((9H-fluoren-9-yl)methoxy)carbonyl)-1-(tert-butoxycarbonyl)-L-tryptophan (1.92 g, 4.5 mmol, 1.5 equiv.), N-(9-fluorenylmethyloxycarbonyl)-3-(cis-4-hydroxycyclohexyl)-D-alanine (1.80 g, 4.5 mmol, 1.5 equiv.) and (2S,4S)-1-(((9H-fluoren-9-yl)methoxy)carbonyl)-4-((tert-butoxycarbonyl)amino)pyrrolidine-2-carboxylic acid (2.03 g, 4.5 mmol, 1.5 equiv.) to afford the resin-bound linear hexapeptide. This resin-bound product was subject to the cleavage conditions described in Method I to afford the linear hexapeptide as an oil, 4.41 g, 100%. LC-MS (Method A, 1.5 min run) t_{R} 0.90 min, ESI+[M/2+H] calculated for C₇₄H₁₀₈N₁₂O₁₇S 735.9; found 735.1.

The linear hexapeptide (4.41 g, 3 mmol, 1.0 equiv.) was subjected to the macrolactamization conditions described in Method K to afford the fully protected cyclic peptide as a yellow solid, 4.35 g, 100%. LC-MS (Method A, 1.5 min run) t_{R} 0.97 min, ESI+[M+H] calculated for C₈₀H₁₀₆N₁₁O₁₆S 1452.8; found 1452.2.

The fully protected cyclic peptide (4.35 g, 3 mmol, 1.0 equiv.) was subjected to the global deprotection condition described in Method M, followed by preparative HPLC purification using Method O to afford the title compound, Example 7, as a white solid, 312 mg, 10%. ¹H NMR (400 MHz, d6-DMSO) δ 10.90 (s, 1H), 8.50 (br s, 3H), 8.30 (s, 1H), 8.10 (d, J=7.5 Hz, 1H), 7.76 (t, J=5.5 Hz, 1H), 7.51 (d, J=8.0 Hz, 5H), 7.32 (d, J=8.5 Hz, 2H), 7.27-7.16 (m, 6H), 7.13 (s, J=4.4 Hz, 1H), 7.05 (t, J=7.5 Hz, 1H), 6.97 (t, J=6.7 Hz, 1H), 4.59 (d, J=6.5 Hz, 1H), 4.49 (br s, 1H), 4.25 (br s, 2H), 4.13 (d, J=7.0 Hz, 2H), 3.97 (br s, 2H), 3.77 (br s, 2H), 3.59 (br s, 3H), 3.43 (br s, 1H), 3.40-3.25 (m, 4H), 3.12-2.95 (m, 5H), 2.93-2.85 (m, 1H), 2.78 (d, J=6.0 Hz, 1H), 2.50 (d, J=1.5 Hz, 50H), 2.31 (d, J=14.1 Hz, 4H), 1.77 (br s, 2H), 1.61 (br s, 1H), 1.49 (br s, 3H), 1.40 (br s, 5H), 1.21 (br s, 6H), 1.14 (br s, 7H), 0.96 (br s, 2H). LC-MS (Method A, 1.5 min run) t_{R} 0.59 min, ESI+[M+H] calculated for C₄₇H₆₆N₁₂O₉944.1; found 944.6.

### EXAMPLE 8

### ((S)-1-(((3R,6S,9S,15S,19R,20aS)-6-((1H-indol-3-yl)methyl)-19-amino-9-(3-guanidinopropyl)-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method B, N^{α}-Fmoc-N^{δ}-allyloxycarbonyl-L-ornithine was loaded onto 2-chlorotrityl resin (CTC resin, 1.0 meq, 1.0 g, 1.0 mmol) and subsequently treated with piperidine in DMF (20% v/v) to afford the CTC resin-bound N^{δ}-allyloxycarbonyl-L-ornithine (0.6 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (342 mg, 0.9 mmol, 1.5 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (584 mg, 0.9 mmol, 1.5 equiv.), N^{α}-(((9H-fluoren-9-yl)methoxy)carbonyl)-1-(tert-butoxycarbonyl)-L-tryptophan (384 mg, 0.9 mmol, 1.5 equiv.), N-(9-Fluorenylmethyloxycarbonyl)-3-(cis-4-hydroxycyclohexyl)-D-alanine (360 mg, 0.9 mmol, 1.5 equiv.) and (2S,4R)-1-(((9H-fluoren-9-yl)methoxy)carbonyl)-4-((tert-butoxycarbonyl)amino)pyrrolidine-2-carboxylic acid (406 mg, 0.9 mmol, 1.5 equiv.) to afford the resin-bound linear hexapeptide. This resin-bound product was subject to cleavage conditions described in Method H to afford the linear hexapeptide as a crude oil, 774 mg, 100%. LC-MS (Method A, 1.5 min run) t_{R} 0.94 min, ESI+[M/2+H] calculated for C₇₄H₁₀₈N₁₂O₁₇S 735.9; found 735.6.

The linear hexapeptide (774 mg, 0.6 mmol, 1.0 equiv.) was subjected to the macrolactamization conditions described in Method J to afford the fully protected cyclic peptide as a yellow oil which was used in the next step without further purification. LC-MS (Method A, 1.5 min run) t_{R} 0.97 min, ESI+[M/2+Na] calculated for C₈₀H₁ₒ₆N₁₁O₁₆S 749.1; found 749.1.

The fully protected cyclic peptide was subjected to the global deprotection conditions described in Method L, followed by preparative HPLC purification using Method O to afford the title compound, Example 8, as a white solid, 37.6 mg, 6.6%. ¹H NMR (400 MHz, d6-DMSO) δ 10.91 (br s, 1H), 8.74 (br s, 1H), 8.63 (br s, 1H), 8.32 (s, 2H), 8.02 (br s, 1H), 7.88 (t, J=5.7 Hz, 1H), 7.63 (br s, 2H), 7.51 (d, J=8.0 Hz, 2H), 7.36-7.15 (m, 6H), 7.12-7.03 (m, 1H), 7.01-6.91 (m, 1H), 4.68 (br s, 1H), 4.57 (br s, 1H), 4.23 (br s, 3H), 4.07 (br s, 3H), 3.93 (br s, 4H), 3.80 (br s, 6H), 3.68 (br s, 7H), 3.56 (br s, 7H), 3.39 (br s, 3H), 3.33 (br s, 4H), 3.17 (s, 1H), 3.06 (d, J=15.6 Hz, 3H), 3.00-2.85 (m, 3H), 2.79 (br s, 1H), 2.68 (br s, 1H), 2.55-2.46 (m, 47H), 2.33 (br s, 2H), 2.02 (br s, 1H), 1.84 (br s, 2H), 1.62 (br s, 1H), 1.51 (br s, 3H), 1.34 (br s, 4H), 1.16 (d, J=12.5 Hz, 4H), 1.06 (br s, 4H), 0.83 (br s, 1H). LC-MS (Method A, 1.5 min run) t_{R} 0.97 min, ESI+[M/2+H] calculated for C₄₇H₆₆N₁₂O₉ 472.0; found 472.5.

### EXAMPLE 9

### ((S)-1-(((3R,6S,9S,15S,19R,20aS)-6-((1H-indol-3-yl)methyl)-19-acetamido-9-(3-guanidinopropyl)-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method B, N^{α}-Fmoc-N^{δ}-allyloxycarbonyl-L-ornithine was loaded onto 2-chlorotrityl resin (CTC resin, 1.0 meq, 1.0 g, 1.0 mmol) and subsequently treated with piperidine in DMF (20% v/v) to afford the CTC resin-bound N^{δ}-allyloxycarbonyl-L-ornithine (0.6 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (342 mg, 0.9 mmol, 1.5 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (584 mg, 0.9 mmol, 1.5 equiv.), N^{α}-(((9H-fluoren-9-yl)methoxy)carbonyl)-1-(tert-butoxycarbonyl)-L-tryptophan (384 mg, 0.9 mmol, 1.5 equiv.), N-(9-fluorenylmethyloxycarbonyl)-3-(cis-4-hydroxycyclohexyl)-D-alanine (360 mg, 0.9 mmol, 1.5 equiv.) and (2S,4R)-1-(((9H-fluoren-9-yl)methoxy)carbonyl)-4-(acetylamino)pyrrolidine-2-carboxylic acid (406 mg, 0.9 mmol, 1.5 equiv.) to afford the resin-bound linear hexapeptide. This resin-bound product was subject to the cleavage conditions described in Method H to afford the linear hexapeptide as a crude oil, 847 mg, 100%. LC-MS (Method A, 1.5 min run) t_{R} 0.86 min, ESI+[M+H] calculated for C₇₁H₁ₒ₃N₁₂O₁₆S 1412.7; found 1412.5.

The linear hexapeptide (847 mg, 0.6 mmol, 1.0 equiv.) was subjected to the macrolactamization conditions described in Method J to afford the fully protected cyclic peptide as a yellow oil which was used in the next step without further purification. LC-MS (Method A, 1.5 min run) t_{R} 0.93 min, ESI+[M+H] calculated for C₇₁H₁₀₁N₁₂O₁₅S 1394.7; found 1394.8.

The fully protected cyclic peptide was subjected to the global deprotection conditions described in Method L, followed by preparative HPLC purification using Method O to afford the title compound, Example 9, as a white solid, 113 mg, 20%. ¹H NMR (400 MHz, d6-DMSO) δ 10.91 (br s, 1H), 8.70 (br s, 1H), 8.30 (s, 1H), 8.22 (d, J=6.0 Hz, 2H), 8.01 (d, J=6.5 Hz, 2H), 7.52-7.38 (m, 4H), 7.32 (d, J=8.0 Hz, 1H), 7.27-7.16 (m, 7H), 7.05 (t, J=7.5 Hz, 1H), 6.97 (t, J=7.3 Hz, 1H), 4.58-4.50 (m, 1H), 3.99 (br s, 1H), 3.64 (d, J=8.5 Hz, 5H), 3.53 (br s, 7H), 3.46 (br s, 7H), 3.42-3.33 (m, 8H), 3.28 (d, J=13.1 Hz, 5H), 3.21-3.07 (m, 6H), 3.04 (br s, 1H), 3.01-2.88 (m, 4H), 2.81-2.75 (m, 1H), 2.33 (br s, 4H), 2.06 (br s, 1H), 1.87 (d, J=8.5 Hz, 1H), 1.80 (s, 4H), 1.66 (br s, 2H), 1.48 (br s, 4H), 1.33 (br s, 4H), 1.25 (d, J=6.0 Hz, 2H), 1.14 (br s, 3H), 1.12-0.95 (m, 7H), 0.82 (br s, 3H). LC-MS (Method A, 1.5 min run) t_{R} 0.76 min, ESI+[M+H] calculated for C₄₉H₆₉N₁₂O₁₀ 986.1; found 986.4.

### EXAMPLE 10

### ((S)-1-(((3R,6S,9S,15S,19S,20aS)-6-((1H-indol-3-yl)methyl)-19-acetamido-9-(3-guanidinopropyl)-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method B, N^{α}-Fmoc-N^{δ}-allyloxycarbonyl-L-ornithine was loaded onto 2-chlorotrityl resin (CTC resin, 1.0 meq, 1.0 g, 1.0 mmol) and subsequently treated with piperidine in DMF (20% v/v) to afford the CTC resin-bound N^{δ}-allyloxycarbonyl-L-ornithine (0.6 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (342 mg, 0.9 mmol, 1.5 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (584 mg, 0.9 mmol, 1.5 equiv.), N^{α}-(((9H-fluoren-9-yl)methoxy)carbonyl)-1-(tert-butoxycarbonyl)-L-tryptophan (384 mg, 0.9 mmol, 1.5 equiv.), N-(9-fluorenylmethyloxycarbonyl)-3-(cis-4-hydroxycyclohexyl)-D-alanine (360 mg, 0.9 mmol, 1.5 equiv.) and (2S,4S)-1-(((9H-fluoren-9-yl)methoxy)carbonyl)-4-(acetylamino)pyrrolidine-2-carboxylic acid (406 mg, 0.9 mmol, 1.5 equiv.) to afford the resin-bound linear hexapeptide. This resin-bound product was subject to the cleavage conditions described in Method H to afford the linear hexapeptide intermediate as a crude oil, 847 mg, 100%. LC-MS (Method A, 1.5 min run) t_{R} 0.86 min, ESI+[M+H] calculated for C₇₁H₁₀₃N₁₂O₁₆S 1412.7; found 1412.5.

The linear hexapeptide (847 mg, 0.6 mmol, 1.0 equiv.) was subjected to the macrolactamization conditions described in Method J to afford the fully protected cyclic peptide as a yellow oil which was used in the next step without further purification. LC-MS (Method A, 1.5 min run) t_{R} 0.93 min, ESI+[M+H] calculated for C₇₁H₁₀₁N₁₂O₁₅S 1394.7; found 1394.8.

The fully protected cyclic peptide was subjected to the global deprotection condition described in Method L, followed by preparative HPLC purification using Method O to afford the title compound, Example 10, as a white solid, 130 mg, 30%. ¹H NMR (400 MHz, d6-DMSO) δ 10.88 (s, 1H), 8.61 (br s, 1H), 8.37 (br s, 1H), 8.16 (br s, 1H), 8.07 (d, J=6.5 Hz, 1H), 7.52 (d, J=8.0 Hz, 1H), 7.39-7.13 (m, 10H), 7.12-6.94 (m, 2H), 4.56 (br s, 1H), 4.41 (t, J=7.5 Hz, 1H), 4.29-3.98 (m, 5H), 3.93 (br s, 3H), 3.57 (br s, 8H), 3.46 (br s, 7H), 3.39 (d, J=5.5 Hz, 7H), 3.25 (d, J=15.6 Hz, 4H), 3.20-3.04 (m, 6H), 3.04-2.85 (m, 3H), 2.77 (dd, J=7.8, 14.3 Hz, 2H), 2.67 (br s, 1H), 2.27 (br s, 1H), 1.87 (br s, 1H), 1.80 (s, 3H), 1.69 (br s, 1H), 1.52 (br s, 2H), 1.46 (br s, 2H), 1.34 (br s, 3H), 1.23 (br s, 2H), 1.12 (br s, 5H), 0.94 (br s, 1H). LC-MS (Method A, 1.5 min run) t_{R} 0.76 min, ESI+[M+H] calculated for C₄₉H₆₉N₁₂O₁₀ 986.1; found 986.5.

### EXAMPLE 11

### N—((S)-1-(((3R,6S,9S,15S,19R,20aS)-6-((1H-indol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)-N-methylglycine

Following Method B, N^{α}-Fmoc-N^{δ}-allyloxycarbonyl-L-ornithine was loaded onto 2-chlorotrityl resin (CTC resin, 1.0 meq, 300 mg, 0.3 mmol) and subsequently treated with piperidine in DMF (20% v/v) to afford the CTC resin-bound N^{δ}-allyloxycarbonyl-L-ornithine (0.3 mmol).

The hexapeptide linear sequence was subsequently assembled by following the procedures described in Method F using sequentially N-(2-(tert-butoxy)-2-oxoethyl)-N-methyl-L-phenylalanine (132 mg, 0.45 mmol, 1.5 equiv), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (292 mg, 0.45 mmol, 1.5 equiv.), N^{α}-(((9H-fluoren-9-yl)methoxy)carbonyl)-1-(tert-butoxycarbonyl)-L-tryptophan (237 mg, 0.45 mmol, 1.5 equiv.), N-(9-fluorenylmethyloxycarbonyl)-3-(cis-4-hydroxycyclohexyl)-D-alanine (184 mg, 0.45 mmol, 1.5 equiv.) and (2S,4R)-1-(((9H-fluoren-9-yl)methoxy)carbonyl)-4-(tert-butoxy)pyrrolidine-2-carboxylic acid (184 mg, 1.5 equiv.) to afford the linear hexapeptide on resin. This resin-bound product was subjected to the cleavage conditions described in Method H to afford the linear hexapeptide as a white solid, 402 mg, crude yield 100%. LC-MS (Method A, 1.5 min run) t_{R} 0.81 min, ESI+[M+H] calculated for C₈₀H₁₁₈N₂O₂₀S 1341.7; found 1341.2.

The linear hexapeptide (402 mg, 0.3 mmol, 1.0 equiv.) was subjected to the macrolactamization conditions described in Method J to afford the fully protected cyclic peptide as a white solid, 194 mg, crude yield 100%. LC-MS (Method A, 1.5 min run) t_{R} 0.85 min, ESI+[M+H] calculated for C₆₉H₉₉N₁₁O₁₃S 1323.6; found 1323.5.

The crude fully protected cyclic peptide (194 mg, 0.3 mmol, 1.0 equiv.) was subjected to the global deprotection conditions described in Method L, which afforded the fully deprotected cyclic peptide as a white powder, 287 mg. This powder was purified by HPLC using Method O to afford the title compound, Example 11, as a white solid, 14.9 mg, 5.2%. ¹H NMR (400 MHz, d6-DMSO) δ 10.86 (s, 1H), 8.48 (d, J=6.0 Hz, 1H), 8.30 (s, 1H), 8.17 (br s, 1H), 7.83 (br s, 1H), 7.53 (d, J=7.5 Hz, 1H), 7.35-7.12 (m, 1H), 7.05 (t, J=7.0 Hz, 1H), 6.96 (t, J=7.5 Hz, 1H), 4.56-4.47 (m, 1H), 4.39 (br s, 1H), 4.26 (br s, 1H), 4.13-3.95 (m, 1H), 3.77-3.47 (m, 1H), 3.35 (br s, 46H), 3.20-2.91 (m, 2H), 2.89-2.60 (m, 1H), 2.52-2.48 (m, 40H), 2.36-2.30 (m, 1H), 2.24 (s, 1H), 1.97 (d, J=7.0 Hz, 1H), 1.85 (br s, 1H), 1.68-1.31 (m, 1H), 1.30-1.06 (m, 1H). LC-MS (Method A, 1.5 min run) t_{R} 0.51 min, ESI+[M+H] calculated for C₄₈H₆₈N₁₁O₁₀ 959.1; found 959.6.

### EXAMPLE 12

### ((31R,6S,9S,15S,19S,20aS)-6-((1H-indol-3-yl)methyl)-15-((S)-2-((carboxymethyl)amino)-3-phenylpropanamido)-9-(3-guanidinopropyl)-3-(((1s,4S)-4-hydroxycyclohexyl)methyl)-1,4,7,10,16-pentaoxoicosahydropyrrolo[1,2-a][1,4,7,10,13]pentaazacyclooctadecin-19-yl)glycine

Following Method B, N^{α}-Fmoc-N^{δ}-allyloxycarbonyl-L-ornithine was loaded onto 2-chlorotrityl resin (CTC resin, 1.0 meq, 200 mg, 0.2 mmol) and subsequently treated with piperidine in DMF (20% v/v) to afford the CTC resin-bound N^{δ}-allyloxycarbonyl-L-ornithine (0.2 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (114 mg, 0.3 mmol, 1.5 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (195 mg, 0.30 mmol, 1.5 equiv.), N^{α}-(((9H-fluoren-9-yl)methoxy)carbonyl)-1-(tert-butoxycarbonyl)-L-tryptophan (128 mg, 0.30 mmol, 1.5 equiv.), N-(9-fluorenylmethyloxycarbonyl)-3-(cis-4-hydroxycyclohexyl)-D-alanine (123 mg, 0.30 mmol, 1.5 equiv.) and (2S,4S)-1-(((9H-fluoren-9-yl)methoxy)carbonyl)-4-((2-(tert-butoxy)-2-oxoethyl)(tert-butoxycarbonyl)amino)pyrrolidine-2-carboxylic acid (70 mg, 0.15 mmol, 0.75 equiv.) to afford the resin-bound linear hexapeptide. This resin-bound product was subjected to the cleavage conditions described in Method H to afford the linear hexapeptide as a yellow oil, 297 mg, crude yield 100%. LC-MS (Method A) t_{R} 0.93 min, ESI+[M/2+H] calculated for C₇₅H₁₁₀N₁₂O₁₇S 742.9; found 742.5.

The crude linear hexapeptide (297 mg, 0.20 mmol, 1.0 equiv.) was subjected to the macrolactamization conditions described in Method J to afford the fully protected cyclic peptide as a yellow solid, 293 mg. This material was used in the global deprotection without further purification. LC-MS (Method A) t_{R} 1.01 min, ESI+[M/2+H] calculated for C₇₅H₁ₒ₈N₁₂O₁₆S 733.9; found 734.3.

The fully protected cyclic peptide (293 mg, 0.2 mmol) was subjected to the global deprotection conditions described in Method L, followed by preparative HPLC purification using Method O to afford the title compound, Example 12, as a white solid, 11.3 mg, 5.7%. ¹H NMR (400 MHz, d6-DMSO) δ 10.83 (s, 1H), 9.78 (br s, 1H), 8.30 (s, 1H), 8.23-8.05 (m, 2H), 7.75 (br s, 1H), 7.59 (br s, 1H), 7.48 (d, J=7.5 Hz, 1H), 7.31 (d, J=8.5 Hz, 1H), 7.26-7.13 (m, 10H), 7.10-7.01 (m, 3H), 6.96 (t, J=7.3 Hz, 2H), 4.69 (br s, 1H), 4.43 (br s, 1H), 4.31-4.10 (m, 5H), 4.06-3.88 (m, 3H), 3.59 (br s, 9H), 3.06 (d, J=16.1 Hz, 22H), 2.91-2.73 (m, 10H), 2.67 (br s, 9H), 1.40 (br s, 12H), 1.27-1.11 (m, 13H). LC-MS (Method A, 1.5 min run) t_{R} 0.50 min, ESI+[M+H] calculated for C₄₉H₆₈N₁₂O₁₁ 1002.1; found 1001.6.

### EXAMPLE 13

### ((S)-1-(((3R,6S,9S,15S,19R,20aS)-6-((1H-indol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-(3,5-dideuterophenyl)propan-2-yl)glycine

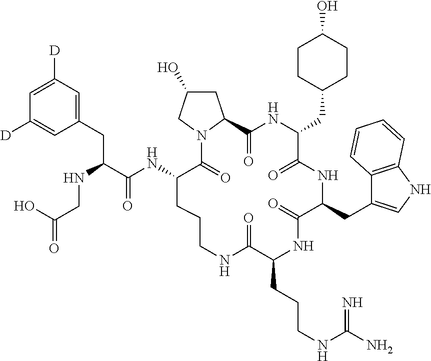 [SMILES: [2H]c1cc([2H])cc(C[C@H](NCC(=O)O)C(=O)N[C@H]2CCCNC(=O)[C@H](CCCNC(=N)N)NC(=O)[C@H](Cc3c[nH]c4ccccc34)NC(=O)[C@@H](C[C@H]3CC[C@@H](O)CC3)NC(=O)[C@@H]3C[C@@H](O)CN3C2=O)c1]

Following Method N, cyclic pentapeptide 1-(3-((3R,6S,9S,15S,19R,20aS)-6-((1H-indol-3-yl)methyl)-15-amino-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-9-yl)propyl)guanidine (200 mg, 0.27 mmol, 1.0 equiv.) was reacted with (S)-2-((2-(tert-butoxy)-2-oxoethyl)(tert-butoxycarbonyl)amino)-3-(3,5-dibromophenyl)propanoic acid (218 mg, 0.4 mmol, 1.5 equiv.) in the presence of HATU (162 mg, 0.43 mmol, 1.57 equiv.) and DIPEA (283 μL, 1.6 mmol, 6 equiv.) to afford hexapeptide tert-butyl N—((S)-1-(((3R,6S,9S,15S,19R,20aS)-6-((1H-indol-3-yl)methyl)-3-(((1s,4S)-4-(11-oxidanyl)cyclohexyl)methyl)-9-(3-guanidinopropyl)-19-(11-oxidanyl)-1,4,7,10,16-pentaoxoicosahydropyrrolo[1,2-a][1,4,7,10,13]pentaazacyclooctadecin-15-yl)amino)-3-(3,5-dibromophenyl)-1-oxopropan-2-yl)-N-(tert-butoxycarbonyl)glycinate as a yellow solid, 320 mg, 94% crude yield. LC-MS (Method A, 1.5 min run) t_{R} 0.87 min, ESI+[M+H] calculated for C₅₆H₇₉Br₂N₁₁O₁₂ 1259.1; found 1258.7.

This crude hexapeptide (80 mg, 0.064 mmol) was subjected to the deprotection conditions described in Method N, using HFIPA (3 ml) and treating with 12 M HCl (0.27 ml, 3.2 mmol, 50 equiv.) at ˜20° C. for 1 h to afford the crude deprotected product compound ((S)-1-(((3R,6S,9S,15S,19R,20aS)-6-((1H-indol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-3-(3,5-dibromophenyl)-1-oxopropan-2-yl)glycine as a yellow solid (100 mg, 100% crude yield). LC-MS (Method A, 1.5 min run) t_{R} 0.68 min, ESI+[M+H] calculated for C₄₇H₆₄Br₂N₁₁O₁₀ 1102.9; found 1103.1.

The crude deprotected hexapeptide (200 mg, 0.182 mmol, 1.0 equiv.) was placed In a 15 ml parr bottle with 10% Pd/C (19.3 mg, 0.182 mg, 1.0 equiv.) and MeOH. The reaction vessel was closed, degassed by vacuum/N₂ purge 10 times, followed by 3 cycles of vacuum/D₂ purge, after which the parr bottle was charged with D₂ gas to 15 psi and stirred at 18° C. for 2 h. The vessel was refilled with D₂ gas and was allowed to stir at r.t. for another 16 h. The mixture was then filtered through a pad of celite, and the filtrate evaporated to dryness to afford a yellow oil, which was purified by preparative HPLC using Method O to afford the title compound, Example 13, as a white solid, 36.7 mg, 21.4%. ¹H NMR (400 MHz, d6-DMSO) δ 10.91 (br s, 1H), 8.58 (br s, 1H), 8.45 (br s, 1H), 8.37-8.27 (m, 1H), 8.10-7.85 (m, 1H), 7.52 (s, 1H), 7.32 (d, J=8.5 Hz, 1H), 7.27-7.15 (m, 3H), 7.12-6.91 (m, 2H), 4.69-4.55 (m, 1H), 4.61 (d, J=7.5 Hz, 4H), 4.42 (br s, 1H), 4.22 (br s, 2H), 4.08 (br s, 2H), 3.98-3.83 (m, 1H), 3.91 (br s, 5H), 3.39 (br s, 71H), 3.13-2.86 (m, 3H), 2.78 (s, 1H), 2.67 (s, 1H), 2.52-2.49 (m, 97H), 2.05-1.80 (m, 1H), 1.70-1.44 (m, 1H), 1.50 (br s, 3H), 1.08 (d, J=10.5 Hz, 4H), 1.42-0.92 (m, 5H). HRMS (m/z) [M+H]+ calculated for C₄₇H₆₄D₂N₁₁O₁₀ 946.5114, found 946.5095.

### EXAMPLE 14

### ((31R,6S,9S,15S,19R,20aS)-6-((1H-indol-3-yl)methyl)-15-((S)-2-((carboxymethyl)amino)-3-phenylpropanamido)-9-(3-guanidinopropyl)-3-(((1s,4S)-4-hydroxycyclohexyl)methyl)-1,4,7,10,16-pentaoxoicosahydropyrrolo[1,2-a][1,4,7,10,13]pentaazacyclooctadecin-19-yl)glycine

Following Method B, N^{α}-Fmoc-N^{δ}-allyloxycarbonyl-L-ornithine was loaded onto 2-chlorotrityl resin (CTC resin, 1.0 meq, 400 mg, 0.4 mmol) and subsequently treated with piperidine in DMF (20% v/v) to afford the CTC resin-bound N^{δ}-allyloxycarbonyl-L-ornithine (0.4 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (230 mg, 0.6 mmol, 1.5 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (390 mg, 0.60 mmol, 1.5 equiv.), N^{α}-(((9H-fluoren-9-yl)methoxy)carbonyl)-1-(tert-butoxycarbonyl)-L-tryptophan (260 mg, 0.60 mmol, 1.5 equiv.), N-(9-fluorenylmethyloxycarbonyl)-3-(cis-4-hydroxycyclohexyl)-D-alanine (252 mg, 0.60 mmol, 1.5 equiv.) and (2S,4R)-1-(((9H-fluoren-9-yl)methoxy)carbonyl)-4-((2-tert-butoxy-2-oxoethyl)(tert-butoxycarbonyl)amino)pyrrolidine-2-carboxylic acid (135 mg, 0.24 mmol, 0.60 equiv.) to afford the resin-bound linear hexapeptide. This resin-bound product was subject to cleavage conditions described in Method H to afford the linear hexapeptide as a solid which was used for the next step without further purification. LC-MS (Method A, 1.5 min run) t_{R} 0.96 min, ESI+[(M-tBu)/2+H] calculated for C₇₅H₁₁₀N₁₂O₁₇S 714.1; found 714.5.

The linear hexapeptide (0.40 mmol, 1.0 equiv.) was subjected to macrolactamization conditions described in Method J to afford the fully protected cyclic peptide as a yellow solid, 586 mg. This material was used in the subsequent global deprotection without further purification. LC-MS (Method A, 1.5 min run) t_{R} 1.06 min, ESI+[(M-tBu-tBu-Boc)/2+H] calculated for C₇₅H₁ₒ₈N₁₂O₁₆S 627.9; found 627.5.

The fully protected cyclic peptide (293 mg, 0.2 mmol) was subjected to the global deprotection conditions described in Method M, followed by preparative HPLC purification using Method O to afford the title compound, Example 14, as a white solid, 38.9 mg, 19.4%. ¹H NMR (400 MHz, d6-DMSO) δ 10.86 (br s, 1H), 9.99 (br s, 1H), 8.62 (br s, 1H), 8.37 (br s, 1H), 8.24 (s, 1H), 7.88 (d, J=6.5 Hz, 1H), 7.49 (d, J=8.0 Hz, 1H), 7.31 (d, J=8.0 Hz, 1H), 7.28-7.18 (m, 3H), 7.09-6.95 (m, 1H), 6.58 (br s, 1H), 4.74 (d, J=8.5 Hz, 1H), 4.56 (br s, 1H), 4.23 (br s, 1H), 4.12 (br s, 1H), 4.04 (br s, 1H), 3.83 (br s, 3H), 3.34 (br s, 6H), 3.25-2.94 (m, 7H), 2.94-2.85 (m, 1H), 2.81 (d, J=8.0 Hz, 1H), 2.67 (br s, 1H), 2.50 (br s, 52H), 2.33 (s, 1H), 2.07 (br s, 1H), 1.93-1.71 (m, 1H), 1.64 (br s, 1H), 1.53 (br s, 1H), 1.29 (br s, 2H), 1.21 (br s, 1H), 1.14 (br s, 1H), 1.00 (d, J=19.6 Hz, 2H). LC-MS (Method A, 1.5 min run) t_{R} 0.78 min, ESI+[M+H] calculated for C₄₉H₆₈N₁₂O₁₁ 1002.1; found 1002.3.

### EXAMPLE 15

### ((S)-1-(((3R,6S,9S,15S,19R,20aS)-9-(3-guanidinopropyl)-19-hydroxy-3-(((1s,4S)-4-hydroxycyclohexyl)methyl)-6-((6-methoxy-1H-indol-3-yl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method B, N^{α}-Fmoc-N^{δ}-allyloxycarbonyl-L-ornithine was loaded onto 2-chlorotrityl resin (CTC resin, 1.0 meq, 400 mg, 0.4 mmol) and subsequently treated with piperidine in DMF (20% v/v) to afford the CTC resin-bound N^{δ}-allyloxycarbonyl-L-ornithine (0.4 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (230 mg, 0.6 mmol, 1.5 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (390 mg, 0.60 mmol, 1.5 equiv.), (S)-2-((((9H-fluoren-9-yl)methoxy)carbonyl)amino)-3-(6-methoxy-1H-indol-3-yl)propanoic acid (276 mg, 0.60 mmol, 1.5 equiv.), N-(9-fluorenylmethyloxycarbonyl)-3-(cis-4-hydroxycyclohexyl)-D-alanine (252 mg, 0.60 mmol, 1.5 equiv.) and (2S,4R)-1-(((9H-fluoren-9-yl)methoxy)carbonyl)-4-(tert-butoxy)pyrrolidine-2-carboxylic acid (246 mg, 0.6 mmol, 1.50 equiv.) to afford the resin-bound linear hexapeptide. This resin-bound product was subjected to the cleavage conditions described in Method H to afford the linear hexapeptide as a solid which was used for the next step without further purification. LC-MS (Method A, 1.5 min run) t_{R} 0.96 min, ESI+[M+H] calculated for C₇₄H₁₀₉N₁₁O₁₇S 1457.8; found 1457.5.

The linear hexapeptide (0.20 mmol, 1.0 equiv.) was subjected to the macrolactamization conditions described in Method J to afford the fully protected cyclic peptide as a yellow solid, 288 mg. This material was used in the subsequent global deprotection without further purification. LC-MS (Method A, 1.5 min run) t_{R} 0.99 min, ESI+[(M-tBu-tBu-Boc)/2+H] calculated for C₇₄H₁ₒ₇N₁₁O₁₆S 613.9; found 613.8.

The fully protected cyclic peptide (293 mg, 0.2 mmol) was subject to the global deprotection conditions described in Method M, followed by preparative HPLC purification using Method O to afford the title compound, Example 15, as a white solid, 4.5 mg, 2.3%. ¹H NMR (400 MHz, d6-DMSO) δ 10.75 (d, J=13.1 Hz, 1H), 8.53 (br s, 3H), 8.41 (s, 5H), 8.23-7.80 (m, 6H), 7.53-7.33 (m, 14H), 7.29-7.16 (m, 21H), 7.09-6.97 (m, 2H), 6.82 (d, J=8.5 Hz, 1H), 6.63 (d, J=8.0 Hz, 1H), 5.75 (s, 6H), 4.60 (br s, 8H), 4.41 (br s, 8H), 4.29-3.99 (m, 23H), 3.83-3.71 (m, 20H), 3.68 (br s, 17H), 3.17 (s, 21H), 3.12-2.84 (m, 4H), 2.52-2.48 (m, 163H), 2.41-2.31 (m, 30H), 2.07-1.77 (m, 6H), 1.52 (br s, 14H), 1.36 (br s, 17H), 1.24 (br s, 15H), 1.19-1.05 (m, 26H). LC-MS (Method A, 1.5 min run) t_{R} 0.51 min, ESI+[M+H] calculated for C₄₈H₆₇N₁₁O₁₁ 975.1; found 975.5.

### EXAMPLE 16

### ((S)-1-(((31R,6S,9S,15S,19R,20aS)-6-((1H-indazol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method B, N^{α}-Fmoc-N^{δ}-allyloxycarbonyl-L-ornithine was loaded onto 2-chlorotrityl resin (CTC resin, 1.0 meq, 600 mg, 0.6 mmol) and subsequently treated with piperidine in DMF (20% v/v) to afford the CTC resin-bound N^{δ}-allyloxycarbonyl-L-ornithine (0.6 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (227 mg, 0.60 mmol, 1.0 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (571 mg, 0.9 mmol, 1.5 equiv.), (S)-2-((((9H-fluoren-9-yl)methoxy)carbonyl)amino)-3-(1-(tert-butoxycarbonyl)-1H-indazol-3-yl)propanoic acid (317 mg, 0.60 mmol, 1.0 equiv.), N-(9-Fluorenylmethyloxycarbonyl)-3-(cis-4-hydroxycyclohexyl)-D-alanine (122 mg, 0.300 mmol, 0.5 equiv.), (2S,4R)-1-(((9H-fluoren-9-yl)methoxy)carbonyl)-4-(tert-butoxy)pyrrolidine-2-carboxylic acid (369 mg, 0.90 mmol, 1.5 equiv.) to afford the resin-bound linear hexapeptide. This resin-bound product was subject to cleavage conditions described in Method H to afford the crude linear hexapeptide intermediate, which was purified by reverse phase flash chromatography (Spherical 20*45 mm column (C18, 100 A, 26 g), gradient acetonitrile/water 10% 5 min to 90% in 10 min then 100% MeCN in 6 min, 35 mL/min) to afford the desired linear peptide as a white solid, 100 mg, yield 10%. LC-MS (Method A, 1.5 min run) t_{R} 0.926 min, ESI+[M/2+H] calculated for C₇₇H₁₁₆N₁₂O₁₈S 764.4; found 765.0.

The crude linear hexapeptide (90 mg, 0.059 mmol, 1.0 equiv.) was subject to the macrolactamization conditions described in Method J to afford the fully protected cyclic peptide as an off-white solid, which was used in the global deprotection without further purification.

The crude fully protected cyclic peptide was subject to global deprotection condition described in Method L, followed by preparative HPLC purification using Method O to afford the zwitterionic form of the title compound, Example 16, as a white solid, 20.7 mg, 35%. ¹H NMR (400 MHz, d6-DMSO) δ 13.25-12.93 (m, 1H), 9.19-8.33 (m, 1H), 7.71 (d, J=8.0 Hz, 1H), 7.62-7.38 (m, 1H), 7.34-7.14 (m, 1H), 7.11-6.98 (m, 1H), 4.58 (br. s., 1H), 4.10 (d, J=3.0 Hz, 1H), 3.83 (br. s., 1H), 3.74-3.51 (m, 1H), 3.47 (br. s., 1H), 3.39-3.32 (m, 17H), 3.05-2.85 (m, 1H), 2.82-2.59 (m, 1H), 2.54-2.49 (m, 13H), 1.63-1.44 (m, 1H), 1.39 (br. s., 1H), 1.25 (br. s., 1H), 1.20-1.00 (m, 1H), 0.89 (br. s., 1H). LC-MS (Method A, 1.5 min run) t_{R} 0.63 min, ESI+[M+H] calculated for C₄₆H₆₅N₁₂O₁₀ 946.1; found 946.6.

### EXAMPLE 17

### ((S)-1-(((31R,6S,9S,15S,19R,20aS)-6-((6-ethyl-1H-indol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine

Following Method B, N^{α}-Fmoc-N^{δ}-allyloxycarbonyl-L-ornithine was loaded onto 2-chlorotrityl resin (CTC resin, 1.0 meq, 600 mg, 0.6 mmol) and subsequently treated with piperidine in DMF (20% v/v) to afford the CTC resin-bound N^{δ}-allyloxycarbonyl-L-ornithine (0.6 mmol).

The hexapeptide linear sequence was subsequently assembled by following the procedures described in Method F using sequentially N-(2-(tert-butoxy)-2-oxoethyl)-N-(tert-butoxycarbonyl)-L-phenylalanine (227 mg, 0.60 mmol, 1.0 equiv.), N^{α}-(((9H-fluoren-9-yl)methoxy)carbonyl)-N^{ω}-((2,2,4,6,7-pentamethyl-2,3-dihydrobenzofuran-5-yl)sulfonyl)-L-arginine (571 mg, 0.9 mmol, 1.5 equiv.), (S)-2-((((9H-fluoren-9-yl)methoxy)carbonyl)amino)-3-(1-(tert-butoxycarbonyl)-6-ethyl-1H-indol-3-yl)propanoic acid (333 mg, 0.60 mmol, 1.0 equiv.), N-(9-Fluorenylmethyloxycarbonyl)-3-(cis-4-hydroxycyclohexyl)-D-alanine (409 mg, 0.90 mmol, 1.5 equiv.), (2S,4R)-1-(((9H-fluoren-9-yl)methoxy)carbonyl)-4-(tert-butoxy)pyrrolidine-2-carboxylic acid (369 mg, 0.90 mmol, 1.5 equiv.) to afford the resin-bound linear hexapeptide. This resin-bound product was subject to cleavage conditions described in Method H to afford the crude linear hexapeptide intermediate, 470 mg, crude yield 50%. LC-MS (Method A, 1.5 min run) t_{R} 0.96 min, ESI+[M/2+H] calculated for C₈₀H₁₂₁N₁₁O₁₈S 777.9; found 778.3.

The crude linear hexapeptide (470 mg, 0.302 mmol, 1.0 equiv.) was subject to the macrolactamization conditions described in Method J to afford the crude fully protected cyclic peptide as an yellow solid, which was purified by reverse phase flash chromatography (Spherical 20*45 mm column (C18, 100 A, 26 g), gradient acetonitrile/water 10% 5 min to 90% in 10 min then 100% MeCN in 6 min, 35 mL/min) to afford the cyclic peptide as a white solid, 195 mg, yield 42%.

The purified fully protected cyclic peptide was subject to global deprotection condition described in Method L, followed by preparative HPLC purification using Method O to afford the zwitterionic form of the title compound, Example 17, as a white solid, 23.8 mg, 19%. ¹H NMR (400 MHz, d6-DMSO) δ 10.91-10.63 (m, 1H), 8.97 (br. s., 1H), 8.69-8.22 (m, 1H), 8.10-7.78 (m, 1H), 7.74-7.34 (m, 1H), 7.27-7.17 (m, 1H), 7.11 (d, J=11.5 Hz, 1H), 3.55 (br. s., 1H), 3.37 (br. s., 20H), 3.08 (br. s., 1H), 3.04-2.90 (m, 1H), 2.80-2.72 (m, 1H), 2.70-2.58 (m, 1H), 2.54-2.49 (m, 13H), 1.85 (br. s., 1H), 1.60 (br. s., 1H), 1.51 (br. s., 1H), 1.47-1.29 (m, 1H), 1.29-1.01 (m, 3H). LC-MS (Method A, 1.5 min run) t_{R} 0.63 min, ESI+[M+H] calculated for C₄₉H₆₉N₁₁O₁₀ 972.5.1; found 973.1.

### Preparation 1

### N-(9-Fluorenylmethyloxycarbonyl)-3-(cis-4-hydroxycyclohexyl)-D-alanine

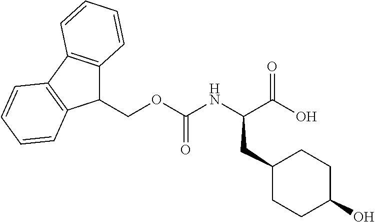 [SMILES: O=C(N[C@H](C[C@H]1CC[C@@H](O)CC1)C(=O)O)OCC1c2ccccc2-c2ccccc21]

### Step 1

### Preparation of (R)-methyl 2-amino-3-(4-hydroxyphenyl)propanoate

To a 2000 ml round bottom flask equipped with a stir bar was added (R)-3-(4-hydroxyphenyl)alanine (150.0 g, 828 mmol, 1.00 eq) followed by methanol (1000 ml). The resulting suspension was cooled in an ice-water bath. To the suspension was added SOCl₂ (148 g, 1240 mmol, 1.50 eq) drop-wise. The solution was warmed to 10° C. and then refluxed at 85° C. for 12 hrs. The resulting solution was concentrated under reduced pressure to provide 130 g of the title compound as a solid, which was taken to the next step without any further purification.

### Step 2

### Preparation of (R)-methyl 2-(tert-butoxycarbonylamino)-3-(4-hydroxyphenyl)propanoate

To a 3 L round bottom flask was added crude (R)-methyl 2-amino-3-(4-hydroxyphenyl)propanoate (202 g, 871.8 mmol, 1.00 eq), triethylamine (221 g, 2.2 mol, 2.50 eq) and DCM (1 L) to give a slurry. The slurry was cooled in an ice bath and a solution of (Boc)₂O (209 g, 959 mmol, 1.10 eq) in DCM (1 L) was added drop-wise. After the addition, the resulting colorless solution was warmed to 10° C. and stirred for 18 hrs. To the mixture was added water (1.5 L), followed by the slow addition of concentrated HCl until pH 3 was achieved. The organic phase was separated, washed with diluted HCl (0.2 mol/L, 1.5 L) and brine (1.5 L×2). The organic phase was dried over anhydrous Na₂SO₄, filtered and concentrated under reduced pressure to provide crude product. To the crude product was added DCM (400 ml) and PE (2 L) to give a white suspension. The suspension was stirred at 10° C. for 1 h then filtered. The resulting filter cake was dried under vacuum to provide 210 g of the title compound as a solid, which was taken to the next step without any further purification.

### Step 3

### Preparation of (2R)-methyl 2-(tert-butoxycarbonylamino)-3-(4-hydroxycyclohexyl)propanoate (Mixture of Cis and Trans Isomers)

To each of two 2 L hydrogenation vessels was added about 105 g of (R)-methyl 2-(tert-butoxycarbonylamino)-3-(4-hydroxyphenyl)propanoate (355 mmol, 1.00 eq), followed by methanol (3 L) and Rh/C (36 g, 0.05 eq, 5% on wet carbon). The black suspensions were evacuated under vacuum and refilled with H₂ (3×). The resulting reaction mixtures were stirred at 50 psi of hydrogen pressure at 50° C. for 24 hours. The mixtures were filtered and the filtrates combined and concentrated under reduced pressure to a crude oil (˜220 g). The crude product was suspended in EtOAc (200 ml) and PE (1 L) and was stirred at 10° C. for 30 min then filtered. The filter cake was dried under vacuum to provide 84 g of the title compound as a solid. The solid was a mixture of both cis and trans isomers across the cyclohexyl group that was carried through subsequent steps until final isolation of the desired cis isomer as described in step 7.

### Step 4

### Preparation of (2R)-2-(tert-butoxycarbonylamino)-3-(4-hydroxycyclohexyl)propanoic Acid (Mixture of Cis and Trans Isomers)

To a 2 L round bottom flask were added (2R)-methyl 2-(tert-butoxycarbonylamino)-3-(4-hydroxycyclohexyl)propanoate (84 g, 278.7 mmol, 1.0 eq.), THF (500 ml), water (500 ml) and LiOH—H₂O (23.4 g, 557 mmol, 2.0 eq.). The solution was stirred at 10° C. for 2 hrs. Most of the THF was removed under reduced pressure and the residue acidified to pH ˜5 with the addition of dilute aqueous 1 M HCl. The mixture was extracted with DCM (800 ml) and EtOAc (500 ml×2). The combined organic phase was dried over anhydrous Na₂SO₄, filtered and concentrated under reduced pressure to provide 80 g of the title compound as a white solid, which was used in the next step without purification.

### Step 5

### Preparation of (2R)-2-amino-3-(4-hydroxycyclohexyl)propanoic Acid (Mixture of Cis and Trans Isomers)

To a 2 L round bottom flask was added (2R)-2-(tert-butoxycarbonylamino)-3-(4-hydroxycyclohexyl)propanoic acid (80.0 g, 278.40 mmol, 1.0 eq.) followed by EtOAc (500 ml). To the suspension at 15° C. was added a solution of HCl in EtOAc (˜4 M, 500 ml, 2 mol, 7.18 eq). The reaction mixture was stirred at 15° C. for 1 h. The mixture was filtered and the filter cake was dried under vacuum to provide 62 g of the title compound as a white solid, which was used in the next step directly.

### Step 6

### Preparation of (2R)-2-((((9H-fluoren-9-yl)methoxy)carbonyl)amino)-3-(4-hydroxycyclohexyl)propanoic Acid (Mixture of Cis and Trans Isomers)

To a 2 L round bottom flask were added (2R)-2-amino-3-(4-hydroxycyclohexyl)propanoic acid (62.0 g, 277.11 mmol, 1.0 eq), 500 ml of H₂O and 500 ml of dioxane followed by Na₂CO₃ (88.1 g, 831 mmol, 3.00 eq). The suspension was stirred at 15° C. for 10 min then placed in an ice-water bath. To the reaction mixture in the ice bath was added solid Fmoc-OSu (93.5 g, 277 mmol, 1.0 eq) in portions. After 30 min, the ice-bath was removed and the mixture was stirred for 90 minutes. The mixture was diluted with 500 ml of water and adjusted to pH 4 with the addition of saturated aqueous citric acid solution. The mixture was extracted with DCM (800 ml×2). The combined organic phase was washed with 500 ml of brine and dried over anhydrous Na₂SO₄. The solution was filtered and concentrated to 120 g of the title compound, also known as N-(9-fluorenylmethyloxycarbonyl)-3-(4-hydroxycyclohexyl)-D-alanine, as a yellow oil.

### Step 7

### Isolation of N-(9-Fluorenylmethyloxycarbonyl)-3-(cis-4-hydroxycyclohexyl)-D-alanine

Isolation of the title compound from the mixture of cis and trans isomers of N-(9-fluorenylmethyloxycarbonyl)-3-(4-hydroxycyclohexyl)-D-alanine from Step 6 (120 g) was achieved by preparative HPLC using a Phenomenex C18 250×80 mm×10 μm column with an eluent of acetonitrile in water, starting with 40% acetonitrile and increasing to 60% acetonitrile over 27 minutes and a flow rate of 250 ml/min. This procedure provided 62 g of the title compound as a solid. ¹H NMR (400 MHz, CD₃OD) δ 7.79-7.77 (d, 2H), 7.69-7.59 (m, 2H), 7.44-7.33 (m, 2H), 7.32-7.28 (m, 2H), 4.42-4.30 (m, 2H), 4.24-4.20 (m, 1.9H), 4.03-3.98 (m, 0.1H), 3.89-3.86 (m, 1H), 1.78-1.39 (m, 11H); LCMS: MS=432.0 (M+Na). HPLC retention time=4.31 min. Column: Ultimate XB-C18, 3 μm, 3.0×50 mm. Mobile Phase: 1.0% MeCN in water (0.1% TFA) to 5% MeCN in water (0.1% TFA) in 1 min; then gradient to 100% MeCN in 5 minutes; hold at 100% MeCN for 2 minutes; back to 1.0% MeCN in water (0.1% TFA) at 8.01 minutes and hold two minutes. Flow rate: 1.2 ml/min.

### Preparation 2

### N-(2-(tert-butoxy)-2-oxoethyl)-N-(tert-butoxycarbonyl)-L-phenylalanine

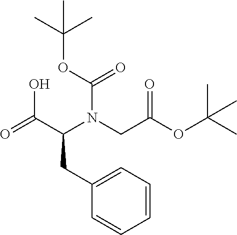 [SMILES: CC(C)(C)OC(=O)CN(C(=O)OC(C)(C)C)[C@@H](Cc1ccccc1)C(=O)O]

### Step 1

### Preparation of (S)-benzyl 2-(2-tert-butoxy-2-oxoethylamino)-3-phenylpropanoate

To a 3 L round bottom flask were added benzyl L-phenylalaninate hydrochloride (200.0 g, 609.3 mmol, 1.00 eq.), DMF (2.0 L), and K₂CO₃ (168.0 g, 1220 mmol, 2.00 eq.). The slurry was stirred at 20° C. for 30 minutes. To the reaction mixture was added drop-wise neat tert-butyl 2-bromoacetate (131.0 g, 670 mmol, 1.10 eq). After the addition, the mixture was heated to 50° C. and was stirred for 4 hours. The reaction was cooled to 20° C. and diluted with EtOAc (4.0 L), then the resulting mixture was washed with water (6.0 L×3), and the organic layer dried over MgSO₄, filtered and concentrated under reduced pressure to provide an off-white solid. The solid was purified via silica-gel column chromatography (eluent: petroleum ether/ethyl acetate from 100/0 to 0/100) to give an off-white solid. To the solid was added PE (1.5 L) and the resulting white suspension was vigorously stirred at room temperature for 1 hour and then filtered. The filtrate was concentrated under reduced pressure to provide 114 g of the title compound as a yellow oil. LCMS: m/z 391.9 (M+Na⁺). HPLC retention time: 4.03 min. Column: Ultimate XB-C18, 3 μm, 3.0×50 mm. Mobile Phase: 1.0% MeCN in water (0.1% TFA) to 5% MeCN in water (0.1% TFA) in 1 min; then gradient to 100% MeCN in 5 minutes; hold at 100% MeCN for 2 minutes; back to 1.0% MeCN in water (0.1% TFA) at 8.01 minutes and hold two minutes. Flow rate: 1.2 ml/min.

### Step 2

### Preparation of (S)-benzyl 2-((2-tert-butoxy-2-oxoethyl)(tert-butoxycarbonyl)amino)-3-phenylpropanoate

To a 1 L round bottom flask were added (S)-benzyl 2-(2-tert-butoxy-2-oxoethylamino)-3-phenylpropanoate (20.0 g, 54.1 mmol, 1.00 eq.) and (Boc)₂O (118 g, 541 mmol, 10.0 eq.) and the resulting mixture heated to 70° C. To the reaction was carefully added solid DMAP (19.8 g, 162 mmol, 3.00 eq.). The reaction was stirred at 70° C. for 10 minutes. Additional (Boc)₂O (177 g, 809 mmol, 15.0 eq.) was added in portions over a period of 2 h. The resulting crude mixture was combined with a second batch of crude (S)-benzyl 2-((2-tert-butoxy-2-oxoethyl)(tert-butoxycarbonyl)amino)-3-phenylpropanoate prepared the same way. Purification of the combined batch by silica-gel column chromatography (eluent petroleum ether/ethyl acetate from 100/0 to 100/5) provided 15.5 g of the title compound as an oil. LCMS: m/z 492.3 (M+Na⁺). HPLC retention time=5.83 min. Column: Ultimate XB-C18, 3 μm, 3.0×50 mm. Mobile Phase: 1.0% MeCN in water (0.1% TFA) to 5% MeCN in water (0.1% TFA) in 1 min; then gradient to 100% MeCN in 5 minutes; hold at 100% MeCN for 2 minutes; back to 1.0% MeCN in water (0.1% TFA) at 8.01 minutes and hold two minutes. Flow rate: 1.2 ml/min. Optical Rotation: −69.248, in MeOH, c=0.133 g/ml.

### Step 3

### Preparation of N-(2-(tert-butoxy)-2-oxoethyl)-N-(tert-butoxycarbonyl)-L-phenylalanine

(S)-benzyl 2-((2-tert-butoxy-2-oxoethyl)(tert-butoxycarbonyl)amino)-3-phenylpropanoate (17.8 g, 37.9 mmol, 1.00 eq.) was added to a solvent mixture of THF (150 ml) and MeOH (150 ml). To the reaction was added dry Pd/C (2.02 g, 10% w.t., 1.90 mmol, 0.05 eq.). The resulting suspension was evacuated under vacuum and refilled with H₂ (3×), and then stirred under hydrogen pressure of 50 Psi for 12 h at 20° C. The reaction mixture was filtered and the filtrate concentrated under reduced pressure to provide 12.7 g of the title compound as a solid. ¹H NMR (400 MHz, CDCl₃) δ 7.34-7.24 (m, 3H), 7.16-7.11 (m, 2H), 4.19-4.14 (d, 0.75H), 3.86-3.81 (m, 0.5H), 3.68-3.64 (m, 0.73H), 3.39-3.19 (m, 2H), 2.88-2.83 (d, 0.27H), 2.58-2.53 (d, 0.75H), 1.53-1.50 (m, 8H), 1.45-1.43 (m, 10H). HPLC retention time=4.87 min. Column: Ultimate XB-C18, 3 μm, 3.0×50 mm. Mobile Phase: 1.0% MeCN in water (0.1% TFA) to 5% MeCN in water (0.1% TFA) in 1 min; then gradient to 100% MeCN in 5 minutes; hold at 100% MeCN for 2 minutes; back to 1.0% MeCN in water (0.1% TFA) at 8.01 minutes and hold two minutes. Flow rate: 1.2 ml/min. SFC chiral column retention time=2.21 min. Column: Chiralcel OD-3 150×4.6 mm I.D., 3 um; Mobile phase: A: supercritical CO₂; B: ethanol (0.05% DEA); Gradient: from 5% to 40% of B in 5 min and hold 40% for 2.5 min, then 5% of B for 2.5 min; Flow rate: 2.5 ml/min; Column temperature: 35° C.

### Preparation 3

### (S)-2-(((9H-fluoren-9-yl)methoxy)carbonylamino)-3-(4-methyl-1H-pyrazol-1-yl)propanoic Acid

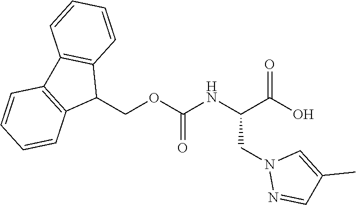 [SMILES: Cc1cnn(C[C@H](NC(=O)OCC2c3ccccc3-c3ccccc32)C(=O)O)c1]

### Step 1

### Preparation of (S)-2-(tert-butoxycarbonylamino)-3-(4-methyl-1H-pyrazol-1-yl)propanoic Acid

To a 50 ml round bottom flask were added (S)-tert-butyl 2-oxooxetan-3-ylcarbamate (1870 mg, 9.99 mmol, 1.0 eq.), 4-methyl-1H-pyrazole (984 mg, 12 mmol, 1.2 eq.) and MeCN (30 ml). The resulting solution was heated to 50° C. for 16 hours. The mixture was concentrated under reduced pressure to a light yellow residue. The residue was dissolved in hot MeOH (5 ml). After the reaction was cooled, a white solid precipitated from the solution. To the resulting mother liquor was added water (1 ml×3) to precipitate more solid. All solids were combined to provide 1.8 g of the title compound which was used in the next step without purification.

### Step 2

### Preparation of (S)-2-amino-3-(4-methyl-1H-pyrazol-1-yl)propanoic Acid Hydrochloride

To a mixture of (S)-2-(tert-butoxycarbonylamino)-3-(4-methyl-1H-pyrazol-1-yl)propanoic acid (1.8 g, 6.57 mmol, 1.0 eq.) and 1,4-dioxane (30.0 ml) was added a saturated solution of HCl in 1,4-dioxane (10.0 ml). The mixture was stirred at 25° C. for 1 hour. The mixture was concentrated under reduced pressure to provide 1.1 g of title compound as an oil, which was used in the next step without purification.

### Step 3

### Preparation of (S)-2-(((9H-fluoren-9-yl)methoxy)carbonylamino)-3-(4-methyl-1H-pyrazol-1-yl)propanoic Acid

A mixture of (S)-2-amino-3-(4-methyl-1H-pyrazol-1-yl)propanoic acid hydrochloride (1100 mg, 6.57 mmol, 1.0 eq.), dioxane (20 ml), water (5 ml) and Na₂CO₃ (1040 mg, 9.86 mmol, 1.5 eq.) was cooled in an ice bath and neat Fmoc-O-Su (2220 mg, 6.57 mmol, 1.0 eq.) was added. The reaction mixture was warmed to 25° C. and was stirred for one more hour. The mixture was diluted with water (60 ml), acidified to pH˜4 with the addition of acetic acid and was extracted with DCM (50 ml×3). The combined DCM layers were dried over anhydrous Na₂SO₄, filtered and concentrated under reduced pressure. The residue was purified by flash chromatography to afford 1.5 g of an off-white solid. The solid was further purified by preparative HPLC (Column: Phenomenex Synergi Max-RP 250*80 10 u; Mobile phase: from 35% MeCN in water (0.2% FA) to 65% MeCN in water (0.2% FA); Flow rate: 80 ml/min; Wavelength: 220 nm) to provide 1.0 g of the title compound as a white solid.

¹H NMR (400 MHz, CDCl₃) δ 7.80-7.78 (d, 2H), 7.62-7.60 (d, 2H), 7.45-7.31 (m, 5H), 6.96 (s, 1H), 5.71-5.70 (d, 1H), 4.74-4.38 (m, 5H), 4.26-4.22 (dd, 1H), 2.0 (s, 1H); LCMS: m/z 392.2 (M+H⁺)

### Preparation 4

### (2S,4R)-1-(((9H-fluoren-9-yl)methoxy)carbonyl)-4-(acetylamino)pyrrolidine-2-carboxylic Acid

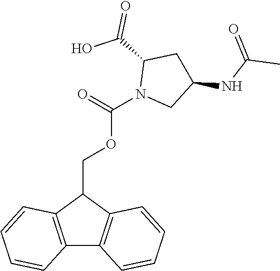 [SMILES: CC(=O)N[C@@H]1C[C@@H](C(=O)O)N(C(=O)OCC2c3ccccc3-c3ccccc32)C1]

### Step 1

### Preparation of (2S,4R)-1-(9H-fluoren-9-yl)methyl 2-methyl 4-aminopyrrolidine-1,2-dicarboxylate Hydrochloride

(2S,4R)-1-(((9H-fluoren-9-yl)methoxy)carbonyl)-4-(tert-butoxycarbonylamino)pyrrolidine-2-carboxylic acid (1.00 g, 2.210 mmol, 1.00 eq) was dissolved in MeOH (40 ml). The reaction mixture was placed in an ice-bath followed by the drop-wise addition of neat SOCl₂ (526 mg, 4.42 mmol, 2.00 eq). After the addition, the resulting reaction mixture was stirred at 70° C. for 16 h. The mixture was concentrated under reduced pressure to provide 890 mg of the title compound as a solid. The crude material was taken to the next step without any further purification.

### Step 2

### Preparation of (2S,4R)-1-(9H-fluoren-9-yl)methyl 2-methyl 4-acetamidopyrrolidine-1,2-dicarboxylate

To a solution of crude (2S,4R)-1-(9H-fluoren-9-yl)methyl 2-methyl 4-aminopyrrolidine-1,2-dicarboxylate hydrochloride (890 mg, 2.21 mmol, 1.00 eq) in DCM (80 ml) was added Na₂CO₃ (702 mg, 6.63 mmol, 3.00 eq). The mixture was stirred for 5 min then placed in an ice-bath. To the reaction in the ice-bath was added drop-wise neat acetyl chloride (347 mg, 4.42 mmol, 2.00 eq) and the mixture stirred in the ice-bath for 0.5 h then warmed to 10° C. The reaction mixture was stirred at 10° C. for 2 h. Additional neat acetyl chloride (200 mg) was added and the reaction mixture stirred at 10° C. for 20 additional hours. To the reaction mixture was added water (100 ml), and the mixture was then shaken and separated. The organic phase was washed with brine (100 ml), dried over MgSO₄, filtered and concentrated under reduced pressure to provide 900 mg of title compound as a yellowish oil. The crude product was taken to the next step without further purification.

### Step 3

### Preparation of (2S,4R)-1-(((9H-fluoren-9-yl)methoxy)carbonyl)-4-(acetylamino)pyrrolidine-2-carboxylic Acid

To a 100 ml round bottom flask was added i-PrOH (21 ml) water (9 ml) and anhydrous CaCl₂ (2660 mg, 24 mmol, 10.9 eq.). The reaction was stirred at 10° C. for 10 minutes. To a second 100 ml round bottom flask were added (2S,4R)-1-(9H-fluoren-9-yl)methyl 2-methyl-4-acetamidopyrrolidine-1,2-dicarboxylate (900 mg, 2.20 mmol, 1.0 eq.) followed by the solution of 0.8 M CaCl₂ in i-PrOH/H₂O (27 ml) prepared in the first round bottom flask. To the reaction mixture was then added solid NaOH (123 mg, 3.08 mmol, 1.4 eq.). The suspension was stirred at 10° C. for 46 h. The mixture was adjusted to pH ˜5 with the addition of saturated citric acid, followed by the addition of water (50 ml) and DCM (100 ml). The organic phase was separated and washed with brine (50 ml), dried over MgSO₄, filtered and concentrated under reduced pressure to provide 1.2 g of a crude product as an oil. Purification of the crude product by flash chromatography provided 600 mg of the title compound as an oil. ¹H NMR (400 MHz, CD₃OD) δ 8.37-8.35 (d, 0.6H), 7.82-7.79 (m, 2H), 7.65-7.61 (m, 2H), 7.42-7.38 (m, 2H), 7.34-7.30 (m, 2H), 4.48-4.17 (m, 5H), 3.78-3.73 (m, 1H), 3.42-3.37 (m, 1H), 2.32-2.23 (m, 2H), 1.95-1.94 (d, 3H). HPLC retention time=3.95 min. Column: Ultimate XB-C18, 3 μM, 3.0*50 mm. Mobile Phase: 1.0% MeCN in water (0.1% TFA) to 5% MeCN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 minutes and hold two minutes. Flow rate: 1.2 ml/min.

SFC chiral column retention time=3.98 min. Column: Chiralpak AS-3 150×4.6 mm I.D., 3 um; Mobile phase: A: CO₂ B: ethanol (0.05% DEA); Gradient: from 5% to 40% of B in 5.5 min and hold 40% for 3 min, then 5% of B for 1.5 min; Flow rate: 2.5 ml/min; Column temperature: 40° C.

### Preparation 5

### (2S,4S)-1-(((9H-fluoren-9-yl)methoxy)carbonyl)-4-(acetylamino)pyrrolidine-2-carboxylic Acid

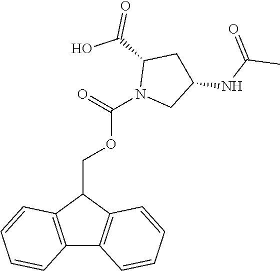 [SMILES: CC(=O)N[C@H]1C[C@@H](C(=O)O)N(C(=O)OCC2c3ccccc3-c3ccccc32)C1]

### Step 1

### Preparation of (2S,4S)-1-(9H-fluoren-9-yl)methyl 2-methyl 4-aminopyrrolidine-1,2-dicarboxylate Hydrochloride

(2S,4S)-1-(((9H-fluoren-9-yl)methoxy)carbonyl)-4-(tert-butoxycarbonylamino)pyrrolidine-2-carboxylic acid (2.00 g, 4.420 mmol, 1.00 eq) was dissolved in MeOH (60 ml) and the reaction mixture placed in an ice-bath. This was followed by the drop-wise addition of neat SOCl₂ (1.05 g, 8.84 mmol, 2.00 eq). After the addition, the mixture was stirred at 70° C. for 1 h. The reaction mixture was cooled to 20° C. and concentrated under reduced pressure to provide 1.7 g of the title compound as a solid, which was used in the next step without purification.

### Step 2

### Preparation of (2S,4S)-1-(9H-fluoren-9-yl)methyl 2-methyl 4-acetamidopyrrolidine-1,2-dicarboxylate

To a solution of (2S,4S)-1-(9H-fluoren-9-yl)methyl 2-methyl 4-aminopyrrolidine-1,2-dicarboxylate hydrochloride (1000 mg, 2.48 mmol, 1.00 eq) in DCM (80 ml) was added Na₂CO₃ (789 mg, 7.45 mmol, 3.00 eq). The reaction mixture was placed in an ice-bath and stirred for 5 min prior to the addition of neat acetyl chloride (390 mg, 4.96 mmol, 2.00 eq). After the addition, the mixture was stirred in the ice-bath for 0.5 h. The reaction was then warmed to 10° C. and stirred for 17 h. To the reaction was added water (100 ml). The organic phase was separated and was washed with brine (100 ml), dried over MgSO₄, filtered and concentrated to provide 1.0 g of the title compound, which was used in the next step without purification.

### Step 3

### Preparation of (2S,4S)-1-(((9H-fluoren-9-yl)methoxy)carbonyl)-4-(acetylamino)pyrrolidine-2-carboxylic Acid

To a first 100 ml round bottom flask was added i-PrOH (21 ml), water (9 ml) and anhydrous CaCl₂ (2660 mg, 24 mmol, 10.9 eq.) and the mixture stirred at 10° C. for 10 minutes. To a second 100 ml round bottom flask was added (2S,4S)-1-(9H-fluoren-9-yl)methyl 2-methyl 4-acetamidopyrrolidine-1,2-dicarboxylate (1010 mg, 2.47 mmol, 1.0 eq.) followed by the 0.8 M CaCl₂ in i-PrOH/H₂O (27 ml) solution prepared in the first round bottom flask. To the reaction mixture was then added solid NaOH (138 mg, 3.46 mmol, 1.4 eq.) and suspension stirred at 10° C. for 22 h. The mixture was adjusted to pH ˜5 with the addition of saturated aqueous citric acid solution and then most of the iPrOH was removed under reduced pressure. To the aqueous residue was added water (50 ml) and DCM (100 ml). The organic phase was separated and washed with brine (50 ml), dried over MgSO₄, filtered and concentrated under reduced pressure to a crude oil. Purification of the crude oil by flash chromatography provided 380 mg of the title compound as a solid. ¹H NMR (400 MHz, CD₃OD) δ 7.81-7.78 (dd, 2H), 7.65-7.60 (m, 2H), 7.41-7.37 (m, 2H), 7.34-7.29 (m, 2H), 4.41-4.16 (m, 5H), 3.87-3.75 (m, 1H), 3.35-3.26 (m, 1H), 2.67-2.54 (m, 1H), 2.07-1.92 (m, 4H). HPLC retention time=4.01 min. Column: Ultimate XB-C18, 3 μM, 3.0*50 mm. Mobile Phase: 1.0% MeCN in water (0.1% TFA) to 5% MeCN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 min, and hold two minutes. Flow rate: 1.2 ml/min. SFC chiral column retention time=3.04 min. Column: Chiralcel OJ-H 150×4.6 mm I.D., 5 μm; Mobile phase: A: supercritical CO₂; B: ethanol (0.05% DEA); Gradient: from 5% to 40% of B in 5.5 min and hold 40% for 3 min, then 5% of B for 1.5 min. Flow rate: 2.5 ml/min; Column temperature: 40° C.

### Preparation 6

### (2S,4S)-1-(((9H-fluoren-9-yl)methoxy)carbonyl)-4-((2-(tert-butoxy)-2-oxoethyl)(tert-butoxycarbonyl)amino)pyrrolidine-2-carboxylic Acid

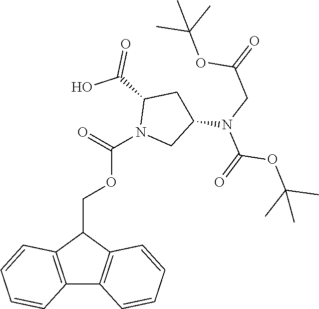 [SMILES: CC(C)(C)OC(=O)CN(C(=O)OC(C)(C)C)[C@H]1C[C@@H](C(=O)O)N(C(=O)OCC2c3ccccc3-c3ccccc32)C1]

### Step 1

### Preparation of (2S,4S)-1-(9H-fluoren-9-yl)methyl 2-methyl 4-aminopyrrolidine-1,2-dicarboxylate Hydrochloride

To a stirred reaction mixture of (2S,4S)-1-(((9H-fluoren-9-yl)methoxy)carbonyl)-4-(tert-butoxycarbonylamino)pyrrolidine-2-carboxylic acid (500 mg, 1.10 mmol, 1.00 eq) in MeOH (15 ml) in an ice-bath was added drop-wise, neat SOCl₂ (263 mg, 2.21 mmol, 2.00 eq). After the addition, the mixture was stirred at 70° C. for 1.5 h. The reaction mixture was cooled to 20° C. and was concentrated under reduced pressure to provide 445 mg of the title compound as a solid, which was used in the next step without purification.

### Step 2

### Preparation of (2S,4S)-1-(9H-fluoren-9-yl)methyl 2-methyl 4-(2-tert-butoxy-2-oxoethylamino)pyrrolidine-1,2-dicarboxylate

To a stirred solution of (2S,4S)-1-(9H-fluoren-9-yl)methyl 2-methyl 4-aminopyrrolidine-1,2-dicarboxylate hydrochloride (295 mg, 0.73 mmol, 1.00 eq) in DMF (20 ml) was added Na₂CO₃ (233 mg, 2.20 mmol, 3.00 eq) and the reaction stirred at 10° C. for 5 min. A solution of tert-butyl bromoacetate (143 mg, 0.732 mmol, 1.00 eq) in DMF (4 ml) was added drop-wise. After the addition, the mixture was stirred at 10° C. for 2 h. To the reaction was added additional tert-butyl bromoacetate (143 mg, 0.732 mmol, 1.00 eq) and the mixture stirred at 10° C. for additional 17 h. The reaction mixture was diluted with EtOAc (120 ml), and was subsequently washed with water (100 ml×2) and brine (100 ml). The organic layer was dried over MgSO₄, filtered and concentrated under reduced pressure to provide 450 mg of an oil. The oil was combined with a second batch (54 mg) of oil (prepared in the manner described above). The combined batch was purified by flash chromatography to provide 240 mg of the title compound as an oil. HPLC retention time=4.16 min. Column: Ultimate XB-C18, 3 μm, 3.0×50 mm. Mobile Phase: 1.0% MeCN in water (0.1% TFA) to 5% ACN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 min, and hold two minutes. Flow rate: 1.2 ml/min. LCMS: m/z 481.1 (M+H⁺)

### Step 3

### Preparation of (2S,4S)-1-(9H-fluoren-9-yl)methyl-2-methyl-4-((2-tert-butoxy-2-oxoethyl)(tert-butoxycarbonyl)amino)pyrrolidine-1,2-dicarboxylate

A solution of (2S,4S)-1-(9H-fluoren-9-yl)methyl-2-methyl-4-(2-tert-butoxy-2-oxoethylamino)pyrrolidine-1,2-dicarboxylate (240 mg, 0.499 mmol, 1.00 eq.) and Na₂CO₃ (159 mg, 1.50 mmol, 3.00 eq.) in dioxane (10 ml) and water (10 ml) was stirred at 10° C. for 10 min then placed in an ice-water bath. To the reaction in the ice-water bath was added neat (Boc)₂O (436 mg, 2.00 mmol, 4.00 eq.). The resulting mixture was stirred in the ice-bath for about 30 min then the ice-water bath was removed. The reaction was stirred at 10° C. for 18 h. To the reaction were added EtOAc (80 ml) and water (80 ml). The organic phase was separated and dried over MgSO₄, filtered and concentrated under reduced pressure to provide 480 mg of the title compound as an oil, which was used in the next step without purification.

### Step 4

### Preparation of (2S,4S)-1-(((9H-fluoren-9-yl)methoxy)carbonyl)-4-((2-tert-butoxy-2-oxoethyl)(tert-butoxycarbonyl)amino)pyrrolidine-2-carboxylic Acid

To a first 100 ml round bottom flask was added i-PrOH (7 ml), water (3 ml) and anhydrous CaCl₂ (888 mg, 8 mmol, 9.68 eq.). The reaction mixture was stirred at 10° C. for 10 minutes. To another 100 ml round bottom flask was added (2S,4S)-1-(9H-fluoren-9-yl)methyl-2-methyl-4-((2-tert-butoxy-2-oxoethyl)(tert-butoxycarbonyl)amino)pyrrolidine-1,2-dicarboxylate (480 mg, 0.83 mmol, 1.0 eq.) followed by 5 ml of the aqueous mixture of ˜0.8 M CaCl₂ in i-PrOH/water prepared in the first round bottom flask, and then NaOH (46.3 mg, 1.16 mmol, 1.4 eq.). The resulting reaction mixture was stirred at 10° C. for 17 h. To the reaction mixture was added additional NaOH (50 mg) and the mixture stirred at 10° C. for additional 15 h. The reaction mixture was adjusted to pH˜4 by the addition of aqueous saturated citric acid solution followed by the addition of water (30 ml). The reaction was extracted with EtOAc (30 ml×2) and the combined organic phase dried over MgSO₄, filtered and concentrated to provide 200 mg of a yellow oil. Purification of the crude oil via flash chromatography provided 200 mg of a solid. Further purification of the solid by preparative HPLC (Column: Agela Durashell C18 150×25 mm, 5 μm; Eluent: 60% acetonitrile in water; Gradient: 60 to 80% acetonitrile in water over 11 minutes; Flow rate: 35 ml/min) provided 75 mg of the title compound as a solid. HPLC retention time=5.18 min. Column: Ultimate XB-C18, 3 μm, 3.0×50 mm. Mobile Phase: 1.0% MeCN in water (0.1% TFA) to 5% MeCN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 min, and hold two minutes. Flow rate: 1.2 ml/min. SFC chiral column retention time=3.76 min. Column: Chiralcel OD-3 100×4.6 mm I.D., 3 um; Mobile phase: A: supercritical CO₂; B: ethanol (0.05% DEA); Gradient: from 5% to 40% of B in 4.5 min and hold 40% for 2.5 min, then 5% of B for 1 min. Flow rate: 2.8 ml/min; Column temperature: 40° C.

### Preparation 7

### (2S,4R)-1-(((9H-fluoren-9-yl)methoxy)carbonyl)-4-((2-tert-butoxy-2-oxoethyl)(tert-butoxycarbonyl)amino)pyrrolidine-2-carboxylic Acid

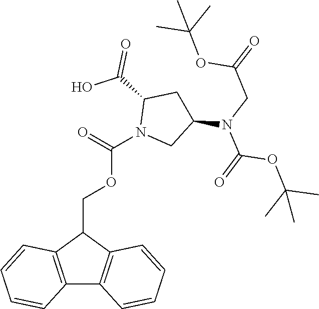 [SMILES: CC(C)(C)OC(=O)CN(C(=O)OC(C)(C)C)[C@@H]1C[C@@H](C(=O)O)N(C(=O)OCC2c3ccccc3-c3ccccc32)C1]

### Step 1

### Preparation of (2S,4R)-1-(9H-fluoren-9-yl)methyl 2-methyl 4-aminopyrrolidine-1,2-dicarboxylate Hydrochloride

(2S,4R)-1-(((9H-fluoren-9-yl)methoxy)carbonyl)-4-(tert-butoxycarbonylamino)pyrrolidine-2-carboxylic acid (500 mg, 1.10 mmol, 1.00 eq) was dissolved in MeOH (20 ml) and placed in an ice-water bath. To the reaction mixture was added drop-wise neat SOCl₂ (263 mg, 2.21 mmol, 2.00 eq) and the mixture stirred at 70° C. for 1.5 h. The reaction was cooled to 30° C. and was concentrated under reduced pressure to provide 445 mg of the title compound as a solid, which was used in the next step without purification.

### Step 2

### Preparation of (2S,4R)-1-(9H-fluoren-9-yl)methyl 2-methyl 4-(2-tert-butoxy-2-oxoethylamino)pyrrolidine-1,2-dicarboxylate

To a solution of (2S,4R)-1-(9H-fluoren-9-yl)methyl 2-methyl 4-aminopyrrolidine-1,2-dicarboxylate hydrochloride (445 mg, 1.10 mmol, 1.00 eq) in DMF (30 ml) was added Na₂CO₃ (351 mg, 3.31 mmol, 3.00 eq). The suspension was stirred at 10° C. for 5 min. A solution of tert-butyl bromoacetate (215 mg, 1.10 mmol, 1.00 eq) in DMF (4 ml) was added drop-wise. After the addition, the mixture was stirred at 10° C. for 5 h. Additional neat tert-butyl bromoacetate (215 mg, 1.10 mmol, 1.00 eq) was added and the mixture was stirred at 10° C. for additional 17 h. The mixture was diluted with EtOAc (180 ml), washed with water (180 ml×2) and brine (100 ml). The organic phase was dried over MgSO₄, filtered and concentrated under reduced pressure to provide a colorless oil. The oil was purified by flash chromatography to provide 305 mg of the title compound as an oil.

### Step 3

### Preparation of (2S,4R)-1-(9H-fluoren-9-yl)methyl-2-methyl-4-((2-tert-butoxy-2-oxoethyl)(tert-butoxycarbonyl)amino)pyrrolidine-1,2-dicarboxylate

(2S,4R)-1-(9H-fluoren-9-yl)methyl-2-methyl-4-(2-tert-butoxy-2-oxoethylamino)pyrrolidine-1,2-dicarboxylate (305 mg, 0.635 mmol, 1.00 eq.) and Na₂CO₃ (202 mg, 1.90 mmol, 3.00 eq.) were dissolved in a mixture of dioxane (15 ml) and water (15 ml). The mixture was stirred at 10° C. for 10 min then placed in an ice-water bath. To the reaction in the ice-water bath was added neat (Boc)₂O (554 mg, 2.54 mmol, 4.00 eq.). The resulting mixture was stirred in the ice-water bath for 30 min then the ice-water bath was removed. The mixture was warmed to 10° C. and stirred for 18 h. To the reaction mixture was added EtOAc (100 ml) and H₂O (100 ml). The organic phase was separated and dried over MgSO₄, filtered and concentrated under reduced pressure to provide 730 mg of the title compound as an oil, which was used in the next step without purification.

### Step 4

### Preparation of (2S,4R)-4-((2-tert-butoxy-2-oxoethyl)(tert-butoxycarbonyl)amino)pyrrolidine-2-carboxylic Acid

To a 100 ml round bottom flask was added i-PrOH (14 ml), water (6 ml) and anhydrous CaCl₂ (1780 mg, 16 mmol). The reaction was stirred at 10° C. for 10 minutes. To a second 100 ml round bottom flask was added (2S,4R)-1-(9H-fluoren-9-yl)methyl-2-methyl-4-((2-tert-butoxy-2-oxoethyl)(tert-butoxycarbonyl)amino)pyrrolidine-1,2-dicarboxylate (730 mg, 1.26 mmol, 1.0 eq.), followed by 10 ml of the ˜0.8 M CaCl₂ solution in i-PrOH/water from the first round bottom flask, and then solid NaOH (70.4 mg, 1.76 mmol, 1.4 eq.). The reaction mixture was stirred at 10° C. for 41 h. To the reaction mixture was added more NaOH (80 mg) and the mixture was stirred for additional 72 h. The reaction mixture was heated to 50° C. and stirred at that temperature for 24 h. To the reaction was added additional NaOH (100 mg) and the mixture was stirred at 50° C. for an additional 6 h. The reaction mixture was adjusted to pH ˜4 with the addition of aqueous saturated citric acid, followed by the addition of water (10 ml). The mixture was extracted with EtOAc (30 ml×2) and the combined organic phase dried over MgSO₄, filtered and concentrated under reduced pressure to provide 150 mg of the title compound as an oil, which was used in the next step without purification.

### Step 5

### Preparation of (2S,4R)-1-(((9H-fluoren-9-yl)methoxy)carbonyl)-4-((2-tert-butoxy-2-oxoethyl)(tert-butoxycarbonyl)amino)pyrrolidine-2-carboxylic Acid

A suspension of (2S,4R)-4-((2-tert-butoxy-2-oxoethyl)(tert-butoxycarbonyl)amino)pyrrolidine-2-carboxylic acid (150 mg, 0.436 mmol, 1.00 eq.) and Na₂CO₃ (200 mg, 1.89 mmol, 4.33 eq.) in dioxane (20 ml) was stirred at 10° C. for 10 min, then placed in an ice-water bath. To the reaction in an ice-water bath was added in portions neat Fmoc-OSu (250 mg, 0.741 mmol, 1.70 eq.). The resulting mixture was stirred in the ice-water bath for 30 min then the ice-water bath was removed. The mixture was warmed to 10° C. and stirred for 17 h. The mixture was adjusted to pH ˜4 by the addition of aqueous saturated citric acid, followed by the addition of EtOAc (50 ml) and water (50 ml). The organic phase was separated and was dried over MgSO₄, filtered and concentrated under reduced pressure to provide 450 mg of an oil. Purification of the oil by preparative HPLC (Column: Agela Durashell C18 150×25 mm, 5 μm; Eluent: 50% acetonitrile in water; Gradient: 50 to 80% acetonitrile in water over 12 minutes; Flow rate: 25 ml/min) provided 140 mg of the title compound as a solid. LCMS: MS=589.1 (M+Na). HPLC retention time=5.41 min. Column: Ultimate XB-C18, 3 μm, 3.0×50 mm. Mobile Phase: 1.0% MeCN in water (0.1% TFA) to 5% MeCN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 min, and hold two minutes. Flow rate: 1.2 ml/min. SFC chiral column retention time=3.48 min. Column: Chiralcel OD-3 100×4.6 mm I.D., 3 μm; Mobile phase: A: supercritical CO₂; B: ethanol (0.05% DEA); Gradient: from 5% to 40% of B in 4.5 min and hold 40% for 2.5 min, then 5% of B for 1 min. Flow rate: 2.8 ml/min; Column temperature: 40° C.

### Preparation 8

### (S)-2-((2-(tert-butoxy)-2-oxoethyl)(tert-butoxycarbonyl)amino)-3-(3,5-dibromophenyl)propanoic Acid

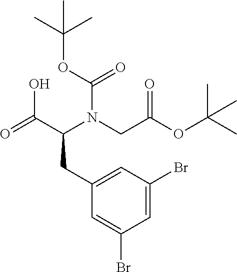 [SMILES: CC(C)(C)OC(=O)CN(C(=O)OC(C)(C)C)[C@@H](Cc1cc(Br)cc(Br)c1)C(=O)O]

### Step 1

### Preparation of methyl 2-(tert-butoxycarbonylamino)-3-(3,5-dibromophenyl)acrylate

5,5-dibromobenzaldehyde (15.0 g, 56.837 mmol, 1.0 eq.) was dissolved in DCM (50 ml) and the reaction degassed by vacuum and purged with N₂. To the mixture was slowly added DBU (10.4 g, 68.2 mmol, 1.2 eq.) and the reaction was placed in an ice-water bath. In a second flask N-(tert-butoxycarbonyl)-2-phosphonoglycine trimethyl ester (16.9 g, 56.8 mmol, 1.0 eq.) was dissolved in DCM (50 ml) and this mixture was added drop-wise via a syringe over a period of 1 h to the reaction mixture in the ice-water bath. After the addition, the mixture was stirred in the ice-water bath for 30 minutes. The ice-water bath was removed and the mixture warmed to about 20° C. with stirring for 16 hours. The reaction mixture was diluted with water (500 ml) and acidified to pH ˜4 with the addition of citric acid (20% w.t). The organic layer was separated, dried over Na₂SO₄, filtered and concentrated under reduced pressure to provide 30 g of a solid. Purification of the solid material by flash chromatography provided 7.6 g of the title compound as a solid. ¹H NMR (400 MHz, CDCl₃) δ 7.58-7.55 (m, 3H), 7.10 (s, 1H), 6.50 (br, s, 1H), 3.87 (s, 3H), 1.42 (br, s, 9H). HPLC retention time=5.23 min. Column: Ultimate XB-C18, 3 μm, 3.0×50 mm. Mobile Phase: 1.0% MeCN in water (0.1% TFA) to 5% MeCN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 min, and hold two minutes. Flow rate: 1.2 ml/min. LCMS: MS=457.9 (M+Na)

### Step 2

### Preparation of (S)-methyl 2-(tert-butoxycarbonylamino)-3-(3,5-dibromophenyl)propanoate

To a 250 ml Parr bottle were added methyl 2-(tert-butoxycarbonylamino)-3-(3,5-dibromophenyl)acrylate (6.0 g, 13.79 mmol, 1.0 eq.) and (−)-1,2-bis[(2R,5R)-2,5-diethylphospholano]benzene(1,5-cyclooctadiene)rhodium(I) trifluoromethanesulfonate (100 mg) followed by MeOH (100 ml). The reaction mixture was degassed by vacuum and purged with N₂ 10 times. The reaction was then back filled with H₂ gas, degassed by vacuum, purged and refilled 3 times with H₂ gas. The reaction was then filled with H₂ gas to a pressure of 50 psi and heated with stirring to 50° C. for 24 hrs. After cooling to room temperature, the reaction mixture was filtered and concentrated to provide 6.0 g of the title compound as an oil, which was taken to the next step without further purification. ¹H NMR (400 MHz, CDCl₃) δ 7.56-7.55 (t, 3H), 7.22 (bs, 2H), 5.05-5.03 (d, 1H), 4.55-4.53 (dd, 1H), 3.74 (s, 3H), 3.13-3.08 (m, 1H), 2.99-2.94 (m, 1H), 1.44 (s, 9H).

### Step 3

### Preparation of (S)-methyl 2-amino-3-(3,5-dibromophenyl)propanoate Hydrochloride

(S)-methyl 2-(tert-butoxycarbonylamino)-3-(3,5-dibromophenyl)propanoate (4.73 g, 10.8 mmol, 1.0 eq.) was dissolved in EtOAc (20 ml) and 25 ml of a 4 M solution of HCl in EtOAc was added. The resulting solution was stirred at 15° C. for 3 hours. The reaction mixture was concentrated under reduced pressure to provide 4.0 g of the title compound as a solid, which was used in the next step without purification. ¹H NMR (400 MHz, DMSO-d₆) δ 8.68 (bs, 3H), 7.77-7.76 (t, 1H), 7.55-7.54 (d, 2H), 4.39-4.36 (t, 1H), 3.71 (s, 3H), 3.17-3.15 (d, 2H)

### Step 4

### Preparation of (S)-methyl 2-(2-tert-butoxy-2-oxoethylamino)-3-(3,5-dibromophenyl)propanoate

(S)-methyl 2-amino-3-(3,5-dibromophenyl)propanoate hydrochloride (4.0 g, 11.0 mmol, 1.0 eq.) was dissolved in DMF (60 ml) and DIPEA (8.76 ml, 42.8 mmol, 4.0 eq.) was added followed by the drop-wise addition of neat tert-butyl bromoacetate (2.51 g, 12.9 mmol, 1.2 eq.). The resulting solution was stirred at 15° C. for 16 hours. Additional tert-butyl bromoacetate (2.09 g, 10.7 mmol, 1.0 eq.) was added drop-wise to the reaction mixture. The resulting solution was stirred at 15° C. for additional 24 hours. To the mixture was added water (150 ml) and the solution was extracted with EtOAc (100 ml×3). The combined organic layer was washed with brine (200 ml×2), dried over Na₂SO₄, filtered and concentrated under reduced pressure. Purification of the resulting crude residue by flash chromatography provided 4.0 g of the title compound as an oil. ¹H NMR (400 MHz, CDCl₃) δ 7.54-7.53 (t, 1H), 7.30-7.29 (d, 2H), 3.68 (s, 3H), 3.54-3.50 (t, 1H), 3.32-3.22 (q, 2H), 2.96-2.86 (m, 2H), 1.45 (s, 9H)

### Step 5

### Preparation of (S)-methyl 2-((2-tert-butoxy-2-oxoethyl)(tert-butoxycarbonyl)amino)-3-(3,5-dibromophenyl)propanoate

(S)-methyl 2-(2-tert-butoxy-2-oxoethylamino)-3-(3,5-dibromophenyl)propanoate (3.0 g, 6.65 mmol, 1.0 eq.) was dissolved in DCM (50 ml) and (Boc)₂O (4.35 g, 19.9 mmol, 3.0 eq.) was added. The mixture was heated to 40° C. followed by the addition of neat DMAP (1.62 g, 13.3 mmol, 2.0 eq.). Additional (Boc)₂O (39.2 g, 40.0 mmol, 27.0 eq.) was added to the reaction and the mixture stirred at 40° C. for 2 hours. Purification of the crude mixture by flash chromatography provided 3.2 g of the title compound as an oil.

### Step 6

### Preparation of (S)-2-((2-tert-butoxy-2-oxoethyl)(tert-butoxycarbonyl)amino)-3-(3,5-dibromophenyl)propanoic Acid

(S)-methyl 2-((2-tert-butoxy-2-oxoethyl)(tert-butoxycarbonyl)amino)-3-(3,5-dibromophenyl)propanoate (3.2 g, 5.81 mmol, 1.0 eq.) was dissolved in a mixture of THF (30 ml) and water (30 ml). To the reaction was added neat LiOH—H₂O (536 mg, 12.8 mmol, 2.2 eq.) and the resulting mixture was stirred at 15° C. for 40 minutes. The reaction was acidified to pH ˜4 with the addition of aqueous citric acid (5% wt) and was concentrated under reduced pressure to remove most of the organic solvent. The resulting residue was diluted with water (100 ml) and was extracted with EtOAc (100 ml×2). The combined organic extract was dried over Na₂SO₄, filtered and concentrated under reduced pressure to provide 3.4 g of an oil. The oil was combined with 1.2 g of a second batch of oil prepared according to the above protocol. Purification of the combined batches by flash chromatography provided 2.94 g of the title compound as a solid. ¹H NMR (400 MHz, DMSO-d₆) δ 12.84 (bs, 1H), 7.67-7.64 (m, 1H), 7.51-7.47 (m, 2H), 4.79-4.75 (m, 0.6H), 4.65-4.61 (m, 0.5H), 3.84-3.70 (m, 2H), 3.19-3.11 (m, 1H), 3.04-2.97 (m, 1H), 1.37-1.29 (m, 18H). HPLC retention time=5.50 min. Column: Ultimate XB-C18, 3 μm, 3.0×50 mm. Mobile Phase: 1.0% MeCN in water (0.1% TFA) to 5% MeCN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 min, and hold two minutes. Flow rate: 1.2 ml/min. Optical rotation: −49.518 in MeOH, c=1.4 g/100 ml

### Preparation 9

### (2S,4R)-1-(((9H-fluoren-9-yl)methoxy)carbonyl)-4-(2-tert-butoxy-2-oxoethoxy)pyrolidine-2-carboxylic Acid

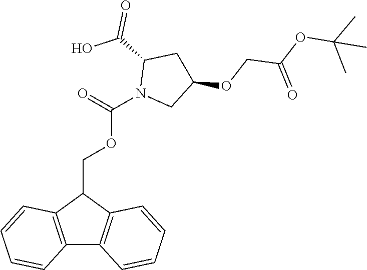 [SMILES: CC(C)(C)OC(=O)CO[C@@H]1C[C@@H](C(=O)O)N(C(=O)OCC2c3ccccc3-c3ccccc32)C1]

### Step 1

### Preparation of (2S,4R)-1-(benzyloxycarbonyl)-4-hydroxypyrrolidine-2-carboxylic Acid

To a 3 L flask were added (2S,4R)-4-hydroxypyrrolidine-2-carboxylic acid (87.0 g, 663.46 mmol, 1.0 eq.), water (1500 ml), Na₂CO₃ (141.0 g, 1330 mmol, 2.0 eq.) and NaHCO₃ (55.7 g, 663 mmol, 1.0 eq.). Acetone (250 ml) was added to the solution which was then cooled in an ice-water bath. To the mixture was slowly added Cbz-Cl (141.0 g, 829 mmol, 1.25 eq.). After addition, the reaction mixture was warmed gradually to 22° C. and was stirred for 15 h. The reaction mixture was washed with MTBE (600 ml×2). To the aqueous phase was slowly added aqueous 1 N HCl until pH ˜2 was achieved. The resulting mixture was extracted with EtOAc (1 L×3) and the combined organic layer was dried over MgSO4, filtered and concentrated under reduced pressure to provide 190 g of the title compound as an oil, which was taken to the next step without further purification. LCMS: MS=287.8 (M+H).

### Step 2

### Preparation of (2S,4R)-dibenzyl 4-hydroxypyrrolidine-1,2-dicarboxylate

To a 3 L round bottom flask were added (2S,4R)-1-(benzyloxycarbonyl)-4-hydroxypyrrolidine-2-carboxylic acid (190.0 g, 716 mmol, 1.0 eq.), DMF (2.0 L) and Cs₂CO₃ (117 g, 358 mmol, 0.5 eq.). The resulting mixture was cooled in an ice-water bath and benzyl bromide (172 g, 1000 mmol, 1.4 eq.) was slowly added. After the addition, the resulting suspension was stirred at 22° C. for 5 h. The reaction mixture was diluted with water (5 L) and EtOAc (5 L). The aqueous layer was removed and the organic layer washed with brine (2×3 L), dried over MgSO₄, filtered and concentrated under reduced pressure to provide 250 g of the title compound as an oil, which was used in the next step without purification. LCMS: MS=377.9 (M+23).

### Step 3

### Preparation of (2S,4R)-dibenzyl 4-(2-tert-butoxy-2-oxoethoxy)pyrrolidine-1,2-dicarboxylate

To a 3 L round bottom flask under a nitrogen gas atmosphere was added dry THF (1 L) and NaH (14.3 g, 60% w.t. in mineral oil, 359 mmol, 1.5 eq.). To the reaction mixture was added tetrabutylammonium iodide (8.83 g, 23.9 mmol, 0.1 eq.) followed by tert-butyl bromoacetate (187 g, 957 mmol, 4.0 eq.). The resultant suspension was stirred at 22° C. for 0.5 h. A solution of (2S,4R)-dibenzyl 4-hydroxypyrrolidine-1,2-dicarboxylate (85.0 g, 239 mmol, 1.0 eq.) in THF (300 ml) was added drop-wise. After the addition, the suspension was stirred at 22° C. for 16 h. To the reaction was added a solution of saturated aqueous NH₄Cl (100 ml). The organic layer was separated and the aqueous layer was extracted with EtOAc (150 ml×3). The combined organic layers were dried over MgSO₄, filtered and concentrated under reduced pressure to provide 160 g of a yellow oil. Purification of the oil by flash chromatography provided 60.0 g of the title compound as a clear oil. LCMS: MS: 492.2 (M+Na).

### Step 4

### Preparation of (2S,4R)-4-(2-tert-butoxy-2-oxoethoxy)pyrrolidine-2-carboxylic Acid

To a 2 L hydrogenation vessel equipped with a magnetic stirrer were added (2S,4R)-dibenzyl 4-(2-tert-butoxy-2-oxoethoxy)pyrrolidine-1,2-dicarboxylate (40.0 g, 85.192 mmol, 1.0 eq.), ethanol (500 ml), EtOAc (500 ml) and Pd/C (5.44 g, 10% on wet carbon). The resulting black suspension was evacuated under vacuum and refilled with H₂ (3×). The vessel was pressurized to 50 psi of hydrogen atmosphere and was stirred at 22° C. for 14 h. The black suspension was filtered through a celite pad and the filtrate was concentrated under reduced pressure to provide 20 g of a white solid, which was taken to the next step with no further purification. ¹H NMR (400 MHz, CD₃OD) δ 4.38-4.36 (m, 1H), 4.22-4.17 (m, 1H), 4.13-4.04 (m, 2H), 3.50-3.39 (m, 2H), 2.61-2.56 (m, 1H), 2.11-2.03 (m, 1H), 1.49 (s, 9H)

### Step 5

### Preparation of (2S,4R)-1-(((9H-fluoren-9-yl)methoxy)carbonyl)-4-(2-tert-butoxy-2-oxoethoxy)pyrrolidine-2-carboxylic Acid

To a 1 L round bottom flask were added (2S,4R)-4-(2-tert-butoxy-2-oxoethoxy)pyrrolidine-2-carboxylic acid (15 g, 61.16 mmol, 1.0 eq.), DCM (500 ml) and DIPEA (23.7 g, 183 mmol, 3.00 eq.) and the mixture was cooled in an ice-water bath. To the reaction in the ice-water bath was then added Fmoc-OSu (20.6 g, 61.2 mmol, 1.0 eq.) and the resultant mixture was stirred in the ice-water bath for 30 minutes. The ice-water bath was removed and the mixture warmed to 22° C. and kept at that temperature for 4 h. Water (200 ml) was added and the reaction was then extracted with EtOAc (300 ml×3). The combined organic extracts were concentrated under reduced pressure to provide 55 g of a yellow oil. Purification of the oil by preparative HPLC (Column: Phenomenex Synergi Max-RP 250×50 mm×10 μm; Mobile phase: from 40% MeCN (0.225% FA) in water to 70% MeCN (0.225% FA) in water; Flow rate: 30 ml/min; Wavelength: 220 nm) provided 23 g of the title compound as a solid. ¹H NMR (400 MHz, CDCl₃) δ 7.77-7.75 (d, 1.2H), 7.71-7.69 (d, 0.8H), 7.59-7.51 (m, 2H), 7.42-7.25 (m, 4H), 4.56-4.52 (t, 0.68H), 4.47-4.32 (m, 2.4H), 4.28-4.11 (m, 2H), 4.01-3.92 (m, 2H), 3.79-3.58 (m, 2H), 2.55-2.50 (m, 0.39H), 2.45-2.39 (m, 0.65H), 2.32-2.26 (m, 0.65H), 2.19-2.12 (m, 0.38H), 1.49-1.47 (d, 9H). HPLC retention time=4.66 min. Column: Ultimate XB-C18, 3 μM, 3.0*50 mm. Mobile Phase: 1.0% MeCN in water (0.1% TFA) to 5% MeCN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 min, and hold two minutes. Flow rate: 1.2 ml/min. SFC chiral column retention time=3.18 min. Column: Chiralpak AD-3 100×4.6 mm I.D., 3 um; Mobile phase: A: CO₂ B: ethanol (0.05% DEA); Gradient: from 5% to 40% of B in 4.5 min and hold 40% for 2.5 min, then 5% of B for 1 min. Flow rate: 2.8 ml/min; Column temperature: 40° C.

### Preparation 10

### (S)-2-((((9H-fluoren-9-yl)methoxy)carbonyl)amino)-3-(1-(tert-butoxycarbonyl)-1H-indazol-3-yl)propanoic Acid

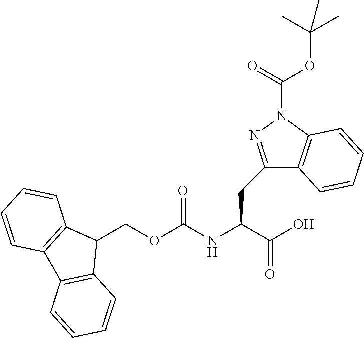 [SMILES: CC(C)(C)OC(=O)n1nc(C[C@H](NC(=O)OCC2c3ccccc3-c3ccccc32)C(=O)O)c2ccccc21]

### Step 1

### Preparation of methyl 2-(((benzyloxy)carbonyl)amino)-3-(6-bromo-1-(tetrahydro-2H-pyran-2-yl)-1H-indazol-3-yl)acrylate

In a 100 mL round bottom flask (±)-methyl 2-benzyloxycarbonylamino-2-(dimethoxyphosphinyl) acetate (1.2 g, 3.6 mmol) was dissolved in DCM (15 mL). The reaction mixture was carefully degassed under vacuum and purged with N₂ gas. To the reaction was slowly added DBU (591 mg, 3.9 mmol) and the mixture was cooled to 5° C. using an ice-water bath. In a second flask 6-bromo-1-(tetrahydro-2H-pyran-2-yl)-1H-indazole-3-carbaldehyde (1.0 g, 3.2 mmol) was dissolved in DCM (15 mL) and this solution was then added drop wise via a syringe to the reaction mixture at 5° C. After the addition, the mixture was stirred at 5° C. for 30 minutes. The ice-water bath was removed and the mixture allowed to warm to room temperature (˜25° C.) and stirred for a further 16 h. The mixture was concentrated under reduced pressure and the crude product purified via flash chromatography on a reversed-phase column using a solvent mixture of MeCN/H₂O (with a gradient from 0/100% to 100/0%). Pure fractions were collected containing the desired product and these were concentrated under reduced pressure to provide 1.18 g of the title compound as a solid. LCMS: MS=515.9 (M+H)

The above reaction was repeated with the following amounts of starting material and reagents: (±)-methyl 2-benzyloxycarbonylamino-2-(dimethoxyphosphinyl) acetate (3.5 g, 10.7 mmol, 1.1 eq.), DCM (100 mL) DBU (1.8 g, 11.7 mmol) and 6-bromo-1-(tetrahydro-2H-pyran-2-yl)-1H-indazole-3-carbaldehyde (6517-1) (3.0 g, 9.7 mmol). After purification, 0.94 g of the title compound was obtained as a solid. The two batches (1.18 g+0.94 g) were combined to provide a total of 2.12 g of the title compound.

### Step 2

### Preparation of methyl (2S)-2-(((benzyloxy)carbonyl)amino)-3-(6-bromo-1-(tetrahydro-2H-pyran-2-yl)-1H-indazol-3-yl)propanoate

To a 250 mL parr bottle, was added methyl 2-(((benzyloxy)carbonyl)amino)-3-(6-bromo-1-(tetrahydro-2H-pyran-2-yl)-1H-indazol-3-yl)acrylate (2.12 g, 4.12 mmol) and (+)-1,2-Bis((2S,5S)-2,5-diethylphospholano)benzene(1,5-cyclooctadiene)rhodium(1) tetrafluoroborate (150 mg, 0.23 mmol), followed by a mixture of solvents MeOH (50 mL) and THF (50 mL). The solvents were treated just prior to use by passing N₂ gas via a syringe through the solvents for 20 minutes. The resulting reaction mixture was then degassed by vacuum and purged with N₂ gas 10 times. The container was then back filled with H₂ and then degassed, purged and refilled 3 times. The container was then pressurized with H₂ gas to 50 psi and stirred at 50° C. for 22 h. The mixture was cooled to 30° C., then concentrated under reduced pressure to provide 2.13 g of the crude title compound as an oil. LCMS: MS=538.1 (M+Na). HPLC retention time=5.26 min. Column: Ultimate XB-C18, 3 μm, 3.0*50 mm. Mobile Phase: Gradient 1.0% MeCN in water (0.1% TFA) to 5% MeCN in water (0.1% TFA) in 1 min; then from 5% MeCN in water (0.1% TFA) to 100% MeCN (0.1% TFA) in 5 mins; hold at 100% MeCN (0.1% TFA) for 2 minutes then return back to 1.0% MeCN in water (0.1% TFA) at 8.01 min and hold two minutes. Flow rate: 1.2 mL/min.

### Step 3

### Preparation of methyl (S)-2-(((benzyloxy)carbonyl)amino)-3-(6-bromo-1H-indazol-3-yl)propanoate

In a round bottom flask crude methyl (2S)-2-(((benzyloxy)carbonyl)amino)-3-(6-bromo-1-(tetrahydro-2H-pyran-2-yl)-1H-indazol-3-yl)propanoate (2.13 g, 4.13 mmol) was dissolved in MeOH (50 mL) and a solution of HCl (4.0 M in MeOH, 50 mL) was added. The resulting reaction mixture was stirred at 25° C. for 2 h. The mixture was then concentrated under reduced pressure to provide 1.9 g of the crude title compound as a gum. LCMS: MS=432.1 (M+1). HPLC retention time=4.60 min. Column: Ultimate XB-C18, 3 μm, 3.0*50 mm. Mobile Phase: Gradient from 1.0% MeCN in water (0.1% TFA) to 5% MeCN in water (0.1% TFA) in 1 min; then from 5% MeCN in water (0.1% TFA) to 100% MeCN (0.1% TFA) in 5 mins; hold at 100% MeCN (0.1% TFA) for 2 minutes; back to 1.0% MeCN in water (0.1% TFA) at 8.01 min, and hold two minutes. Flow rate: 1.2 mL/min.

### Step 4

### Preparation of tert-butyl (S)-3-(2-(((benzyloxy)carbonyl)amino)-3-methoxy-3-oxopropyl)-6-bromo-1H-indazole-1-carboxylate

To a round bottom flask containing a solution of crude methyl (S)-2-(((benzyloxy)carbonyl)amino)-3-(6-bromo-1H-indazol-3-yl)propanoate (1.9 g, 4.4 mmol) in DCM (100 mL) was added DMAP (1.01 g, 8.2 mmol) and (Boc)₂O (1.80 g, 8.2 mmol). The resulting reaction mixture was stirred at 25° C. for 18 h. Water (100 mL) was added to the reaction mixture followed by slow addition of an aqueous solution of 10% citric acid until pH ˜4 was achieved. The organic phase was then separated and washed with brine (100 mL), dried over MgSO₄, filtered and concentrated under reduced pressure to provide a crude oil. The crude oil was purified by flash chromatography on a silica-gel column (40 g) using a combination of EtOAc and petroleum ether (gradient from 0/100% to 25/75%). The fractions containing the desired product were collected and concentrated to provide a solid that was further purified using reverse phase preparative scale HPLC. (Column: Phenomenex Gemini C18 250*50 10 u; Mobile phase: from 50% MeCN in water (with 0.05% ammonium hydroxide) to 75% MeCN in water (with 0.05% ammonium hydroxide); Flow rate: 120 ml/min; Wavelength: 220 nm). The fractions containing the desired product were concentrated under reduced pressure to remove most of the MeCN and then lyophilized to provide 1.26 g of the title compound as a solid. ¹H NMR (400 MHz, CDCl₃) δ 8.33 (s, 1H), 7.47 (d, 8.0 Hz, 1H), 7.38-7.30 (m, 6H), 5.80 (d, 8.0 Hz, 1H), 5.14-5.06 (m, 2H), 4.86-4.81 (m, 1H), 3.72 (s, 3H), 3.57-3.46 (m, 2H), 1.69 (s, 9H). HPLC retention time=5.25 min. Column: Ultimate XB-C18, 3 μm, 3.0*50 mm. Mobile Phase: 1.0% MeCN in water (0.1% TFA) to 5% MeCN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 min, and hold two minutes. Flow rate: 1.2 mL/min. SFC chiral column retention time=2.88 min. Column: Chiralpak AS-3 150×4.6 mm I.D., 3 um; Mobile phase: A: CO₂ B: ethanol (0.05% DEA); Gradient: from 5% to 40% of B in 5.5 min and hold 40% for 3 min, then 5% of B for 1.5 min. Flow rate: 2.5 mL/min; Column temperature: 40° C.

### Step 5

### Preparation of tert-butyl (S)-3-(2-amino-3-methoxy-3-oxopropyl)-1H-indazole-1-carboxylate

To a 250 mL parr bottle was added a solution of tert-butyl (S)-3-(2-(((benzyloxy)carbonyl)amino)-3-methoxy-3-oxopropyl)-6-bromo-1H-indazole-1-carboxylate (950 mg, 1.78 mmol) in triethylamine (1 mL) and MeOH (20 mL). To the solution was then added dry Pd/C (570 mg, 0.54 mmol) and the resulting suspension was degassed under vacuum and back filled with N₂ gas 3 times. It was then degassed under vacuum and back filled with H₂ gas 3 times. The resulting mixture was degassed under vacuum and back filled with H₂ gas until a pressure of 20 psi was reached and the reaction mixture then stirred at 25° C. for 5 h. The mixture was passed through Kieselguhr filter and the filtrate concentrated under reduced pressure to provide 570 mg of the crude title compound as a gum. LCMS: MS=263.1 (M−56+1)

### Step 6

### Preparation of tert-butyl (S)-3-(2-((((9H-fluoren-9-yl)methoxy)carbonyl)amino)-3-methoxy-3-oxopropyl)-1H-indazole-1-carboxylate

To a 250 mL round bottom flask was added a solution of crude tert-butyl (S)-3-(2-amino-3-methoxy-3-oxopropyl)-1H-indazole-1-carboxylate (570 mg, 1.8 mmol) in DCM (15 mL) and triethylamine (570 mg, 5.64 mmol). The solution was then cooled to 0° C. using an ice-water bath. To the reaction was added Fmoc-OSu (634 mg, 1.88 mmol) in portions (within 5 minutes) and the mixture was then stirred in the ice-bath for 30 minutes. The ice-water bath was removed and the mixture allowed to warm to room temperature (25° C.) and then stirred at that temperature for about 3 h. The reaction mixture was then concentrated under reduced pressure to provide 1.1 g of a crude gum. Purification of the crude gum was carried out by flash chromatography on a C-18 column (120 g) employing a MeCN/H₂O solvent mixture (gradient from 0-70%). The fractions with desired product were combined and concentrated to provide 550 mg of the title compound as a solid. LCMS: MS=564.1 (M+23)

### Step 7

### Preparation of (S)-2-((((9H-fluoren-9-yl)methoxy)carbonyl)amino)-3-(1-(tert-butoxycarbonyl)-1H-indazol-3-yl)propanoic Acid

To a 100 mL round bottom flask was added i-PrOH (10 mL), H₂O (10 mL) and anhydrous CaCl₂ (1.6 g, 14.8 mmol). The mixture was stirred at room temperature (28° C.) for 10 minutes. To the mixture was then added tert-butyl (S)-3-(2-((((9H-fluoren-9-yl)methoxy)carbonyl)amino)-3-methoxy-3-oxopropyl)-1H-indazole-1-carboxylate (400 mg, 0.74 mmol, 1.0 eq.) followed by NaOH (89 mg, 2.2 mmol). The resulting reaction mixture was stirred at room temperature (28° C.) for 2 h. To the reaction was then added formic acid until a pH ˜6 was reached. The crude reaction mixture was purified by reverse phase HPLC on a C-18 column (120 g), eluting with a MeCN/H₂O solvent mixture (gradient from 0/100% to 80/20%). The fractions containing the desired product were combined and concentrated by lyophilization to provide 350 mg of the title compound as a solid. The reaction was repeated with the following amounts of starting material and reagents: tert-butyl (S)-3-(2-((((9H-fluoren-9-yl)methoxy)carbonyl)amino)-3-methoxy-3-oxopropyl)-1H-indazole-1-carboxylate (200 mg, 0.37 mmol), CaCl₂ (820 mg, 7.4 mmol), NaOH (44 mg, 1.1 mmol), i-PrOH (10 mL) and H₂O (10 mL) providing 200 mg of the title compound as a solid. After purification, the two batches (350 mg+200 mg) were combined to provide a total of 550 mg of the title compound. HPLC retention time=5.24 min. Column: Ultimate XB-C18, 3 μm, 3.0*50 mm. Mobile Phase: 1.0% MeCN in water (0.1% TFA) to 5% MeCN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 min, and hold two minutes. Flow rate: 1.2 mL/min. LCMS: MS=528.3 (M+H).

### Preparation 11

### (S)-2-N9H-fluoren-9-yl)methoxy)carbonyl)amino)-3-(1-(tert-butoxycarbonyl)-6-ethyl-1H-indol-3-yl)propanoic Acid

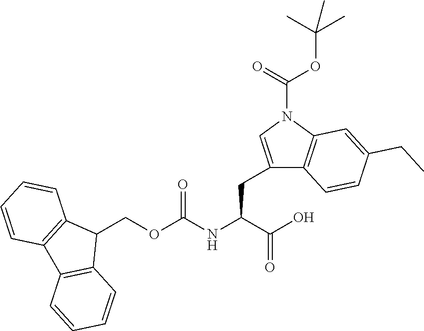 [SMILES: CCc1ccc2c(C[C@H](NC(=O)OCC3c4ccccc4-c4ccccc43)C(=O)O)cn(C(=O)OC(C)(C)C)c2c1]

### Step 1

### Preparation of tert-butyl-6-bromo-3-formyl-1H-indole-1-carboxylate

To a 250 mL round bottom flask was added a solution of 6-bromo-1H-indole-3-carbaldehyde (9.5 g, 42.4 mmol) in DCM (150 mL) followed by DMAP (6.7 g, 55.1 mmol). To the mixture was then added Boc₂O (13.9 g, 63.6 mmol) in portions over 10 mins and the resulting reaction mixture stirred at room temperature (25° C.) for 16 h. The reaction mixture was diluted with DCM (200 mL) and washed with an aqueous solution of 10% citric acid (200 mL×2), water (200 mL×2), brine (200 mL), dried over Na₂SO₄, filtered and concentrated under reduced pressure to provide 13.0 g of the title compound as a solid. LCMS: MS=223.6 (M−100)

### Step 2

### Preparation of tert-butyl 3-(2-(((benzyloxy)carbonyl)amino)-3-methoxy-3-oxoprop-1-en-1-yl)-6-bromo-1H-indole-1-carboxylate

To a 500 mL round bottom flask was added (±)-methyl 2-benzyloxycarbonylamino-2-(dimethoxyphosphinyl) acetate (14.6 g, 44.1 mmol), DCM (150 mL) and DBU (7.3 g, 48.1 mmol). The mixture was then cooled to 0° C. using an ice-water bath. In a second flask tert-butyl-6-bromo-3-formyl-1H-indole-1-carboxylate (13.0 g, 40.1 mmol) was dissolved in DCM (150 mL) and this solution was then added drop wise via syringe to the reaction mixture at 0° C. After the addition the mixture was stirred at 0° C. for 30 minutes. The ice-water bath was then removed and the mixture was allowed to warm to room temperature (15° C.) and the reaction stirred at this temperature for 16 h. The mixture was diluted with DCM (100 mL) and washed with aqueous 5% citric acid (200 mL), brine (200 mL), dried over anhydrous sodium sulfate, filtered and concentrated under reduced pressure. The resulting residue was purified via flash chromatography on a 120 g column employing a petroleum ether/EtOAc solvent mixture (gradient from 100/10% to 80/20%). The fractions containing the desired product were combined and concentrated under reduced pressure to provide 16 g of the title compound as a solid. LCMS: MS=551.1 (M+23)

### Step 3

### Preparation of tert-butyl (S)-3-(2-(((benzyloxy)carbonyl)amino)-3-methoxy-3-oxopropyl)-6-bromo-1H-indole-1-carboxylate

To a 250 mL parr bottle was added tert-butyl 3-(2-(((benzyloxy)carbonyl)amino)-3-methoxy-3-oxoprop-1-en-1-yl)-6-bromo-1H-indole-1-carboxylate (6.5 g, 12.3 mmol) and (+)-1,2-Bis((2S,5S)-2,5-diethylphospholano)benzene(1,5-cyclooctadiene)rhodium(1) tetrafluoroborate (250 mg, 0.38 mmol), followed by MeOH (100 mL—the MeOH was pre-treated by bubbling N₂ gas via a syringe through the solvent for 20 minutes). The resulting reaction mixture was degassed by vacuum and purged with N₂ gas 6 times. The container was then back filled with H₂ and then degassed, purged and refilled 3 times. The container was then pressurized with H₂ gas to 50 psi and was stirred at 50° C. for 4 days. The mixture was cooled to 28° C. then concentrated under reduced pressure. The resulting crude product was purified by reverse phase HPLC on a C-18 column employing a MeCN/H₂O solvent mixture (gradient from 100/0% to 0/100%). The fractions with the desired product were combined and lyophilized to provide 5.1 g of the title compound as a solid. ¹H NMR (400 MHz, CDCl₃) δ 8.31 (s, 1H), 7.39-7.26 (m, 8H), 5.37 (d, 8.0 Hz, 1H), 5.15-5.07 (m, 2H), 4.73-4.69 (m, 1H), 3.69 (s, 3H), 3.27-3.15 (m, 2H), 1.66 (s, 9H). LCMS: MS=554.4 (M+23)

### Step 4

### Preparation of tert-butyl (S)-3-(2-(((benzyloxy)carbonyl)amino)-3-methoxy-3-oxopropyl)-6-ethyl-1H-indole-1-carboxylate

In a 100 mL round bottom flask tert-butyl-(S)-3-(2-(((benzyloxy)carbonyl)amino)-3-methoxy-3-oxopropyl)-6-bromo-1H-indole-1-carboxylate (1.8 g, 3.4 mmol) was dissolved in anhydrous dioxane (40 mL). The reaction mixture was degassed under vacuum and back filled with N₂ gas 3 times. To the reaction was then added drop wise a 1 M solution of Et₂Zn in toluene (6.8 mL, 6.8 mmol) followed by Pd(dppf)Cl₂ (124 mg, 0.17 mmol). The resulting reaction mixture was degassed under vacuum and back filled with N₂ gas 3 times and then heated to 100° C. for 3 h. The reaction mixture was cooled to 25° C. and then quenched with saturated aqueous NH₄Cl solution (100 mL). The reaction mixture was extracted with EtOAc (3×80 mL) and the combined organic extracts were dried over anhydrous Na₂SO₄, filtered and concentrated under reduced pressure. The crude product was purified by flash chromatography on silica gel with a petroleum ether/EtOAc solvent mixture (gradient from 100/0% to 70/30%). The fractions containing the desired product were combined and concentrated to provide 1.5 g the title compound as an oil. LCMS: MS=503.2 (M+23)

### Step 5

### Preparation of tert-butyl (S)-3-(2-amino-3-methoxy-3-oxopropyl)-6-ethyl-1H-indole-1-carboxylate

To a 100 mL round bottom flask was added a solution of tert-butyl (S)-3-(2-(((benzyloxy)carbonyl)amino)-3-methoxy-3-oxopropyl)-6-ethyl-1H-indole-1-carboxylate (1.6 g, 3.3 mmol) in triethylamine (0.9 mL) and MeOH (30 mL). To the mixture was then added dry 10% wt Pd/C (709 mg, 0.7 mmol). The resulting suspension mixture was degassed under vacuum and back filled with N₂ gas 3 times. It was then degassed under vacuum and back filled with H₂ gas 3 times. The resulting mixture was degassed under vacuum and back filled with H₂ gas employing a balloon of H₂ gas. The mixture was stirred at 28° C. for 3 h then passed through Kieselguhr filter and the filtrate concentrated under reduced pressure to provide 1.1 g of the crude title compound as an oil. LCMS: MS=346.8

### Step 6

### Preparation of tert-butyl (S)-3-(2-((((9H-fluoren-9-yl)methoxy)carbonyl)amino)-3-methoxy-3-oxopropyl)-6-ethyl-1H-indole-1-carboxylate

To a 100 mL round bottom flask containing crude tert-butyl (S)-3-(2-amino-3-methoxy-3-oxopropyl)-6-ethyl-1H-indole-1-carboxylate (1.1 g, 3.2 mmol) was added dioxane (30 mL) and water (10 mL). To the resulting solution was added Na₂CO₃ (1.7 g, 15.9 mmol) and the mixture then cooled to 0° C. using an ice-water bath. To the reaction was added Fmoc-OSu (2.7 g, 7.9 mmol) in portions over 5 minutes and then the ice-water bath was removed. The reaction was then stirred at room temperature (28 C) for about 6 h. The reaction mixture was diluted with water (100 mL) and extracted with EtOAc (2×100 mL). The combined organic extracts were dried over sodium sulfate, filtered and concentrated under reduced pressure. Purification of the crude material by flash chromatography on an 80 g silica gel column with a petroleum ether/EtOAc solvent mixture (gradient from 100/0% to 90/0%) provided 2.0 g of a crude oil. The oil was then purified by reverse phase HPLC on a C-18 column (120 g) employing a MeCN/H₂O solvent mixture (gradient from 100/0% to 80/20%). The fractions with the desired product were combined and concentrated to provide 1.0 g of the title compound as an oil. LCMS: MS=591.3 (M+23)

### Step 7

### Preparation of (S)-2-((((9H-fluoren-9-yl)methoxy)carbonyl)amino)-3-(1-(tert-butoxycarbonyl)-6-ethyl-1H-indol-3-yl)propanoic Acid

To a 100 mL round bottom flask was added i-PrOH (35 mL), H₂O (15 mL) and anhydrous CaCl₂ (4.4 g, 40.0 mmol). The mixture was stirred at room temperature (28° C.) for 10 minutes. In a second 100 mL round bottom flask was added tert-butyl (S)-3-(2-((((9H-fluoren-9-yl)methoxy)carbonyl)amino)-3-methoxy-3-oxopropyl)-6-ethyl-1H-indole-1-carboxylate (900 mg, 1.6 mmol), the reaction mixture from the first round bottom flask, followed by NaOH (89 mg, 2.2 mmol). The resulting reaction mixture was stirred at room temperature (28° C.) for 16 h. The reaction mixture was diluted with H₂O (˜100 mL) and was extracted with EtOAc (2×˜100 mL). The combined organic extracts were dried over anhydrous Na₂SO₄, filtered and concentrated under reduced pressure to provide a crude residue. The above reaction was then repeated using the following quantities of reagents: tert-butyl (S)-3-(2-((((9H-fluoren-9-yl)methoxy)carbonyl)amino)-3-methoxy-3-oxopropyl)-6-ethyl-1H-indole-1-carboxylate (100 mg, 0.18 mmol), CaCl₂ (888 mg, 8.0 mmol), NaOH (9.9 mg, 0.25 mmol), i-PrOH (7 mL) and H₂O (3 mL). Purification of the combined crude residues by flash chromatography on a 40 g silica gel column with a DCM/MeOH solvent mixture (gradient from 100/0% to 90/0%) provided 850 mg of the title compound as an oil. The oil was purified by preparatory SFC to provide 670 mg of the title compound as an oil. The oil was purified further by reverse phase HPLC on a C-18 column (120 g), eluting with a MeCN/H₂O solvent mixture (gradient from 100/0% to 0/100%). The fractions containing the desired product were combined and concentrated by lyophilization to provide 350 mg of the title compound as a solid. LCMS: MS=577.2 (M+23). HPLC retention time=5.51 min. Column: Ultimate XB-C18, 3 μm, 3.0*50 mm. Mobile Phase: 1.0% MeCN in water (0.1% TFA) to 5% MeCN in water (0.1% TFA) in 1 min; then from 5% MeCN in water (0.1% TFA) to 100% MeCN (0.1% TFA) in 5 minutes; hold at 100% MeCN (0.1% TFA) for 2 minutes; back to 1.0% MeCN in water (0.1% TFA) at 8.01 min, and hold two minutes. Flow rate: 1.2 mL/min.

In the assays below, the following the abbreviations and definitions may be referred to:

APC is allophycocyanin;

BSA is bovine serum albumin;

DMSO is dimethyl sulphoxide;

EDTA is ethylenediaminetetraacetic acid;

FBS is fetal bovine serum;

HBBS is Hanks' Balanced Salt Solution;

HTS is high throughput screen;

HWB is human whole blood;

MF is mean fluorescence;

PBS is phosphate-buffered saline;

K₂EDTA is ethylenediaminetetraacetic acid dipotassium salt;

TNF-α is tumor necrosis factor-alpha;

C5a is complement component 5a; and

HEPES is 4-(2-hydroxyethyl)-1-piperazineethanesulfonic acid.

### Biological Assays

### HWB—Oxidative Burst Assay

Inhibition of oxidative burst activity was determined in HWB primed with TNF-α and stimulated with C5a. C5a-induced oxidative burst response (generation of reactive oxygen species) was then detected in the presence of luminol as a chemiluminescent signal.

On the day of assay, HWB was collected from a healthy, non-medicated volunteer into tubes containing 3.8% sodium citrate (final sodium citrate concentration in HWB is 0.38%) and stored in a 37° C. water bath until use (no longer than 60 minutes).

To start the assay, TNF-α was added to HWB to a final concentration of 1.0 nM and 68 μL of this HWB/TNF-α mix was transferred to each well of a 384-well white Optiplate (assay plate). 4.0 μL of various concentrations of test agent were then added to the assay plate and mixed twice gently by aspirating up and down. The assay plate was then placed on a thermoshaker (JITTERBUG-4) and incubated at 37° C. (without shaking). After 60 minutes, C5a (Complement Technology) prepared in luminol (Sigma) was added to the assay plate and mixed twice gently by aspirating up and down. The plate was then placed in a ViewLux 1430 Microplate Imager (Perkin Elmer). After 5 minutes, oxidative burst activity was determined with the ViewLux by measuring luminol-enhanced whole blood chemiluminescence. Final assay conditions were 1.5 mM HEPES pH 7.5, 0.015% BSA, 15% HBSS, 0.85 ng/mL TNFα, 1.0 mM Luminol, C5a 20 nM, 76.5% HWB, 0.15% DMSO and various concentrations of test agent.

The percent (%) effect at each concentration of test agent was then calculated based on and relative to the amount of signal that was produced by positive (i.e. full inhibition of C5a induced oxidative burst) and negative (i.e. completely uninhibited C5a induced oxidative burst) control wells contained within each assay plate. The concentration and % effect values for test agents were plotted and the concentration of test agent required for 50% effect (IC₅₀) was determined with a four-parameter logistic dose response equation (BioBook; IDBS). Kb (nM) was then calculated (BioBook; IDBS) using the equation described by Leff and Dougal (TIPS 1993 14:110-112).

### HWB—CD11b Expression Assay

HWB was collected from healthy, non-medicated volunteers into BD Vacutainer® blood collection tubes with K₂EDTA as an anticoagulant. HWB was aliquoted (85 μL/well) in 96-well, deep-well, V-bottom plates and incubated at 37° C. for 30 minutes. Test compounds (5 μL/well) were added to HWB and incubated at 37° C. for 30 minutes. Anti-human CD11b antibody conjugated with APC (BD Biosciences) was added to HWB (5 μL/well) and incubated at 37° C. for additional 30 minutes. HWB was then challenged with human C5a (final 1 nM) for 30 minutes at 37° C. Samples were treated with warm 1× Lyse/Fix buffer (BD Biosciences) to terminate activation and further incubated at 37° C. for 20 minutes to lyse red blood cells. Assay plates were centrifuged at 300×g for 5 minutes, supernatant was aspirated, and cells were washed once with PBS containing 0.5% FBS. Washed cells were resuspended in PBS containing 0.5% FBS and subjected to flow cytometric analysis using an LSRFortessa equipped with a HTS plate loader (BD Biosciences). The granulocyte population was gated for analysis of MF of CD11b staining using FACSDiva version 6.2 (BD Biosciences). Data from 11 compound concentrations (singlicate at each concentration) was normalized as a percentage of control based on the formula:

% of Control=100×(A−B)/(C−B)

where A is MF from wells containing test compound and C5a, B is MF from wells without C5a (background MF from unstimulated samples) and C is MF from wells containing only C5a (maximum MF). Inhibition curves and IC₅₀ values (test compound concentrations which produce 50% inhibition) were determined using the Prism version 5 software (GraphPad). HWB was also stimulated with 11 different concentrations of C5a to construct a responsive curve and to obtain EC₅₀ value (the concentration of C5a that gives half-maximal response) and curve slope using the Prism version 5 software. Kb (nM) was then calculated using the equation described by Leff and Dougal (TIPS 1993 14:110-112).

TABLE 1
Examples 1 to 15

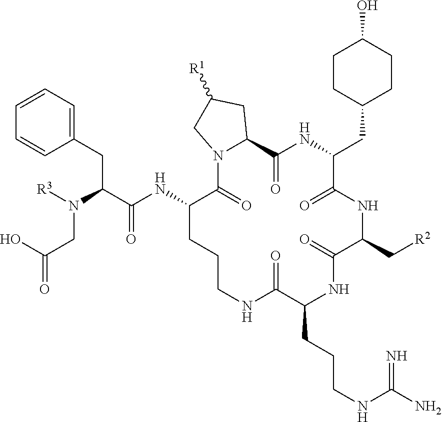

| Ex | 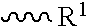 | R² | R³ | Oxidative n burst, Kb | | CD11b, n Kb | |
|---|---|---|---|---|---|---|---|
| 1 | 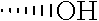 | 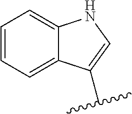 [SMILES: *c1c[nH]c2ccccc12] | H | 1.6 | 7 | 13.1 | 14 |
| 2 | 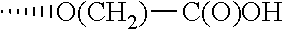 | 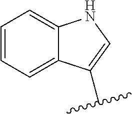 [SMILES: *c1c[nH]c2ccccc12] | H | 3.0 | 7 | 28.8 | 28 |
| 3 | 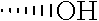 | 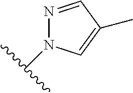 [SMILES: *n1cc(C)cn1] | H | 21.6 | 3 | 212.1 | 2 |
| 4 | 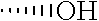 | 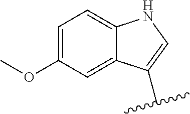 [SMILES: *c1c[nH]c2ccc(OC)cc12] | H | 3.0 | 2 | 53.8 | 4 |
| 5 | H | 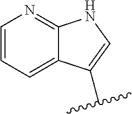 [SMILES: *c1c[nH]c2ncccc12] | H | 27.3 | 3 | 300.0 | 4 |
| 6 | 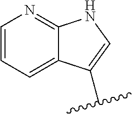 [SMILES: *c1c[nH]c2ncccc12] | 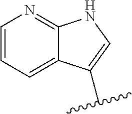 [SMILES: *c1c[nH]c2ncccc12] | H | 68.1 | 3 | 459.8 | 2 |
| 7 | 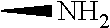 | 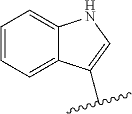 [SMILES: *c1c[nH]c2ccccc12] | H | 1.8 | 5 | 10.9 | 10 |
| 8 | 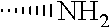 | 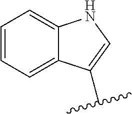 [SMILES: *c1c[nH]c2ccccc12] | H | 4.0 | 6 | 19.8 | 10 |
| 9 | 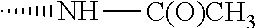 | 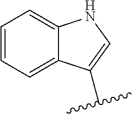 [SMILES: *c1c[nH]c2ccccc12] | H | 1.0 | 4 | 32.6 | 6 |
| 10 | 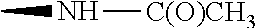 | 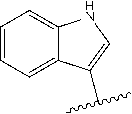 [SMILES: *c1c[nH]c2ccccc12] | H | 2.1 | 6 | 11.7 | 4 |
| 11 | 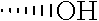 | 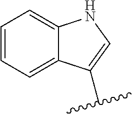 [SMILES: *c1c[nH]c2ccccc12] | CH₃ | 1.2 | 4 | NT | |
| 12 | 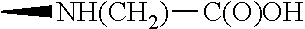 | 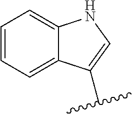 [SMILES: *c1c[nH]c2ccccc12] | H | 1.3 | 4 | 32.6 | 2 |
| 13^{D} | 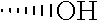 | 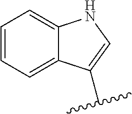 [SMILES: *c1c[nH]c2ccccc12] | H | 1.3 | 3 | NT | |
| 14 | 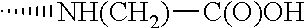 | 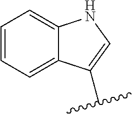 [SMILES: *c1c[nH]c2ccccc12] | H | 1.7 | 3 | NT | |
| 15 | 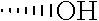 | 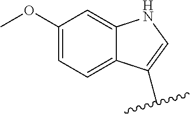 [SMILES: *c1c[nH]c2cc(OC)ccc12] | H | 2.2 | 1 | NT | |
| 16 | 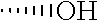 | 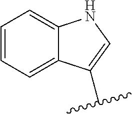 [SMILES: *c1c[nH]c2ccccc12] | H | 6.5 | 3 | 75.2 | 3 |
| 17 | 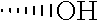 | 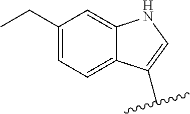 [SMILES: *c1c[nH]c2cc(CC)ccc12] | H | 29.7 | 3 | 233.9 | 3 |

^{D}Example 13 is the “3,5-dideuterophenyl” derivative of Example 1
NT = not tested
n = number of assays performed

1. A compound of formula (Ia) or formula (Ib) 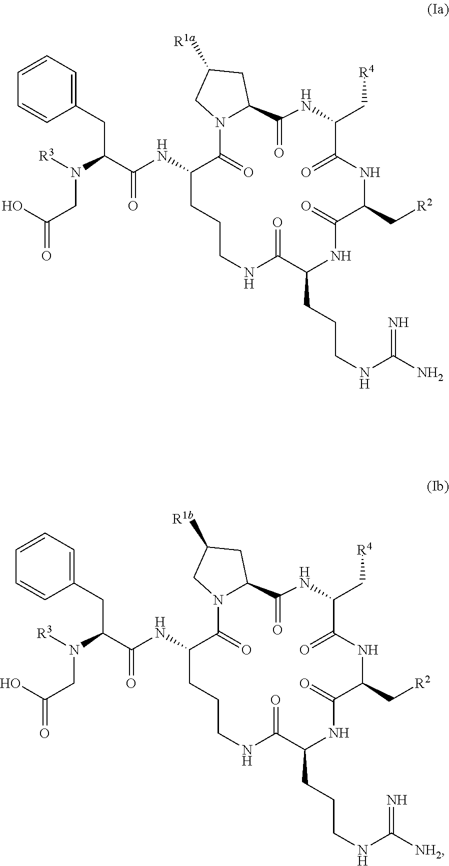
or a pharmaceutically acceptable salt thereof, wherein:
R^{1a} is H, OH, O(CH₂)—C(O)OR⁶, NH₂, NH—C(O)R⁵ or NH(CH₂)—C(O)OR⁶;
R^{1b} is NH₂, NH—C(O)R⁵ or NH(CH₂)—C(O)OR⁶;
R² is a 5-, 6-, 9- or 10-membered heteroaryl containing one, two or three nitrogen atoms and wherein the heteroaryl is optionally substituted on a ring carbon atom with one or two R⁷;
R³ is hydrogen or C₁-C₄ alkyl;
R⁴ is 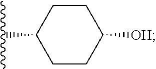 [SMILES: *[C@H]1CC[C@@H](O)CC1]
R⁵ is C₁-C₄ alkyl;
R⁶ is H or C₁-C₄ alkyl; and
R⁷ is C₁-C₄ alkyl or C₁-C₄ alkoxy.

2. A compound according to claim 1, or a pharmaceutically acceptable salt thereof, wherein R^{1a} is OH, O(CH₂)—C(O)OR⁶, NH₂, NH—C(O)R⁵ or NH(CH₂)—C(O)OR⁶; or R^{1b} is NH₂, NH—C(O)R⁵ or NH(CH₂)—C(O)OR⁶.

3. A compound according to claim 1 of formula (Ia), or a pharmaceutically acceptable salt thereof, wherein R^{1a} is OH or O(CH₂)—C(O)OR⁶.

4. A compound according to claim 1 of formula (Ia), or a pharmaceutically acceptable salt thereof, wherein R^{1a} is OH.

5. A compound according to claim 1 of formula (Ib), or a pharmaceutically acceptable salt thereof, wherein R^{1b} is NH₂, NH—C(O)R⁵ or NH(CH₂)—C(O)OR⁶.

6. A compound according to claim 1 or a pharmaceutically acceptable salt thereof, wherein R² is a 9-membered heteroaryl containing one or two nitrogen atoms and wherein the heteroaryl is optionally substituted on a ring carbon atom with one or two R⁷.

7. A compound according to claim 1, or a pharmaceutically acceptable salt thereof, wherein R² is a heteroaryl selected from 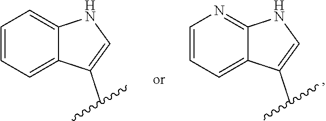 [SMILES: *c1c[nH]c2ccccc12 ; *c1c[nH]c2ncccc12]
wherein said heteroaryl is optionally substituted on a ring carbon atom with R⁷.

8. A compound according to claim 1, or a pharmaceutically acceptable salt thereof, wherein R² is the heteroaryl 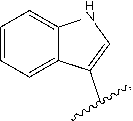 [SMILES: *c1c[nH]c2ccccc12]
and said heteroaryl is optionally substituted on a ring carbon atom with R⁷.

9. A compound according to claim 1, or a pharmaceutically acceptable salt thereof, wherein R⁷ is methyl or methoxy.

10. A compound according to claim 1, or a pharmaceutically acceptable salt thereof, wherein R³ is H.

11. A compound according to claim 1, or a pharmaceutically acceptable salt thereof, selected from:
N-[(2S)-1-{[(3R,6S,9S,15S,19R,20aS)-9-(3-carbamimidamidopropyl)-19-hydroxy-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine;
N-[(2S)-1-{[(3R,6S,9S,15S,19R,20aS)-9-(3-carbamimidamidopropyl)-19-(carboxymethoxy)-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine;
N-[(2S)-1-({(3R,6S,9S,15S,19R,20aS)-9-(3-carbamimidamidopropyl)-19-hydroxy-3-[(cis-4-hydroxycyclohexyl)methyl]-6-[(4-methyl-1H-pyrazol-1-yl)methyl]-1,4,7,10,16-pentaoxoicosahydropyrrolo[1,2-a][1,4,7,10,13]pentaazacyclooctadecin-15-yl}amino)-1-oxo-3-phenylpropan-2-yl]glycine;
N-[(2S)-1-({(3R,6S,9S,15S,19R,20aS)-9-(3-carbamimidamidopropyl)-19-hydroxy-3-[(cis-4-hydroxycyclohexyl)methyl]-6-[(5-methoxy-1H-indol-3-yl)methyl]-1,4,7,10,16-pentaoxoicosahydropyrrolo[1,2-a][1,4,7,10,13]pentaazacyclooctadecin-15-yl}amino)-1-oxo-3-phenylpropan-2-yl]glycine;
N-[(2S)-1-{[(3R,6S,9S,15S,20aS)-9-(3-carbamimidamidopropyl)-3-[(cis-4-hydroxycyclohexyl)methyl]-1,4,7,10,16-pentaoxo-6-(1H-pyrrolo[2,3-b]pyridin-3-ylmethyl)icosahydropyrrolo[1,2-a][1,4,7,10,13]pentaazacyclooctadecin-15-yl]amino}-1-oxo-3-phenylpropan-2-yl]glycine;
N-[(2S)-1-{[(3R,6S,9S,15S,19R,20aS)-9-(3-carbamimidamidopropyl)-19-hydroxy-3-[(cis-4-hydroxycyclohexyl)methyl]-1,4,7,10,16-pentaoxo-6-(1H-pyrrolo[2,3-b]pyridin-3-ylmethyl)icosahydropyrrolo[1,2-a][1,4,7,10,13]pentaazacyclooctadecin-15-yl]amino}-1-oxo-3-phenylpropan-2-yl]glycine;
N-[(2S)-1-{[(3R,6S,9S,15S,19S,20aS)-19-amino-9-(3-carbamimidamidopropyl)-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine;
N-[(2S)-1-{[(3R,6S,9S,15S,19R,20aS)-19-amino-9-(3-carbamimidamidopropyl)-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine;
N-[(2S)-1-{[(3R,6S,9S,15S,19R,20aS)-19-(acetylamino)-9-(3-carbamimidamidopropyl)-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine;
N-[(2S)-1-{[(3R,6S,9S,15S,19S,20aS)-19-(acetylamino)-9-(3-carbamimidamidopropyl)-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine;
N-[(2S)-1-{[(3R,6S,9S,15S,19R,20aS)-9-(3-carbamimidamidopropyl)-19-hydroxy-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]-N-methylglycine;
{[(2S)-1-{[(3R,6S,9S,15S,19S,20aS)-9-(3-carbamimidamidopropyl)-19-[(carboxymethyl)amino]-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]amino}acetic acid;
N-{(2S)-1-{[(3R,6S,9S,15S,19R,20aS)-9-(3-carbamimidamidopropyl)-19-hydroxy-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-(3,5-dideuterophenyl)propan-2-yl}glycine;
{[(2S)-1-{[(3R,6S,9S,15S,19R,20aS)-9-(3-carbamimidamidopropyl)-19-[(carboxymethyl)amino]-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]amino}acetic acid;
N-[(2S)-1-({(3R,6S,9S,15S,19R,20aS)-9-(3-carbamimidamidopropyl)-19-hydroxy-3-[(cis-4-hydroxycyclohexyl)methyl]-6-[(6-methoxy-1H-indol-3-yl)methyl]-1,4,7,10,16-pentaoxoicosahydropyrrolo[1,2-a][1,4,7,10,13]pentaazacyclooctadecin-15-yl}amino)-1-oxo-3-phenylpropan-2-yl]glycine;
((S)-1-(((3R,6S,9S,15S,19R,20aS)-6-((1H-indazol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine; and
((S)-1-(((3R,6S,9S,15S,19R,20aS)-6-((6-ethyl-1H-indol-3-yl)methyl)-9-(3-guanidinopropyl)-19-hydroxy-3-(((1s,4S)-4-hydroxycyclohexyl)methyl)-1,4,7,10,16-pentaoxoicosahydropyrrolo[1,2-a][1,4,7,10,13]pentaazacyclooctadecin-15-yl)amino)-1-oxo-3-phenylpropan-2-yl)glycine.

12. N [(2S)-1-{[(3R,6S,9S,15S,19R,20aS)-9-(3-carbamimidamidopropyl)-19-hydroxy-3-[(cis-4-hydroxycyclohexyl)methyl]-6-(1H-indol-3-ylmethyl)-1,4,7,10,16-pentaoxoicosahydropyrrolo[1,2-a][1,4,7,10,13]pentaazacyclooctadecin-15-yl]amino}-1-oxo-3-phenylpropan-2-yl]glycine or a pharmaceutically acceptable salt thereof.

13. A pharmaceutical composition comprising a compound according to claim 1, or a pharmaceutically acceptable salt thereof, and a pharmaceutically acceptable excipient.

14. The pharmaceutical composition according to claim 13 further comprising one or more additional therapeutic agents.

15-18. (canceled)

19. A method of treating a disorder in a human or animal for which a C5a antagonist is indicated, comprising administering to said human or animal in need of such treatment a therapeutically effective amount of a compound according to claim 1, or a pharmaceutically acceptable salt thereof.

20. A compound according to claim 1, or a pharmaceutically acceptable salt thereof, in combination with another pharmacologically active compound, or with two or more other pharmacologically active compounds.

21. The method of claim 19 wherein the disorder is acute kidney injury.

22. A compound of structure 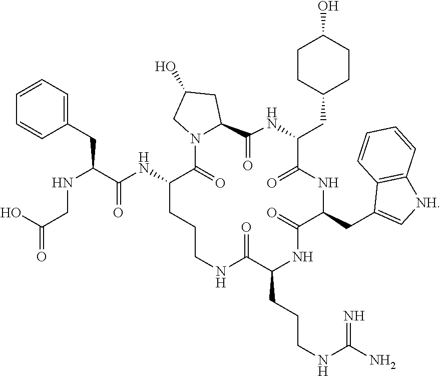 [SMILES: N=C(N)NCCC[C@@H]1NC(=O)[C@H](Cc2c[nH]c3ccccc23)NC(=O)[C@@H](C[C@H]2CC[C@@H](O)CC2)NC(=O)[C@@H]2C[C@@H](O)CN2C(=O)[C@@H](NC(=O)[C@H](Cc2ccccc2)NCC(=O)O)CCCNC1=O]
